(19) United States
(12) Patent Application Publication
(10) Pub. No.: US 2024/0165421 A1
(43) Pub. Date: May 23, 2024

(54) STIMULATED CORTICAL RESPONSE

(71) Applicant: THE RESEARCH FOUNDATION FOR THE STATE UNIVERSITY OF NEW YORK, Albany, NY (US)

(72) Inventors: Stephen L. MACKNIK, Brooklyn, NY (US); Susana MARTINEZ-CONDE, Brooklyn, NY (US); Edward WHITE, Albany, NY (US); Satyavolu S. PAPA RAO, Albany, NY (US); Spyridon GALIS, Albany, NY (US); John N. CARTER, Brooklyn, NY (US); Olivya CABALLERO, Brooklyn, NY (US)

(73) Assignee: THE RESEARCH FOUNDATION FOR THE STATE UNIVERSITY OF NEW YORK, Albany, NY (US)

(21) Appl. No.: 18/546,166

(22) PCT Filed: Feb. 12, 2022
(86) PCT No.: PCT/US2022/016242

§ 371 Date: Aug. 11, 2023

Related U.S. Application Data
(60) Provisional application No. 63/149,130, filed on Feb. 12, 2021.

(51) Int. Cl. A61N 5/06

(52) U.S. Cl. CPC A61N 5/0601; A61N 5/0622; A61N 2005/0659; A61N 2005/0663; A61N 5/067

(57) ABSTRACT

There is set forth herein: an implant adapted for implantation in a user having a neocortex at least part of which has been made responsive to light, the neocortex including a plurality of columns forming an array of cortical columns capable of description by a cortical map characterizing, identifying or defining a location or topographical relationship and placement for respective ones of the plurality of columns; wherein the implant includes an emitter array; wherein the emitter array includes a plurality of emitters, wherein respective ones of the plurality of emitters are configured to emit light toward the array of cortical columns.

### CROSS-REFERENCE TO RELATED APPLICATIONS

This application is a national stage filing under section 371 of International Application No. PCT/US2022/016242 filed on Feb. 12, 2022, titled “Stimulated Cortical Response” and published on Aug. 18, 2022, as WO2022/174123A1 and claims the benefit of priority of U.S. Provisional Application No. 63/149,130 filed Feb. 12, 2021, titled “Methods, Compositions, and Devices for the Restoration of Visual Responses”, which Provisional Application No. 63/149,130 is incorporated by reference herein in its entirety. WO2022/174123A1 is incorporated by reference herein in its entirety.

### BACKGROUND

Embodiments herein relate generally to responses, and specifically to cortical response.

The brain of various organisms comprises a cerebrum that includes cerebral cortex. The cerebral cortex can be involved in processing sensory information, motor function, planning and organization, and language processing. The cerebral cortex can include sensory areas such as the visual cortex, the auditory cortex, and other sensory areas. Additional areas of a brain can include the cerebellum and brain stem.

### BRIEF DESCRIPTION

There is set forth herein, in one aspect, a system. The system can include an implant adapted for implantation in a user having a neocortex at least part of which has been made responsive to light, the neocortex including a plurality of columns forming an array of cortical columns capable of description by a cortical map characterizing, identifying or defining a location or topographical relationship and placement for respective ones of the plurality of columns; wherein the implant includes an emitter array; wherein the emitter array includes a plurality of emitters, wherein respective ones of the plurality of emitters are configured to emit light toward the array of cortical columns capable of description by the cortical map characterizing, identifying or defining a location or topographical relationship and placement for respective ones of the plurality of columns.

There is set forth herein, in one aspect, a system. The system can include an implant adapted for implantation in a user having a neocortex at least part of which has been made responsive to light, the neocortex defined by a cortical map characterized by a plurality of columns; a plurality of emitters, wherein respective ones of the plurality of emitters are configured to emit light toward the cortical map characterized by the plurality of columns of the neocortex of the user; a plurality of detectors, wherein respective ones of the plurality of detectors are configured to detect response signals from brain tissue of the user that has been excited by a light emission of one or more emitter of the plurality of emitters.

There is set forth herein, in one aspect, a system. The system can include a plurality of emitters.

There is set forth herein, in one aspect, a system. The system can include a plurality of detectors

### DRAWINGS

These and other features, aspects, and advantages set forth herein will become better understood when the following detailed description is read with reference to the accompanying drawings in which like characters represent like parts throughout the drawings, wherein:

FIG. 1A is a block diagram illustrating a system having an implant system, a local system, an eyewear system and a remote system according to one embodiment;

FIG. 1B depicts a vision system including a retina, a lateral geniculate nucleus, and primary visual cortex (V1) according to one embodiment;

FIG. 1C depicts a schematic physiological representation of a cortical map according to one embodiment;

FIG. 1D depicts a schematic functional representation of cortical map according to one embodiment;

FIG. 1E depicts an emitter array and a detector array of an implant according to one embodiment;

FIG. 1F is a flowchart depicting a method for performance by an implant system interoperating with a local system, an eyewear system, and a remote system according to one embodiment;

FIG. 1G is schematic representation of an emitter array emitting light toward a cortical map according to one embodiment;

FIG. 1H is a block diagram of a computer system according to one embodiment;

FIGS. 2A-2D depict ON/OFF LGN input hypercolumns in V1;

FIG. 2E-H depicts long-term 2P fluorescence imaging from non-human primates (NHPs);

FIGS. 3A-3C depicts all-optical interrogation of a V1 neuronal population in awake NHP V1;

FIGS. 4A-4E depict data from NHP V1 convection-enhanced delivery (CED) of viruses, 2P Ca Imaging, and multi-color Aeq-FPs.

FIGS. 5A-5E depict a novel macaque NHP chamber in accordance with the present disclosure; FIGS. 6A-6D depict a conceptual approach to optogenetic inverse modeling of cortical functional architecture in the blind in accordance with the present disclosure;

FIGS. 7A-7E depict optogenetic stimulation in accordance with embodiments of the present disclosure;

FIGS. 8A-8F depict a spatiochromatic read-out and decode schema of the present disclosure;

FIGS. 9A-9H depict V1 hypercolumn LGN-Input (V1, Layer 4) as the primary organizing principle of the early visual system;

FIGS. 10A-10E depict 1P Optogenetic activation of LGN boutons in Layer 4, with Ca Imaging readout from V1 pyramidal neurons;

FIGS. 11A-11D depict an illustration of the PC spectrometer and detectors. FIG. 11B depicts an illustration of the photonic band in a 1D PC structure;

FIG. 12A depicts a bioluminescent Tet-Bow analysis;

FIG. 12B depicts a bioluminescent color separation in HSV color space;

FIG. 12C depicts A) high precision optical fiber coupling in accordance with the present disclosure;

FIG. 13A depict chip layouts of the present disclosure;

FIG. 13B depicts testing of the present disclosure;

FIG. 13C depicts a causal model of the transform between LGN Inputs and V1 Orientation preference of the present disclosure;

FIG. 14A depict a design of behavioral calibration experiments of the present disclosure.

FIG. 15A depicts a schematic view of a system including an emitter assembly defining an emitter array and a detector assembly defining detector array, according to embodiments of the disclosure;

FIG. 15B depicts a schematic view of a switch matrix of the emitter assembly of FIG. 15A, according to embodiments of the disclosure;

FIG. 15C depicts a schematic view of a portion of the emitter assembly of FIG. 15A including emitter devices, according to embodiments of the disclosure;

FIG. 15D depicts a side cross-sectional schematic view of the system of FIG. 15A, according to embodiments of the disclosure. M

### DETAILED DESCRIPTION

The accompanying figures, in which like reference numerals refer to identical or functionally similar elements throughout the separate views and which are incorporated in and form a part of the specification, further illustrate the present implementation(s) and, together with the detailed description of the implementation(s), serve to explain the principles of the present implementation(s). As understood by one of skill in the art, the accompanying figures are provided for ease of understanding and illustrate aspects of certain examples of the present implementation(s). The implementation(s) is/are not limited to the examples depicted in the figures.

The terms “connect,” “connected,” “contact” “coupled” and/or the like are broadly defined herein to encompass a variety of divergent arrangements and assembly techniques. These arrangements and techniques include, but are not limited to (1) the direct joining of one component and another component with no intervening components therebetween (i.e., the components are in direct physical contact); and (2) the joining of one component and another component with one or more components therebetween, provided that the one component being “connected to” or “contacting” or “coupled to” the other component is somehow in operative communication (e.g., electrically, fluidly, physically, optically, etc.) with the other component (notwithstanding the presence of one or more additional components therebetween). It is to be understood that some components that are in direct physical contact with one another may or may not be in electrical contact and/or fluid contact with one another. Moreover, two components that are electrically connected, electrically coupled, optically connected, optically coupled, fluidly connected or fluidly coupled may or may not be in direct physical contact, and one or more other components may be positioned therebetween.

The terms “including” and “comprising”, as used herein, mean the same thing.

The terms “substantially”, “approximately”, “about”, “relatively”, or other such similar terms that may be used throughout this disclosure, including the claims, are used to describe and account for small fluctuations, such as due to variations in processing, from a reference or parameter. Such small fluctuations include a zero fluctuation from the reference or parameter as well. For example, they can refer to less than or equal to ±10%, such as less than or equal to ±5%, such as less than or equal to ±2%, such as less than or equal to ±1%, such as less than or equal to ±0.5%, such as less than or equal to ±0.2%, such as less than or equal to ±0.1%, such as less than or equal to ±0.05%. If used herein, the terms “substantially”, “approximately”, “about”, “relatively,” or other such similar terms may also refer to no fluctuations, that is, +0%.

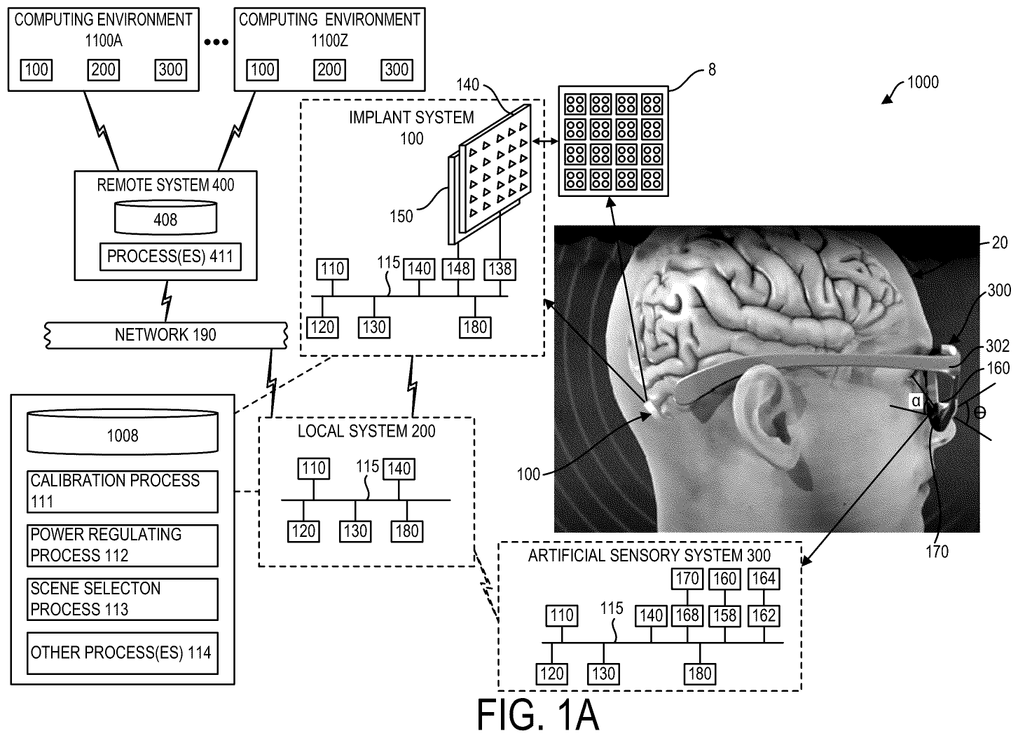

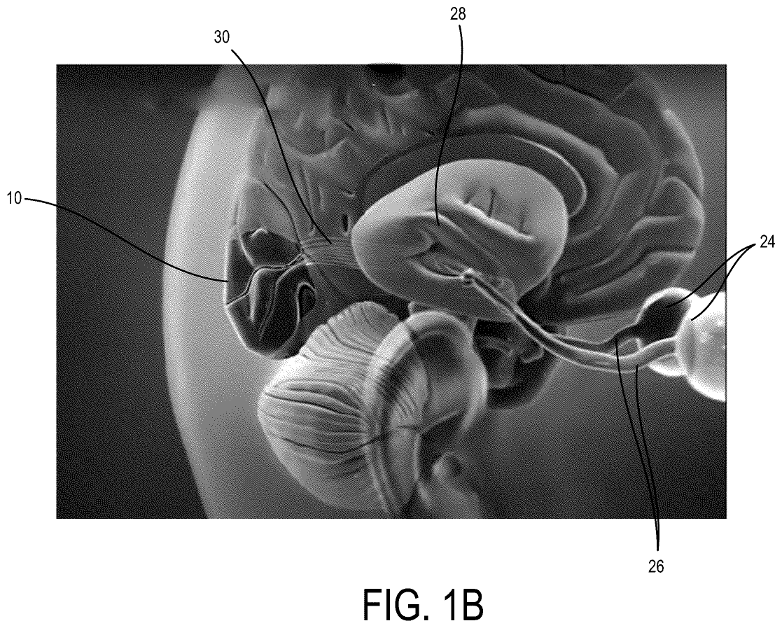

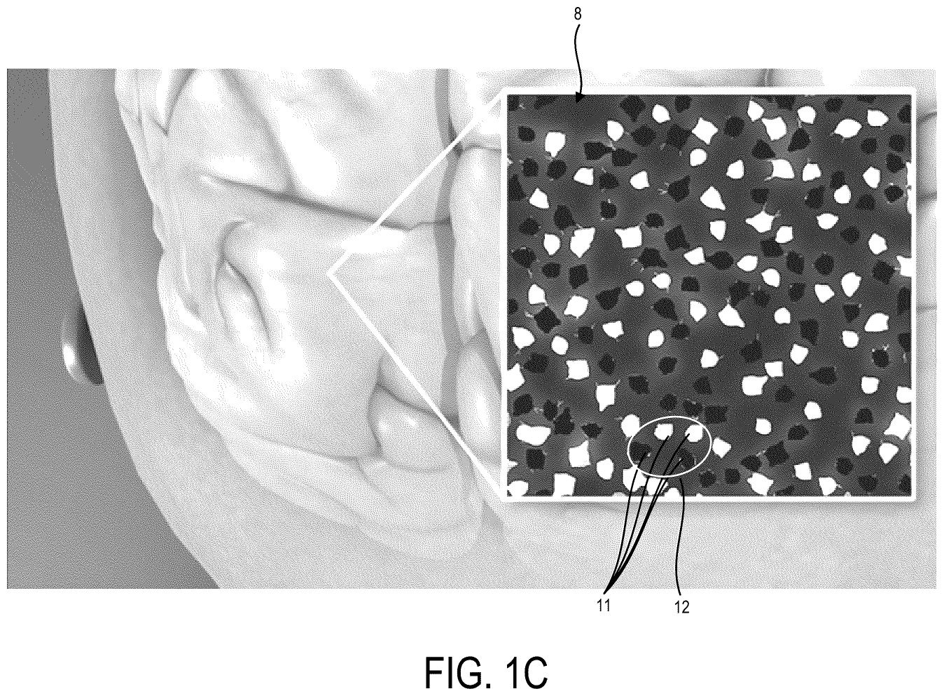

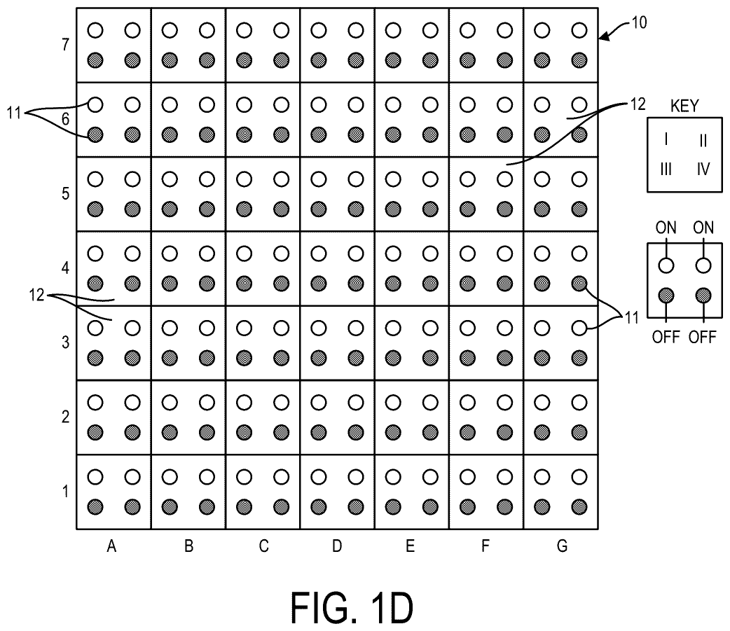

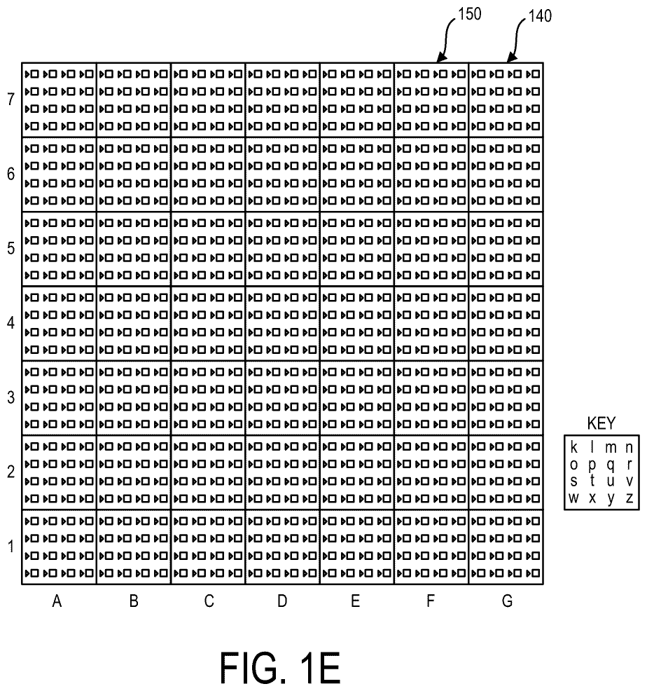

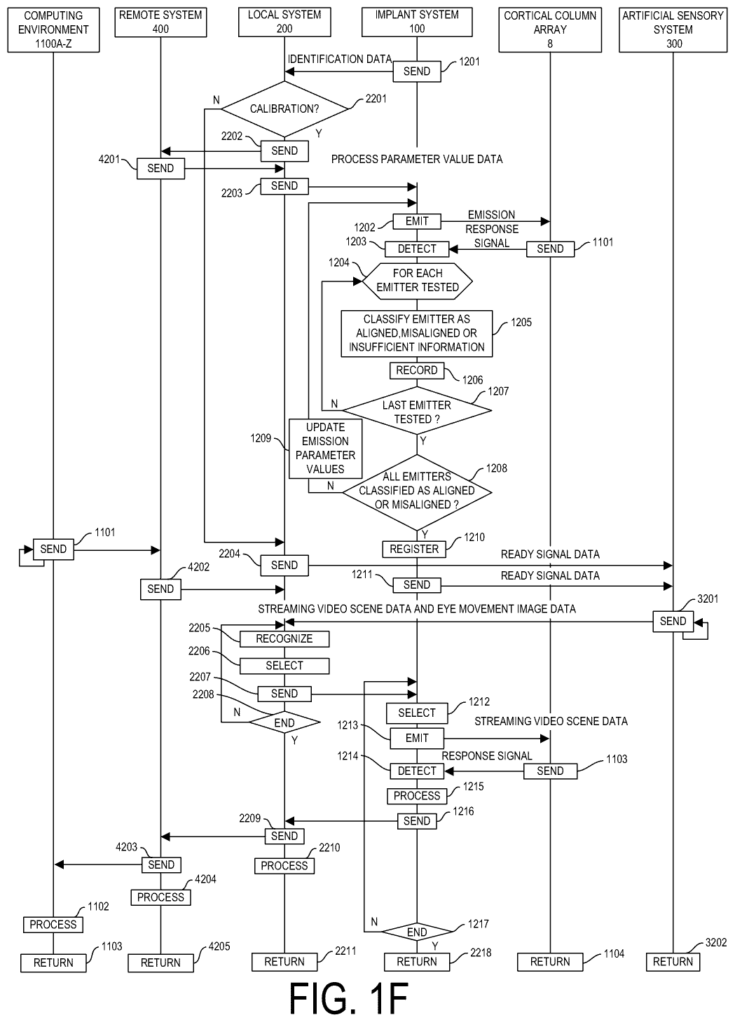

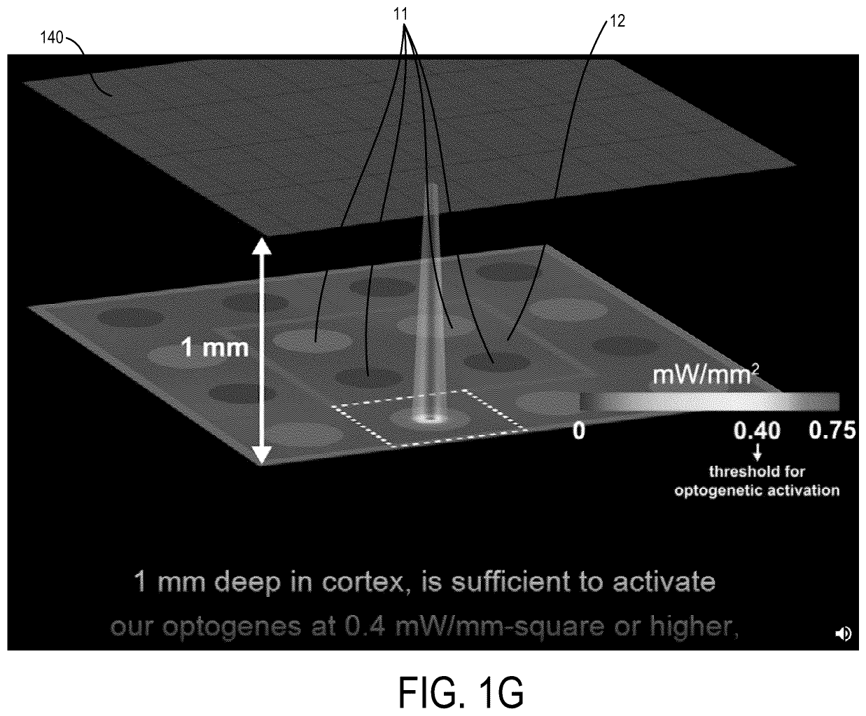

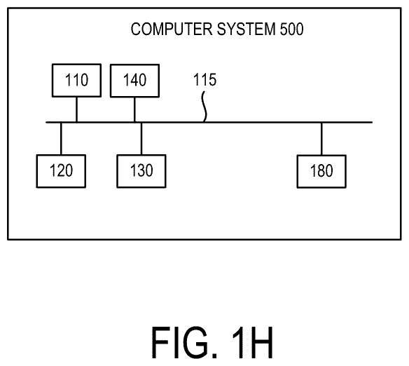

System 1000 for use in in stimulating thalamic inputs to neocortex is shown in FIG. 1A. The goal of such stimulation of thalamic inputs to the neocortex can be for restoration of lost sensory function, provision of synthetic sensory function, or recording of organic sensory function. In one embodiment, the thalamic inputs are lateral geniculate nucleus (LGN) projections to primary visual cortex (V1) of neocortex. In other embodiments, the thalamic input can originate from the medial geniculate nucleus, the ventral posterior medial (VPM) nucleus, the posterior medial (POm) thalamus, or other body of the thalamus. In other embodiments, the part of neocortex interacted with by System 1000 can be the primary auditory cortex (A1), primary somatosensory cortex (S1) or another cortical region of neocortex. System 1000 can include implant system 100, local system 200, artificial sensory system 300, and remote system 400. Remote system 400 can be in communication with each of implant system 100, local system 200, and artificial sensory system 300 via network 190. Network 190 can be a physical network and/or virtual network. A physical network can be for example a physical telecommunications network connecting numerous computing nodes or system such as computer servers and computer clients. A virtual network can for example, combine numerous physical networks or parts thereof into logical virtual network. In another example, numerous virtual networks can be defined over a single physical network. Implant system 100, local system 200, artificial sensory system 300, and remote system 400 can be computing node-based devices. In one embodiment, each of implant system 100, local system 200, artificial sensory system 300, and remote system 400 can be external to one another. In one embodiment, local system 200, artificial sensory system 300, and remote system 400 can be co-located with implant system 100. While, in one embodiment, all processing circuitry referenced herein can be incorporated into implant system 100, local system 200, in one embodiment, can be provided to be external to implant system 100. In one embodiment, local system 200 can be provided, e.g., by a laptop, smartphone, personal computer, and the like. Embodiments herein recognize that while components including processing circuitries co-located in implant system 100 can be expanded, it can also be advantageous in some embodiments to distribute such components including processing circuitries, e.g., for heat and size reduction.

The neocortex is the thin outer layer of the cerebrum and features a 6-layer horizontal laminar organization (nomenclature: layers I-VI). Groups of cells are organized into two-dimensional maps across the surface of the neocortex into neuronal groupings called ‘columns’; these columns are perpendicular to the surface of the neocortex and project vertically across the six layers of cortex and serve as the basic structural motif of the neocortex. Neuronal inputs from the thalamus innervate Layer IV (the granular layer), which typically leads to both infragranular (Layers V-VI) and supragranular (Layers I-III) signal processing (Buxhoeveden and Cadanova 2002). Attributes of system 1000 herein are described with reference to scenarios in which system 1000 interacts with a neocortex, at least part of which has been photonically enabled to be responsive to light emissions as set forth herein. The light emissions can include light emissions directed to areas genetically altered to be responsive to light emissions, e.g., areas including and about the neocortex, and can include light emissions directed to inputs to the neocortex.

System 1000 described can emit light with use of emitter array 140 to stimulate thalamic inputs that terminate in Layer IV, after the thalamic inputs have been altered genetically to be reactive to light. To calibrate stimulation power levels from the prosthetic implant, the neurons of the cortex can be altered transgenically to emit light either bioluminescently or fluorescently when driven by the implant's emitters. This will allow the implant system to remain in constant calibration through a real-time control loop as will be further described herein. In one embodiment, the thalamic inputs to be stimulated are LGN neurons that terminate at Layer IV of primary visual cortex (V1). Detectors receive bioluminescently or fluorescently emitted photons from neurons in the primary visual cortex portion of neocortex that have been modified transgenically to produce light-emitting or fluorescent proteins that vary in their emission as a function of activity level.

Regarding circuit map and organization features of the primary visual cortex (V1), thalamic inputs from the lateral geniculate nucleus (LGN) innervate Layer IV cortical neurons. Columns are specialized and arranged into structures called hypercolumns in V1. A hypercolumn consists of four tuned columns, all of which are related by the fact that they all are sensitive to the same visual point in space (though each to different aspects of visual perception)—which individually will be referred to as hypercolumn quadrants for this patent. Each hypercolumn quadrant provides information on light or dark stimulus (sign of contrast) arriving from each eye (i.e., ON signal from left eye, ON signal from right eye, OFF signal from left eye, OFF signal from right eye). A hypercolumn serves as the fundamental structure that processes all of the information of the smallest region of space that a person can visually perceive. Thus, by controlling the activity of individual hypercolumns precisely, the prosthetic will provide artificial visual perception at the highest attainable resolution of natural vision.

Implant system 100 can be responsible for activating a cortical column array 8 represented by cortical map 10 of user 20 defined by the user's neocortex. In some embodiments, the part of neocortex interacted with by system 1000 may be the primary visual cortex (V1), primary auditory cortex (A1), primary somatosensory cortex (Si) or another cortical region of neocortex. In the primary visual cortext, the cortical column array 8 can be defined by an array of hypercolumns (a cortical hypercolumn array). Cortical column array 8 represented by cortical map 10 can be defined by organized columns. In one embodiment, cortical column array 8 represented by cortical map 10 can include hypercolumns defined by organized columns called hypercolumn quadrants. User 20 in one use case can be a sensory impaired or sensory deprived user or in another use case can be a normally abled user. In one embodiment, User 20 can be a vision impaired or blind user or in another use case can be a sighted user. User 20, which is acted on by system 1000 can be, e.g., a human or other organism. In one embodiment, implant system 100 can include emitter array 140. With use of emitter array 140 system 1000 can present external stimulus data to a user's cortical map. In one embodiment, external stimulus may be scene image. In another embodiment, external stimulus may be an auditory stimulus e.g., sound; somatosensory stimulus e.g. mechanical force, temperature change, etc.; olfactory stimulus e.g. chemical stimuli; or other external stimulus. In the case of sensory restoration, the external stimulus data can be selected to replicate a field of view of a normally sensory abled user. In an embodiment, the sensory restoration is vision restoration where the external stimulus data is scene image data that can be selected to replicated field of view of a normally sighted user. In other applications, the external stimulus data can be any arbitrary external stimulus data such as a scene from any remote location relative to user 20, or from the output of a handheld or other connected device. Emitter array 140 can include, in one embodiment, a plurality of emitters arranged in a grid pattern.

Embodiments herein recognize with reference to FIG. 1B that a sensory system of a user can include a sensory tissue onto which focused external stimulus from a live environment is focused, a pathway from the sensory tissue to a thalamic station, and a set of said thalamic station neurons that connect to layer IV of a user's primary visual cortex (V1) at cortical column array 8 represented by cortical map 10. In an embodiment, with reference to FIG. 1B, the sensory tissue may be the retina, the external stimulus may be light from a live visual scene, the thalamic station may be the lateral geniculate nucleus (LGN) whose neuronal terminals connect to layer IV of the primary visual cortex (V1) of cerebral cortex. Embodiments herein can bypass components of a user's sensory system to present external stimulus data to a user's cortical column array 8 represented by cortical map 10 with use of emitter array 140. Embodiments herein as will explained in further detail can include activation of neurons that are subject to preparation by being made light sensitive through use of optogenetics. In one embodiment, neurons that input the sensory information into the neocortex, e.g., thalamic projections, can be made light-sensitive through a gene therapy tool called “optogenetics” and those neurons will be activated by light emitters of emitter array 140 to bypass natural inputs from the sensory tissue.

Embodiments herein recognize with reference to FIG. 1B that a natural vision system of a user can include a retina 24 onto which focused light from a live scene is focused, a pathway 26 from the retina to a lateral geniculate nucleus (LGN) 28, and a set of LGN neurons 30 that connect to layer 4 of a user's primary visual cortex (V1) at cortical column array 8 represented by cortical map 10. Embodiments herein can bypass components of a user's vision system to present image data to a user's cortical column array 8 represented by cortical map 10 with use of emitter array 140. Embodiments herein as will be explained in further detail can include activation of neurons that are subject to preparation by being made light sensitive through use of optogenetics. In one embodiment, neurons that input the visual information into the neocortex, e.g., thalamic projections, can be made light sensitive through a gene therapy tool called “optogenetics” and that those neurons will be activated by light emitters of emitter array 140 to bypass natural inputs from the retina 24.

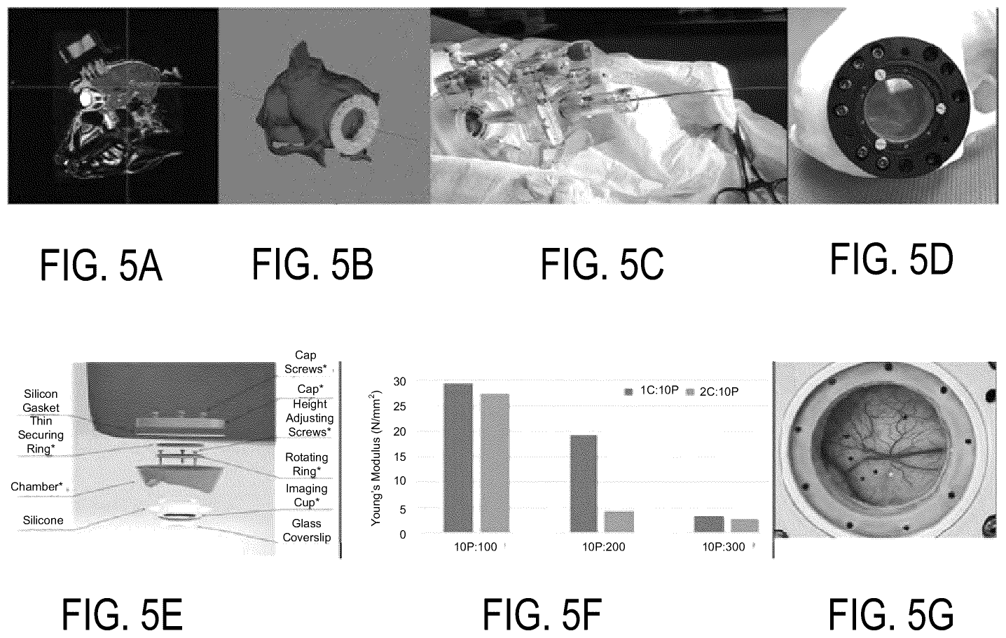

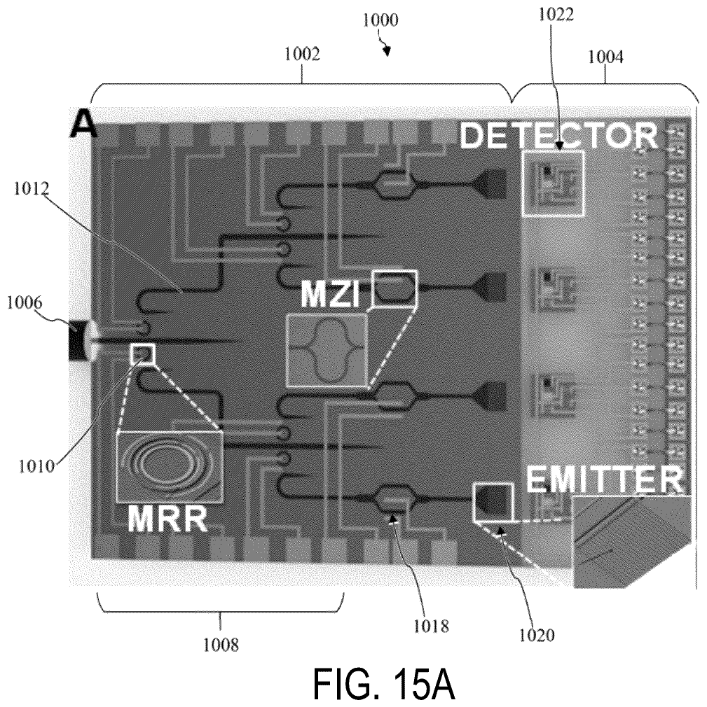

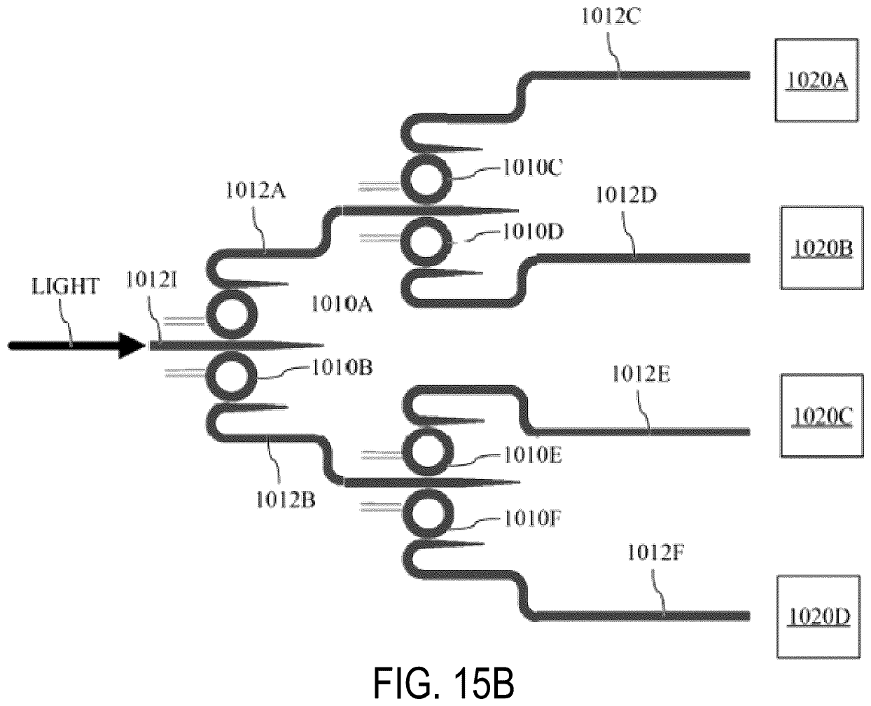

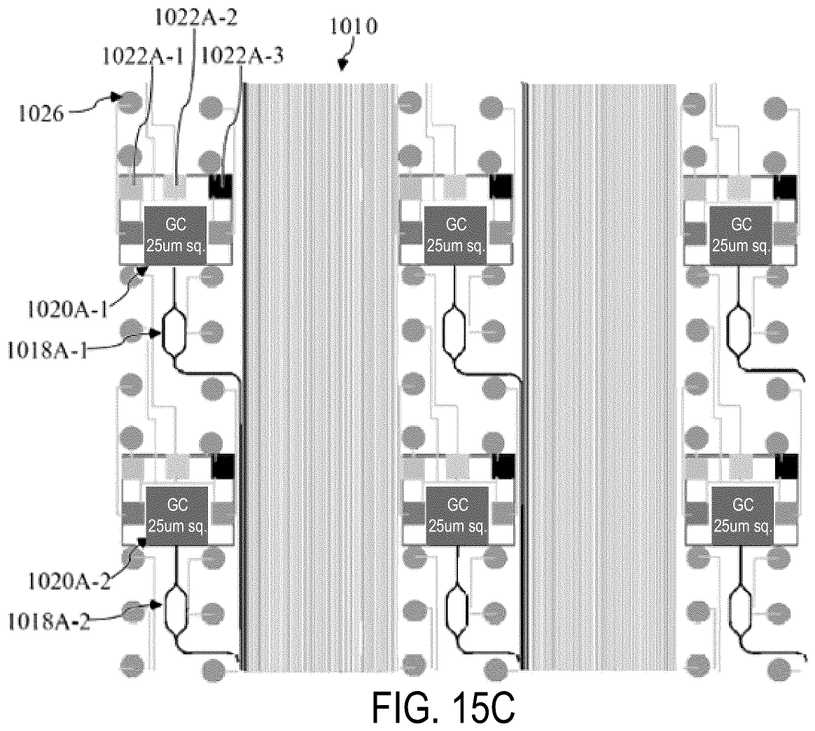

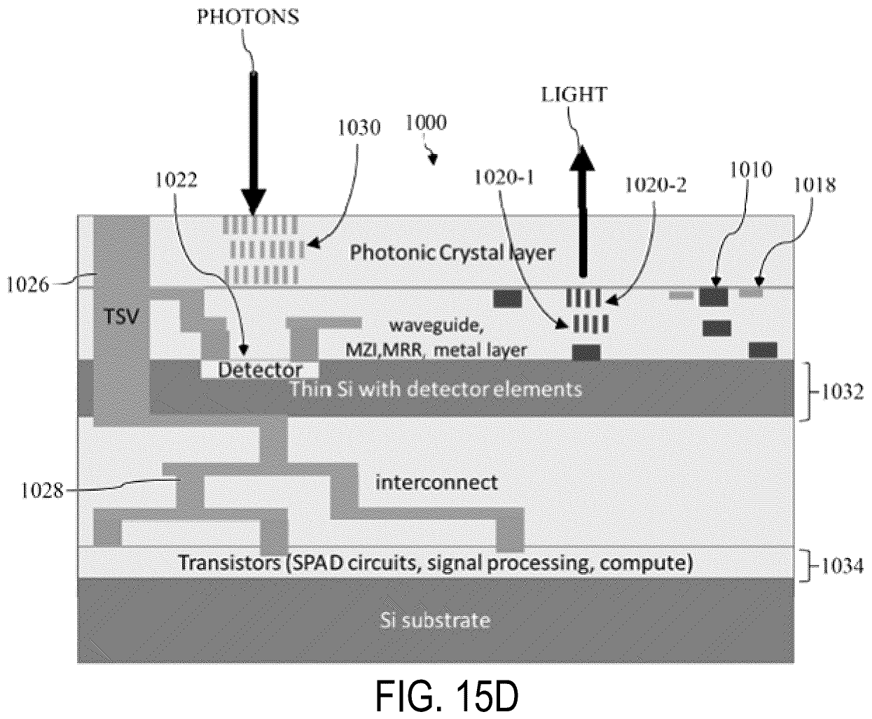

One or more of (and in one embodiment each of) emitter array 140, detector array 150, and components 110, 115, 120, 130, 140, 138, 148, 180 can be co-located in implant system 100. Implant system 100 (implant) can have a physical housing defining its exterior as depicted in FIG. 1A and FIGS. 5A-5E and can be adapted to be implanted in user 20 and in one use case can be adapted to be positioned on the surface of the cortex of user 20 over the foveal retinotopic representation of the visual field in the primary visual cortex, according to one embodiment. One or more of (and in one embodiment each of) emitter array 140, detector array 150, and components 110, 115, 120, 130, 140, 138, 148, 180 can be disposed in the housing. The housing, as shown in FIGS. 5A-5D can be defined, e.g., by a chamber and an imaging window for transmission of emit and receive radiant energy. The implant can be adapted to be disposed in user 20 by being adapted to be at least partially disposed in user 20, e.g., in an area in, on, about a brain of the user 20, as described by the exemplary positioning of the implant. Alternative embodiments of hardware defining implant system 100 (implant) including emitter array 140 and detector array 150 are set forth throughout the description including with reference to FIGS. 15A-15D which sets forth an example of an emitter assembly defining emitter array 140 and an example of detector assembly defining detector array 150. In one example, emitters of emitter array 140 can be provided by emitter devices as set forth in reference to FIGS. 15A-15D, and detectors of detector array 150 can be provided by detector devices as set forth in reference to FIGS. 15A-15D.

In one embodiment, the pitch of emitters defining emitter array 140 can be selected to be coordinated with the pitch of columns defining cortical column array 8 represented by cortical map 10. In one embodiment, the columns may be hypercolumn quadrants. In one embodiment, the pitch of detector array 150 can be coordinated in dependence on a pitch of columns defining cortical column array 8 represented by cortical map 10. In one embodiment, a pitch of emitter array 140 can be selected to be less than a pitch of columns defining cortical column array 8 represented by cortical map 10. Selecting a pitch of emitters defining emitter array 140 to be less than a pitch of columns defining cortical column array 8 represented by cortical map 10 can increase the likelihood of there being at least one emitter for activating respective columns of a neocortical area associated to implant system 100. Areas herein can refer to volumetric areas except to the extent the context indicates otherwise.

In one embodiment, the pitch of emitters defining emitter array 140 can be selected to be coordinated with the pitch of hypercolumn quadrants defining cortical column array 8 represented by cortical map 10. In one embodiment, the pitch of detector array 150 can be coordinated in dependence on a pitch of hypercolumn quadrants defining cortical column array 8 represented by cortical map 10. In one embodiment, a pitch of emitter array 140 can be selected to be less than a pitch of hypercolumn quadrants defining cortical column array 8 represented by cortical map 10. Selecting a pitch of emitters defining emitter array 140 to be less than a pitch of hypercolumn quadrants defining cortical column array 8 represented by cortical map 10 can increase the likelihood of there being at least one emitter for activating respective hypercolumn quadrants of a V1 area associated to implant system 100.

Implant system 100 in one embodiment can include detector array 150. Detector array 150 can include a plurality of detectors in a grid pattern. Detector array 150 can be selected to have a pitch less than a pitch of columns defining cortical column array 8 represented by cortical map 10 in order to increase the likelihood of there being at least one dedicated detector for detecting light emissions from each respective column defining cortical column array 8 represented by cortical map 10. In one embodiment, the columns are hypercolumn quadrants of a hypercolumn. Detector array 150 can be selected to have a pitch less than a pitch of hypercolumn quadrants defining cortical column array 8 represented by cortical map 10 in order to increase the likelihood of there being at least one dedicated detector for detecting light emissions from each respective hypercolumn quadrant defining cortical column array 8 represented by cortical map 10.

Each of implant system 100, local system 200, and artificial sensory system 300 can include one or more processor 110, one or more working memory 120, e.g., RAM, one or more storage device 130, and one or more communication interface 180. The one or more processor 110, one or more working memory 121, one or more storage device 130, and one or more communication interface 180 can be connected and in communication via system bus 115. Each of implant system 100, local system 200, and artificial sensory system 300 can also include I/O devices 140 connected to system bus 115. Examples of I/O devices 140 include but are not limited to microphones, speakers, Global Positioning System (GPS) devices, cameras, lights, accelerometers, gyroscopes, magnetometers, sensor devices configured to sense light, proximity, heart rate, body and/or ambient temperature, blood pressure, and/or skin resistance, a keyboard, a keypad, a pointing device, a display, and/or any other devices that enable a user to interact with computer system 500 and activity monitors.

Implant system 110, in addition to the components 110, 120, 130, 180, and 115 can include emitter array 140 connected to system bus 115 via interface 138 and detector array 150 connected to system bus 115 via interface 148. Artificial sensory system 300, in addition to the components 110, 120, 130, 180, and 115 can include scene camera image sensor 160 and eye tracking camera image sensor 170. Scene camera image sensor 160 can be in communication with system bus 115 of artificial sensory system 300 via interface 158 and eye tracking camera image sensor 170 can be in communication with system bus 115 of artificial sensory system 300 via interface 168. Artificial sensory system 300 can include one or more additional sensor 164 connected via interface 162 to system bus 115 for sensing sensory information. The one or more additional sensor can include, e.g., an auditory stimulus e.g. sound sensor; somatosensory stimulus e.g. mechanical force sensor, temperature change sensor, olfactory stimulus e.g. chemical stimuli sensor; or other external stimulus sensor.

Local system 200, in one embodiment, can include, e.g., a keyboard and display to facilitate, e.g., input of control data and display of result data. The providing of local system 200 external to implant system 100 can facilitate removal of heat from an area proximate a user's neocortex.

Implant system 100, in one embodiment, is not used as an implant but is an integrated co-planar image sensor or camera and image display screen. Emitter array 140 can be selected to have a pitch optimal for a desired pixel resolution of the displayed image. Implant system 100 in one embodiment can include detector array 150 to capture an image viewed by the detector array surface. In one aspect, the detector array serves as a camera, detecting a local visual scene, while displaying an image to a user, effectively integrating an image display screen with a camera or image capture device.

Referring to user 20 as shown in FIG. 1A, user 20 can be wearing artificial sensory system 300 with supporting external apparatus. In an embodiment, the artificial sensory system 300 that user 20 wears may be a sensory system for input of artificial vision sensory information or other types of sensory information, e.g., external stimulus may be an auditory stimulus e.g. sound; somatosensory stimulus e.g. mechanical force, temperature change, etc; olfactory stimulus e.g. chemical stimuli; or other external stimulus. The selected pitch of emitter array 140 and detector array 150 can be provided to facilitate fine grain activation of columns that control sensory perception by providing emitter array 140 to have emitters that can activate columns on one emitter per column, or approaching a one (or more) emitter per column basis—while reducing crosstalk between column activations-artificial sensation or perception can approach a resolution of natural sensation or perception. With use of a combination of power and pulse-width modulated light-based stimulation of columns, photonic input into a neocortical area can be limited to avoid damage to brain tissue of a user's neocortical area. In one embodiment, referring to the view of user 20 as shown in FIG. 1A, user 20 can be wearing artificial sensory system 300 having eyewear frame 302, e.g., provided by spectacles (glasses) frame. The selected pitch of emitter array 140 and detector array 150 can be provided to facilitate fine grain activation of hypercolumn quadrants that control human vision by providing emitter array 140 to have emitters that can activate hypercolumn quadrants on a one emitter per hypercolumn quadrant, or approaching a one (or more) emitter per hypercolumn quadrant basis—while ensuring minimized crosstalk between hypercolumn quadrants during activation of individual quadrants-artificial vision can approach a spatial resolution of natural vision. With use of a combination of power and pulse-width modulated light-based stimulation of hypercolumn quadrants, photonic input into a primary visual cortex (V1) can be limited to avoid damage to brain tissue of a user's primary visual cortex (V1).

Referring to FIG. 1A, eyewear frame 302 of artificial sensory system 300 can support scene camera image sensor 160 (one for each eye) and eye tracking camera image sensor 170 (one for each eye). The field of view indicated by Θ of scene camera image sensor 160 can encompass spatial subject matter forward of user 20 to replicate the field of view of a sighted person. The field of view indicated by a of eye tracking camera image sensor 170 can encompass an eye of user 20 so that video data captured using eye tracking camera image sensor 170 can include a representation of eye position changes by user 20. As the eye position changes in each eye, the eye tracking camera image sensor 170 can relay that information to local system 200, which will also collect with use of scene camera image sensor 160 images and determine which pixels of the scene should be stimulated into the specific corresponding hypercolumn quadrants in a visual cortex.

System 1000 can subject such captured eye-representing image data to video image recognition processing to discern changing positions of the eye of a user 20 over time, and then can use such information to adjust image data presented to a user by control of photonic emissions by emitter array 140 in order that a scene viewed by user 20 can be controlled by eye movements of the user 20 replicating an aspect of natural vision.

In one aspect, implant system 100 can be configured to emit light at specific point locations of a user's cortical column array 8 represented by cortical map 10 in order to selectively activate columns defining cortical column array 8 represented by cortical map 10. Detector array 150 of implant system 100 can, in one aspect, detect whether controlled emitters are properly stimulating columns to which they are intended to stimulate. In one aspect, signals produced by the cortex can be detected by the detector array 150 and used to determine which of emitters of emitter array 140 are aligned or not aligned to a specific column of cortical column array 8 represented by cortical map 10. Misaligned emitters can be disabled in order to reduce crosstalk between columns and also reduce power consumption and heat emissions potentially dangerous to user 20. In one aspect, implant system 100 can be configured to emit light at specific point locations of a user's cortical column array 8 represented by cortical map 10 in order to selectively activate hypercolumn quadrants defining cortical column array 8 represented by cortical map 10 of the primary visual cortex (V1). Detector array 150 of implant system 100 can, in one aspect, detect whether controlled emitters are properly stimulating hypercolumn quadrants to which they are intended to stimulate. In one aspect, signals produced by the cortex can be detected by the detector array 150 and used to determine which of emitters of emitter array 140 are aligned or not aligned to a specific hypercolumn quadrant of cortical column array 8 represented by cortical map 10. Misaligned emitters can be disabled in order to reduce crosstalk between hypercolumn quadrants and also reduce power consumption and heat emissions potentially dangerous to user 20. Crosstalk between hypercolumn quadrants can be perceived by the user as visual glare, and as with true optical glare, it degrades visual perception. Also, by increasing the density of the emitter array 140 and then disabling the emitters of emitter array 140 that are not aligned to individually targeted hypercolumn quadrants, system 1000 can decrease noise and increase spatial resolution by increasing the accuracy with which spatial information is presented to user 20.

Signals detected by the detector array 150 can also be used to regulate power delivery levels of emitted light into the cortical column array 8 represented by cortical map 10. Power delivery level of light emission of an emitter can be controlled e.g., with use of power amplitude control and/or with on time control, e.g., pulse width modulation. By regulating power delivery levels, e.g., with use of power amplitude control and/or temporal pulse widths associated to emitter emissions, power consumption and heat imposed to brain tissue of a neocortex can be further reduced. In some use cases, implant system 100 by controlling power level associated to respective ones of emitters of emitter array 140 can improve accuracy with which image data is presented to a user photonically via emissions by emitter array 140. In one embodiment implant system can present differentiated gray levels to a cortical column array 8 represented by cortical map 10 with use of different power levels. In an aspect, the part of neocortex being interfaced may be primary visual cortex.

Artificial sensory system 300 can generate streaming video data including streaming video data representing a scene forward of user 20 having field of view, Θ, with use of scene camera image sensor 160 and also user eye representing streaming video data representing movements of a user eye with use of eye tracking camera image sensor 170. Streaming video data produced with use of scene camera image sensor 160 can be input into a user's cortical column array 8 (defining by cortical hypercolumn array in the visual cortex) represented by cortical map 10 with use of emitter array 140. Streaming video data produced using eye tracking camera image sensor 170 can be processed to ascertain a current position of a user's eye, e.g., which can be indicative of direction which user 20 is currently looking at. System 1000 can use the described eye position data in order to determine a portion of a frame of image data and a set of streaming image data to present to a user.

In one embodiment, implant system 100 and artificial sensory system 300 can be in communication with local system 200 via the respective one or more communication interface 180 of implant system 100, artificial sensory system 300, and local system 200.

Remote system 400 can store various data and can perform various functions. Remote system 400, in one embodiment, can be provided by a computing node based system hosted within a multi-tenancy computing environment hosting remote system 400, and, in one embodiment, can have attributes of cloud computing as defined by the national Institute of Standards and Technology (NIST). Remote system 400, in one embodiment, can include data repository 408 for storing various data. Data stored within data repository 408 can include, e.g., calibration parameter values that define a calibration process. Remote system 400 can run one or more process 411. The one or more process 411 can include, e.g., a video conferencing process in which user 20 participates in a video conference with a remote user. The videoconferencing process can facilitate the presentment to user 20 by way of light emissions by emitter array 140 into cortical column array 8 represented by cortical map 10 image data defined by remote video data including live remote video data from locations external to a current location of user 20.

Data repository 408 of remote system 400 can also include various video data files which can be run for playback and insertion of video streaming data into cortical column array 8 represented by cortical map 10 with use of emitter array 140.

System 1000 can include data repository 1080 defined by working memories and storage devices of implant system 100, local system 200, and artificial sensory system 300, and by data repository 408 of remote system 400. System 1000, e.g., by implant system 100 and/or local system 200, can run various processes.

System 1000 running calibration process 111 can include system 1000 performing a calibration so that select ones of emitters of emitter array 140 are enabled and select ones of emitters of emitter array 140 are disabled. System 1000 running calibration process 111 can configure implant system 100 so that radiant energy imposed to brain tissue by implant system 100 is reduced and further so that spatial resolution of emitted image data emitted by implant system 100 is increased.

System 1000 running power regulating process 112 can include system 1000 emitting light into cortical column array 8 represented by cortical map 10 using emitter array 140. System 1000 running power regulating process 112 can include system 1000 emitting light into cortical column array 8 represented by cortical map 10 using emitter array 140 and detecting a response signal using detector array 150. System 1000 running power regulating process 112 can include system 1000 controlling energy delivery input in emitted light in dependence on response signal information as detected by detector array 150. Power delivery for emission of light can be controlled so that light emissions do not exceed power delivery level suitable for stimulation of a hypercolumn quadrant. In such manner, risk imposed to tissue defining a visual cortex (V1) delivery of radiant energy can be reduced.

System 1000 running scene selection process 113 can include system 1000 adjusting pixel positions defining streaming frames of video data controlling image data presented to a user photonically via light emissions by emitter array 140 into cortical column array 8 represented by cortical map 10. In one aspect, pixel positions controlling image data presented to a user defining a frame of image data in a set of frames defining streaming video image data can be selected in dependence on a current eye viewing direction of a user. Eye viewing directions can include the directions, e.g., central gaze, and positions varying from a central gaze position that can be expressed in terms of vertical eye position and/or horizontal eye position.

In one aspect, system 1000 can be configured so that when an eye of user 20 is determined to be at a central gaze position looking straight ahead by processing of streaming video data produced using eye tracking camera image sensor 170, a subset of positions provided by center pixel positions defining streaming video data frames of image data can be selected for controlling light emissions by emitter array 140 to cortical column array 8 represented by cortical map 10 of a user 20. However, when a user, by processing of streaming video data obtained using eye tracking camera image sensor 170, is determined to be looking left, the selected subset of pixel positions can be shifted leftward and correspondingly, when the user is determined to be looking right, the selected subset of pixel positions can be shifted rightward and correspondingly, when the user is detected to be looking up, the pixel positions can be shifted upward, and correspondingly, when the user detected to be looking down, the pixel positions can be shifted downward. In such manner, a scene defined by pixel image data viewed by a user by activation of cortical column array 8 represented by cortical map 10 using emitter array 140 can be controlled with eye movements of the user thus emulating natural vision.

FIG. 1C depicts cortical column array 8 represented by cortical map 10 defined by columns 11 of a user's neocortex. In the embodiment exemplified in 1C, the columns are hypercolumn quadrants 11 of a user's primary visual cortex (V1) of the neocortex. In the embodiment exemplified, FIG. 1C depicts a schematic physiological representation of cortical column array 8 represented by cortical map 10 while FIG. 1D depicts a functional schematic representation of a cortical map 10. Referring to cortical column array 8 represented by cortical map 10, embodiments herein recognize that all external sensory stimulus, regardless of degrees of freedom, are mapped and organized on the 2-dimensional surface of the respective sensory-processing neocortical region. External sensory stimulus would normally stimulate sensory tissue, which would transduce a signal to its respective thalamic station; the signal is relayed from thalamic station neuronal terminals to layer IV of neocortex to stimulate the columns of the cortical map. Embodiments herein recognize that activation of these columns in the correct spatiotemporal pattern corresponds to sensory stimulation of spatial portions of cortical column array 8 represented by cortical map 10 that would have been activated by normal sensory stimulation in a healthy normally abled individual. This activation will thus be drivable by external stimulus detected data-once that data is processed so as to represent the processing done by the sensory system between the sensory tissue and thalamic station. This will restore sensory perception prosthetically.

Embodiments herein recognize that cortical column arrays 8 can be represented by cortical maps. One example is cortical map 10 as shown in FIG. 1D for a cortical column array 8, wherein the cortical column array is a hypercolumn cortical column array of a visual cortex. FIGS. 1C and 1D depict cortical column array 8 represented by cortical map 10 defined by subcortical inputs to hypercolumn quadrants 11 of a user's primary visual cortex (V1). FIG. 1C depicts a schematic physiological representation of cortical column array 8 represented by cortical map 10 while FIG. 1D depicts a functional schematic representation of a cortical map. Referring to cortical column array 8 represented by cortical map 10, embodiments herein recognize that spatial portions of cortical column array 8 represented by cortical map 10 map to points in image space that are normally imaged by a retina during normal viewing. That is, the relationship between regions on the cortical surface are organized in the same gross layout as the retina—it is a retinotopic map, which can be regarded to be a cortical retinotopic map. Because the retina is an optical device its organization follows from the visual world. A given point on a retinotopic map, including a cortical retinotopic map represents a point in space, and the adjacent abutting position directly to one side abuts the corresponding retinal position in the retinotopic map, whereas the opposite abutting position has a correspondingly opposite position on the retinotopic map. The logic follows just as it would for the optical image that is projected onto a digital camera CCD or CMOS sensor by the camera's lens. The same logic follows furthermore in the cortex, where cortical column array 8 represented by cortical map 10 defines a cortical retinotopic map and wherein there is a one-to-one correspondence of positions in the visual world laid out like pixels on a video screen. Each individual position in the cortical column array 8 represented by cortical map 10 defining a cortical retinotopic map represents all of the visual information about that position region known as a “hypercolumn.” Embodiments herein recognize that each hypercolumn's inputs from the retina-via the lateral geniculate nucleus (LGN) of the thalamus—has four input quadrants, one quadrant each to encode bright visual stimuli in the left and right eyes, and one quadrant each to encode dark visual stimuli in the left and right eyes.

Embodiments herein recognize that activation of these hypercolumn quadrants in the correct spatiotemporal pattern corresponds to visual stimulation of spatial portions of cortical column array 8 represented by cortical map 10 that are activated by normal visual stimulation in a healthy sighted individual. Embodiments herein recognize that this activation can thus be drivable by a scene camera's pixel data-once that data is processed so as to replace and bypass the processing done by the visual system between the retina and LGN. The described system can stimulate artificial vision prosthetically.

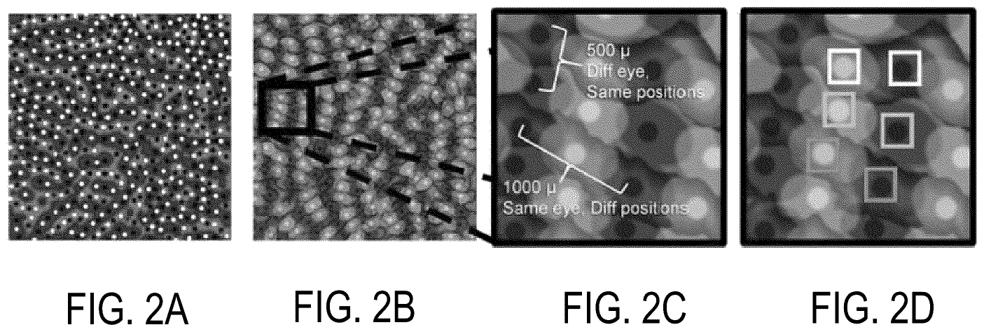

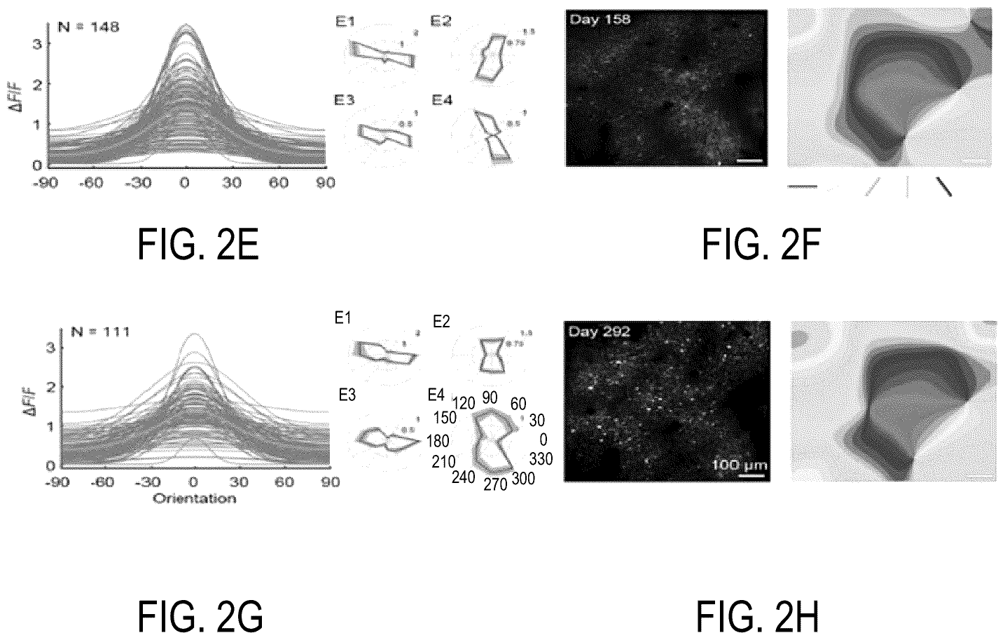

In one aspect, spatial portions of cortical column array 8 represented by cortical map 10 can be logically divided into these cortical hypercolumns (the visual system's pixels 12) that define a grid of retinotopic positions, e.g., pixel positions A1-G7 of hypercolumns 12 that can be regarded as cortical pixels in the pixel map provided by cortical column array 8 represented by cortical map 10 of FIG. 2. In one aspect, each cortical hypercolumn 12 of cortical column array 8 represented by cortical map 10 defining a cortical retinotopic map, can be defined by first, second, third, and fourth hypercolumn quadrants where the top left hypercolumn I can be a left eye light (ON) quadrant for that retinal position, the top right hypercolumn quadrant II can be a right eye light (ON) hypercolumn, the lower left hypercolumn quadrant III can be the left eye dark (OFF) hypercolumn quadrant, and the lower right hypercolumn quadrant IV can be the right eye dark (OFF) hypercolumn quadrant.

Embodiments herein recognize that in normal visual perception, the left ON hypercolumn quadrant in this example can be stimulated when the corresponding position of the left eye's retina is illuminated by a light spot. Embodiments herein further recognize that illumination of a spot on the right retina (at the same position in visual space) results in right eye visual perception when the right ON hypercolumn quadrant is stimulated. Moreover, the visual image of a dark spot at the same position in the left retina can result in visual perception of a dark spot when stimulation of the left OFF hypercolumn quadrant occurs, and the same dark spot at the same retinal position in the right eye can be seen as a dark spot in the right eye when it results in stimulation of the right OFF hypercolumn quadrant. Expanding the visual stimulus set from small spots at the resolution limit of vision, now using different levels of visual contrast and different visual objects having different sizes and shapes, results in varied activation patterns of hypercolumn quadrant inputs to result in the entire gamut of visual experience. Replicating this same pattern prosthetically will result in equivalent prosthetic perception.

Embodiments herein recognize that hypercolumns of cortical column array 8 represented by cortical map 10 can be prosthetically stimulated to artificially stimulate vision to a user by way of the appropriate emissions of light by emitter array 140. The artificially stimulated vision can be provided to vision impaired or blind user, or to a sighted user who enjoys normal vision, for the purpose of artificially augmented vision. In one aspect, emissions of light by emitter array 140 for presentment of image data to cortical column array 8 represented by cortical map 10 can be provided in dependence on pixel data of a video frame of image data provided by a camera image sensor, such as a scene camera image sensor 160 as shown in FIG. 1A having the field of view, Θ.

Referring to Fig. D, for example, the center hypercolumn 12 providing a cortical pixel at pixel position D4 can be activated so that user 20 can observe a binocular dark spot by appropriate stimulation from the emitter elements corresponding to the two OFF hypercolumn quadrants within the cortical hypercolumn. For causing the user to see a binocular light spot, an emitter that activates hypercolumn ON quadrants I and II (left and right) can be controlled in its power delivery level, e.g., with use of emission amplitude control and/or pulse width modulation to control the amount of excitation, which will vary the amount of brightness of the perceived prosthetic perception. Correspondingly, emitters that activate OFF hypercolumn quadrants III and IV (left and right) will control the perceived darkness of the prosthetic perception by varying emission power and/or pulse width modulation. By combining activation of ON and OFF hypercolumn quadrants in each eye, full binocular and stereoscopic control of contrast perception can be achieved, enabling user 20 to see dark, light, or gray spots at the highest obtainable acuity by activating the corresponding emitters for hypercolumn quadrants in the corresponding position of the visual field. By orchestrating stimulation patterns of hypercolumn quadrants that follow from what would occur through activation in sighted persons during natural visual perception, the prosthetic device will restore vision in all of these domains to perceive any object or scene in the world. As such, the described process for cortical hypercolumn pixel D4 can be applied to a multitude of cortical hypercolumns in the cortical column array 8 represented by cortical map 10 so that user 20 is stimulated prosthetically to see an image defining a scene, e.g., the scene within a field of view, Θ, of scene camera image sensor 160.

Referring now to FIG. 1E, a configuration for emitter array 140 and detector array 150 is described. In one aspect, as indicated in FIG. 1E, emitters are represented by triangles and detectors are represented by squares-arranged in an interleaved fashion so that each emitter of emitter array 140 has a plurality of neighboring detectors and each detector of detector array 150 has a plurality of neighboring emitters. In one aspect, emitter array 140 and detector array 150 can be configured to have densities larger (smaller pitch spacing) than a density of hypercolumn quadrants defining cortical column array 8 represented by cortical map 10. Densities of emitter array 140 and detector array 150 can be expressed in terms of pitch, i.e., center spacing between emitters in the case of emitter array 140A and center spacing distance between detectors in the case of detector array 150. As set forth herein emitter array 140 and detector array 150 can be regarded to have pixel resolutions indicated by the pixel positions A1-G7 that map the pixel positions of cortical map 10. In the illustrative embodiment of FIG. 1E, emitter array 140 and detector array 150 can define a 7×7 grid of pixel positions. Emitter array 140 and detector array 150 can be scaled up according to requirements of an application in order to facilitate emissions and response signal detections of a larger area of a cortical column array 8 provided by a cortical hypercolumn array,

In one embodiment, emitters of emitter array 140 and detectors of detector array 150 can be configured to have respective pitches that are at one-half or less than the pitch of hypercolumn quadrants defining cortical column array 8 represented by cortical map 10 in each dimension. That is, the emitter/detector dyads can be at least 2× the density of the hypercolumn quadrants in each dimension. Since hypercolumn quadrants occur in two dimensions across the two-dimensional surface of the cortex, there can be approximately 4× the number of emitter/detector dyads as hypercolumn quadrants, so that at least one emitter/detector dyad is positioned optimally within each hypercolumn quadrant to stimulate the quadrant in isolation, without crosstalk between quadrants. In one embodiment, the pitch of emitters defining emitter array 140 and detectors defining detector array 150 can be provided to result in an emitter/detector dyad density of at least 4× the density of hypercolumn quadrants of cortical column array 8 represented by cortical map 10—with at least 16 emitter/detector dyads for every 4 hypercolumn quadrants.

Providing the density of emitter array 140 and detector array 150 to be greater than the density of hypercolumn quadrants in the cortical column array 8 represented by cortical map 10, as defined by hypercolumn quadrants 11, can increase the likelihood of each quadrant in cortical column array 8 represented by cortical map 10 being optimally stimulated by at least one emitter in the emitter array 140. Substantially, one enabled emitter for each hypercolumn quadrant of cortical column array 8 represented by cortical map 10 within a coverage area of implant system 100 and substantially one detector per hypercolumn quadrant 11 within the coverage area of implant system 100, can result in the capability of full real-time control and power/stimulation calibration of prosthetic vision at the highest obtainable acuity of visual perception.

In Table A below, there are provided exemplary pitch ranges for emitter array 140 and detector array 150. As seen from Table A, a pitch of emitter array 140 and detector array 150 can be configured to be coordinated with the pitch of hypercolumn quadrants 11 defining cortical column array 8 represented by cortical map 10.

TABLE A

| | Hypercolumn | | Emitter array | | Detector array | |
|---|---|---|---|---|---|---|
| | quadrant pitch | | 140 | | 150 | |
| Embodiment | (nominal) | | pitch range | | pitch range | |
| 1 | 100-400 | microns | 50-200 | microns | 50-200 | microns |
| 2 | 350-650 | microns | 175-325 | microns | 175-325 | microns |
| 3 | 600-900 | microns | 300-450 | microns | 300-450 | microns |
| 4 | 850-1150 | microns | 425-575 | microns | 425-575 | microns |
| 5 | 1150-1450 | microns | 575-725 | microns | 575-725 | microns |

Embodiments herein recognize that without precise control of which emitters are emitting, noisy and sometimes deleterious image data can be presented to a user via emitter array 140 to cortical column array 8 represented by cortical map 10. In one example, if an emitter intended to stimulate an ON hypercolumn quadrant is instead misaligned with an OFF hypercolumn quadrant, user 20 will misperceive a dark spot rather than the intended light spot. In another example, if an emitter is misaligned and stimulates more than one hypercolumn quadrant at a time, the resultant percept for user 20 will be a corrupted version of what was presented to the scene camera (i.e., which could result in perceived glare). In another example, if an emitter is aligned simultaneously to a plurality of hypercolumn quadrants, e.g., at the midpoint between antagonistic and mutually inhibitory hypercolumn quadrants, dark might be evoked simultaneously to light, at the same position, which can result in reduced quality or total loss of prosthetic perception.

Embodiments herein can provide for calibration process 111 (FIG. 1A) in which the alignment between emitters and hypercolumn quadrants can be discovered. By providing a density of emitted/detector dyads to be greater than a density of the underlying hypercolumn quadrants, it is expected that a significant number of emitter detector dyads can be aligned within each hypercolumn quadrant with minimized crosstalk to surrounding hypercolumn quadrants, resulting in prosthetic visual stimulation at useful acuity of the visual system. By at least doubling the density of emitted/detector dyads compared to the underlying hypercolumn quadrants in each dimension, we expect that at least one of every four emitter detector dyads will be optimally aligned within each hypercolumn quadrant without crosstalk to surrounding hypercolumn quadrants, facilitating optimized prosthetic visual stimulation at the highest obtainable acuity of the visual system. Based on such discovery, precise control of emitters of emitter array 140 can be provided to facilitate improved resolution image data emitted to user 20 with emitter array 140 with reduced noise. System 1000 by running of calibration process 111 can discover alignments between emitters of emitter array 140 and based on resulting alignment information can disable certain misaligned emitters of emitter array 140. The disabling of select emitters of emitter array 140 can reduce noise and can also reduce heat delivered to brain tissue defining the primary visual cortex (V1).

A method for performance by implant system 100 interoperating with local system 200, artificial sensory system 300, remote system 400, as well as cortical column array 8 represented by cortical map 10 is set forth in reference to FIG. 1F.

At block 1201 local system 200 can be connected to implant system 100 and responsively, implant system 100 can send identifier data identifying implant system 100 to local system 200. The identifier data can include, e.g., serial number data of the particular implant system 100 that has been implanted on a cortical column array 8 represented by cortical map 10 of user 2.

On receipt of the sent identifier data, local system 200 can proceed to calibration decision block 2201. At calibration decision block 2201, local system 200 can determine whether calibration is to be performed for calibration of the connected implant system 100. In one use case, local system 200 at block 2201 can determine by examination of identifier data that implant system 100 is a new implant system not previously calibrated and therefore at calibration block 2201 can determine that calibration is needed. In another use case, local system 200 can look up from data repository 1080 a most recent calibration of the particular implant system 100 implanted on user 20 as determined by examination of identifier data, and based on a time lapse from a most recent calibration, can determine that recalibration is needed.

Embodiments herein recognize that over time, e.g., due to physiological changes in a user's primary visual cortex (V1), movement of implant system 100, or other factors, periodic recalibration of implant system 100 can be useful. In one embodiment, local system 200 at block 2201 can determine that recalibration will proceed based on a time lapse from a most recent calibration satisfying a threshold. In another embodiment, local system 200 at block 2201 can continually assess the calibration level and adjust the use or disabling or specific emitters in real-time, e.g., performing calibration during a stimulated artificial viewing session.

On determination at block 2201 that calibration will proceed, local system 200 can proceed to block 2202. At block 2202, local system 200 can send messaging data to remote system 400 requesting remote system 400 to send updated calibration parameter values that can control a calibration process. Remote system 400 in data repository 408 can store calibration parameter values defining calibration processes. The calibration parameter values can define different processes that are mapped, e.g., to different size ranges of V1s, ages of users, and the like. Various administrator users associated to end users such as user 20, at various locations remote from remote system 400 can be uploading calibration parameter values defining calibration processes for use by all users 20 of system 1000. Remote system 400 at block 4201 can send calibration parameter values to local system 200 responsively to message data being sent at block 2202.

Responsively to the receipt of the calibration parameter values sent at block 4201, local system 200 at send block 2203 can send calibration parameter values defining a calibration process to implant system 100. Responsively to receipt of the calibration parameter values sent at block 2203, implant system 100 at emit block 1202 can commence performance of calibration process 111.

In performance of the calibration process, implant system 100, in one embodiment, may not present by emitter array 140 emissions of image data representing a scene in a field of view of a scene camera image sensor 160, but rather can send emissions defining a light pattern optimized for alignment discovery, wherein the light pattern may not represent a field of view of a scene camera image sensor 160. In an example of a calibration process, implant system 100 can perform processing to identify emitters that are aligned to particular hypercolumn quadrants defining cortical column array 8 represented by cortical map 10.

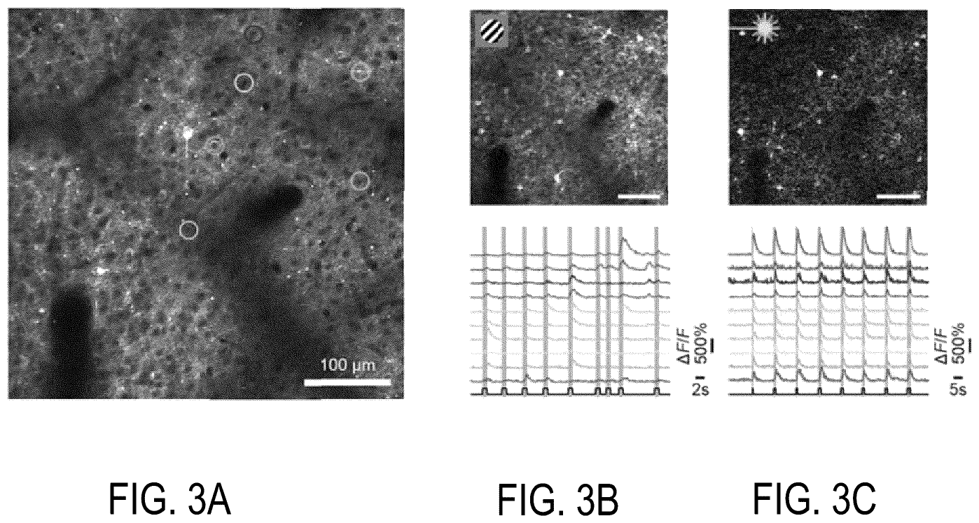

In one embodiment, implant system 100 at emit block 1202 can send emission signals to cortical column array 8 represented by cortical map 10 for discovery of at least one emitter aligned to at least one hypercolumn quadrant. In one embodiment with reference to FIGS. 2 and 3, implant system 100 at emit block 1202 can attempt to discover at least one emitter aligned to a particular hypercolumn quadrant of a hypercolumn of cortical column array 8 represented by cortical map 10.

In one embodiment, implant system 100 for discovery of a hypercolumn quadrant aligned to an emitter of emitter array 140 can control emissions of one or more emitter of emitter array 140 and can examine a response by primary visual cortex of user 20 for the presence or absence of a response signal having characteristics indicative of alignment to a hypercolumn quadrant and can examine a response by primary visual cortex of user 20 for the presence or absence of a response signal indicative of misalignment.

Referring to the flowchart of FIG. 1F, implant system 100 at emit and detect blocks 1202 and 1203 can control one or more emitter of emitter array 140 for emission of light and can detect responsive signal information transmitted at block 801 with use of one or more detector of detector array 150. For each emitter subject to emission control at block 1202 implant system 100 at block 1205 can classify the emitter as aligned, misaligned, or “insufficient information” meaning the response signal information for the emitter has been determined to be insufficient to return a classification of aligned or misaligned. At block 1206, implant system 100 can record into data repository 1080 the classification returned at block 1205 for a particular emitter and at block 1207 can ascertain whether the most recent emitter subject to classification is the last emitter subject to test by emission control at the prior iteration of emit block 1202. Implant system 100 can perform blocks 1204-1207 for each emitter subject to test during a prior iteration of emit block 1202 so that each emitter subject to test by emission control at emit block 1202 can be classified at block 1205 as being aligned, misaligned or “insufficient information”.

Implant system 100 for classifying an emitter of emitter array 140 as being aligned or misaligned can, in one embodiment, control pairs of emitters to evoke the perception of“light” “dark” ˜ or “gray” at a particular hypercolumn position (cortical pixel position) of cortical column array 8 represented by cortical map 10 of user 20, and can then detect for response signals indicating that emitters are aligned, or alternatively not aligned to a pair of hypercolumn quadrants. In one embodiment, local system 00 for performing classifying at block 1205 can examine response signal information for the signal characteristics indicative of alignment and misalignment as summarized in Table B

TABLE B

| | | |
|---|---|---|
| Emission control | Targeted return signal | Exemplary return signal |
| | characteristic if emitter A | characteristics if emitter A is |
| | is aligned to the Left-eye | misaligned to be directly |
| | ON quadrant of a | between (equally stimulating) |
| | hypercolumn and emitter | the Left-eye ON and OFF |
| | B is aligned to the Left- | quadrants, whereas emitter |
| | eye OFF quadrant of the | B is aligned to the Left-eye OFF |
| | hypercolumn. | quadrant of the hypercolumn. |
| Emitter array is commanded | The primary visual cortex | The primary visual cortex's |
| to evoke the perception of | will shine brightly with | response will be nullified as |
| lightness in the Left eye | photonic responses at the | the stimulation of the ON |
| at a targeted cortical | position of the Left-eye | emitter is misaligned and |
| retinotopic position on | ON quadrant due to the | thus will equally stimulate |
| the hypercolumn by energizing | strong stimulation and lack of | the two mutually inhibitory |
| emitter A to stimulate a left | inhibitory suppression from | ON and OFF Left eye quadrants. |
| eye ON hypercolumn quadrant | the antagonistic OFF quadrant. | The User's percept will be |
| and deenergizing emitter B | The User's percept will be | gray (no stimulus). |
| to avoid stimulating a left | Left-eye lightness at this | |
| eye OFF hypercolumn quadrant. | retinotopic position. | |
| Emitter array is | The primary visual cortex | The primary visual cortex |
| commanded to evoke | will shine brightly with | will shine brightly with |
| the perception of | photonic responses at the | photonic responses at the |
| darkness in the Left eye | position of the Left-eye | position of the Left-eye OFF |
| at the targeted cortical | OFF quadrant due to the | quadrant due to the strong |
| retinotopic position on | strong stimulation and | stimulation and lack of |
| the hypercolumn by | lack of inhibitory | inhibitory suppression from |
| deenergizing emitter A | suppression from the | the antagonistic ON |
| to avoid stimulating a | antagonistic ON quadrant. | quadrant. The User's |
| left eye ON | The User's percept will be | percept will be Left-eye |
| hypercolumn quadrant | Left-eye darkness at this | darkness at this retinotopic |
| and deenergizing | cortical retinotopic position. | position. |
| emitter B to stimulate a | | |
| left eye OFF | | |
| hypercolumn quadrant. | | |
| Emitter array is | The primary visual | The primary visual cortex's |
| commanded to evoke | cortex's response will be | response will be nullified |
| the perception of gray | nullified as the stimulation | over the Left-eye ON |
| in the Left eye at the | of the ON emitter is | quadrant position and will |
| targeted cortical | nullified by the equally | be weakened but present |
| retinotopic position on | strong stimulation of the | from the Left-eye OFF |
| the hypercolumn by | mutually inhibitory OFF | quadrant. The User's |
| energizing emitter A to | Left eye quadrant. The | percept will be dark gray. |
| stimulate a left eye ON | User's percept will be gray | |
| hypercolumn quadrant | (no stimulus). | |
| and energizing emitter | | |
| B to stimulate a left eye | | |
| OFF hypercolumn quadrant. | | |

The left eye response signal information classification chart of Table B can be repeated for the right eye of user 20. In a first iteration of emit block 1202 according to one embodiment, implant system 100 can select for control for each hypercolumn 12 of cortical column array 8 represented by cortical map 10 the emitters at positions p and x (FIG. 1E) for a first pair of A and B emitters targeted for a first pair of left ON and OFF hypercolumn quadrants of a respective hypercolumn 12, and the emitters at positions r and z (FIG. 1E) for second pair of A and B emitters targeted for a second pair of right ON and OFF hypercolumn quadrants of the respective hypercolumn 12. The selected pairs of emitters can be selected to have a pitch (spacing) coordinated with and according to a pitch (spacing) of hypercolumn quadrants of a user, and since the emitter array 140 of FIG. 1E can have a pitch of ¼× the hypercolumn quadrant pitch of cortical column array 8 represented by cortical map 10, selected emitter pairs for emission control at emit block 1202 can include emitters at spaced apart emitter positions.

Emitters selected for control at an initial iteration of emit block 1202 can include less than all emitter of emitter array 140 so as to reduce a likelihood of adjacent emitters simultaneously stimulating a common hypercolumn quadrant. While emitters can be selected to reduce the likelihood of simultaneous stimulation by adjacent emitters, embodiments herein recognize that the scale of cortical column array 8 represented by cortical map 10 can facilitate alignment of a single emitter of emitter array 140 to a certain one hypercolumn quadrant.

Embodiments herein recognize that pixel positions provided by hypercolumns of cortical column array 8 represented by cortical map 10 can be tens to hundreds of times larger than pixel positions of analogous electronic equipment. For example, whereas digital image sensors can have pixel sizes smaller than 1 micron, an adult human can have in one example aligned emitters for the available hypercolumn quadrants when using emitters of about 200 microns in size (more than 200 times larger in terms of pitch in one dimension, more than 40,000 times larger than a camera CCD or CMOS pixel in terms of pixels per unit area), according to one example. Embodiments herein recognize that the noted size differential and scale of cortical map 10 facilitates straightforward alignment processes for discovery of aligned emitters to the hypercolumn quadrants and therefore targeted stimulation and excitation of hypercolumn quadrant targets.

FIG. 1G illustrates emitter array 140 of implant system 100 interoperating with cortical column array 8 represented by cortical map 10. In use, emitters of emitter array 140 can transmit emission light a distance from its implantation location at the surface of a visual cortex to a depth within cortex of about 1 mm—to stimulate layer 4 of a user's cortical column array 8 represented by cortical map 10, where LGN cellular projections 30 (made light sensitive by optogenetics) interface to neurons in layer 4 of the user's primary visual cortex. Whereas brain tissue can scatter light from the depicted emitter so that the emitter can diffuse a slightly diverging cone of light around the radiated beam, the large pitch (e.g., about 500 microns according to one example) of hypercolumn quadrants facilitates alignment of an emitter to individual hypercolumn quadrants, despite diffusion from light scatter of the emitter beam. Implant system 100 can be configured so that when an emitter of emitter array 140 emits light, a particular hypercolumn quadrant can be stimulated. In one aspect, implant system 100 can be configured so that when an emitter of emitter array 140 emits light, cellular projections of LGN 30 (made light sensitive by optogenetics) in a localized area about a particular area interfaced to a hypercolumn quadrant can be activated to stimulate and excite the particular hypercolumn quadrant of a hypercolumn 12, causing the particular hypercolumns quadrant to luminesce. In some embodiments, for aiding alignment of emitters to hypercolumn quadrants, emitters of emitter array 140 can have features to restrict a divergence angle by which light emissions diverge from the emitters.

In one example of emit block 1202 implant system 100 can present a single calibration emission frame to cortical column array 8 represented by cortical map 10, e.g., a single frame to evoke the perception of “light” or alternatively “dark” or alternatively “gray” at particular hypercolumns 12 of cortical map (different hypercolumns can simultaneously be presented with different combinations of “light” evoking emissions, “dark” evoking emissions, and “gray” evoking emissions). In other examples, implant system 100 at emit block 1202 can present a sequence of calibration frames to cortical column array 8 represented by cortical map 10, in which case detection block 1203 can include multiple detection stages in which response signals associated to each emission frame can be read out for obtaining response signal information associated to a sequence of calibration emission frames.

At detection block 1203, implant system 100 can detect response signals in response to emission signals sent at emit block 1202 with use of detector array 150. Detectors of detector array 150 can receive bioluminescently or fluorescently emitted photons from neurons in the primary visual cortex that have been modified transgenically to produce light-emitting or fluorescent proteins that vary in their emission as a function of activity level (due to changes of calcium and/or voltage in the neurons of the primary visual cortex). These proteins will be multicolored, and detector array 150 will be a hyperspectral array of spectrophotometers, allowing the readout system to sample the response of small groups neurons and even single neurons. Single unit recordings transmitted to local system 200 and remote system 400 can be stored and/or reconstructed to show what user 20. Detector array 150 can have a plurality of detectors (indicated as rectangles in FIG. 1E) that can be interleaved with emitters of emitter array 140. Detector array 150 and emitter array 140 can be configured so that there is one detector associated to one emitter in an emitter detector dyad as set forth herein. Embodiments herein recognize that a given detector can receive bioluminescent signals from the brain that are derive from its associated emitter in its dyad. While a particular emitter can be associated to a particular detector in its dyad, embodiments herein also recognize when multiple emitters are firing in patterns it will excite new neurons that respond to patterns of inputs because they are connected with distant parts of cortex through lateral connections in the brain circuits (such as neurons that are tuned to oriented edges of objects in the visual scene). As such, an emitter at one cortical retinotopic position may be influenced by distant emitters, but that this will not significantly degrade the signal, which is based on spatiochromatic signals.

Embodiments herein recognize with reference to FIG. 1G that if a given hypercolumn quadrant 11 is illuminated so as to activate LGN cells terminating in hypercolumns 12 of FIG. 1G, (including, e.g. LGN boutons in Layer IV), the cortical neurons within the targeted hypercolumn quadrant, in the volumetric area within and immediately above and below Layer 4 can be stimulated to excite and luminesce in a localized volumetric area which luminescence can be detected by the detector of detector array 150 associated to a particular emitter of emitter array 140.

Referring again to the flowchart of FIG. 1F, implant system 100 at block 1205 can classify emitters controlled at emit block 1202 with use of a call chart as set forth in reference to Table B (replicated for a right eye), as being aligned, misaligned, or insufficient information, and at block 1206 implant system 100 can record the classification. Determining that an emitter is not aligned can result from failing to satisfy a target characteristic of alignment or satisfying of a target characteristic of misalignment. At block 1207 implant system 100 can ascertain whether a current emitter classified is a last emitter subject to control at prior emit block 1202 and can iterate the loop of blocks 1204-1207 until a last emitter subject to control at the most recent iteration of emit block 1202 has been classified. When a last emitter subject to control at a most recent iteration of emit block 1202 has been classified, implant system 100 can proceed to block 1208 to ascertain whether all emitters of emitter array 140 have been classified as aligned or misaligned, and if there remain emitters not classified as aligned or misaligned, implant system 110 can proceed to block 1209 to update emission parameter values for use in a next iteration of emit block 1202, and then can return to emit block 1202 to perform a next iteration of emit block 1202 in which selected emitters of emitter array can be controlled to be energized or deenergized at a selected power delivery level (controlled with use of, e.g., emission amplitude control and/or pulse width modulation).

At block 1209 implant system 100 can update emission parameter values for performance of a next iteration of emit block 1202 based on record data recorded at block 1206 For example, where a prior emitter has been classified as being aligned or misaligned, that emitter can removed from a candidate list of emitters for control in a next iteration of emit block 1202. At a next iteration of emit block 1202 implant system 100 can subject to control emitters that have not previously been subject to control in a prior iteration of emit block 1202 and/or emitters that have been classified with the described insufficient information tag.

It will be seen with reference to FIG. 1E that if emitters at positions p, x, r, and z, for example, are subject to control to ascertain alignment with hypercolumn quadrants 11 of a certain hypercolumn 12 at a first iteration of emit block 1202 and alignment is not discovered, the emitters at the alternative positions, e.g., o, w, q, y (FIG. 1E) might be subject to control at a next iteration of emit block 1202 for discovery of emitters aligned to hypercolumn quadrants 11 of the certain hypercolumn 12 and emit block 1202 can continue to be iterated in performance of the loop of blocks 1202-1209 until at block 1208 implant system 100 determines that each emitter of emitter array 140 has been classified as being aligned or misaligned.

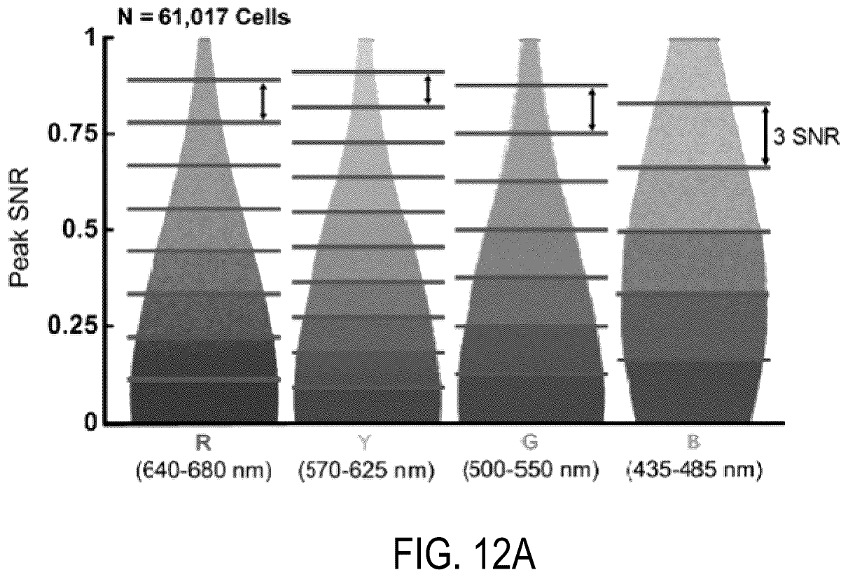

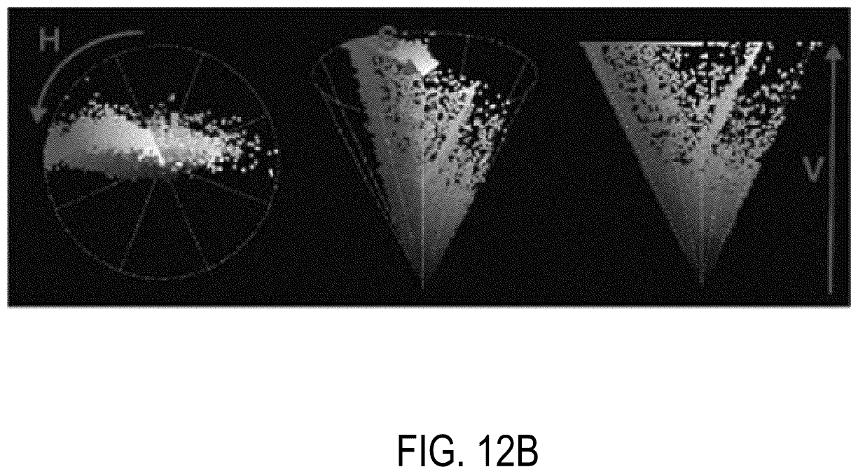

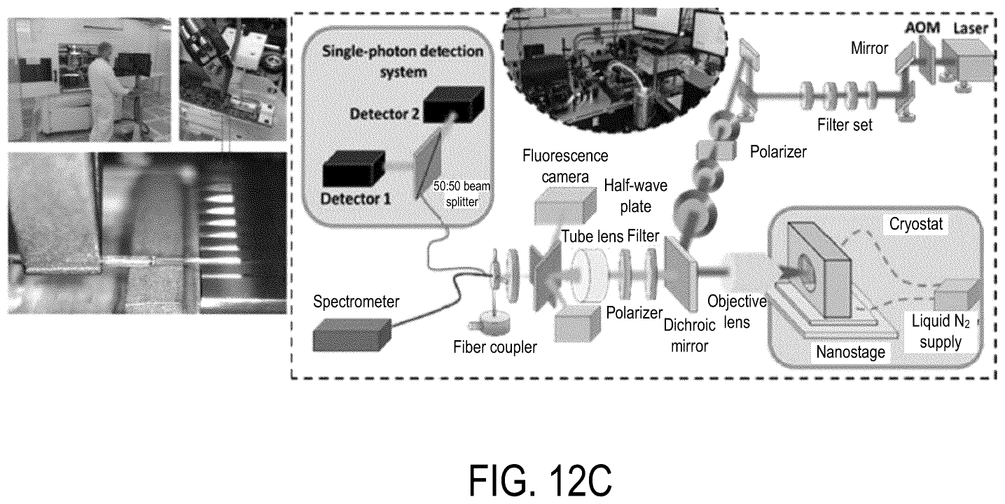

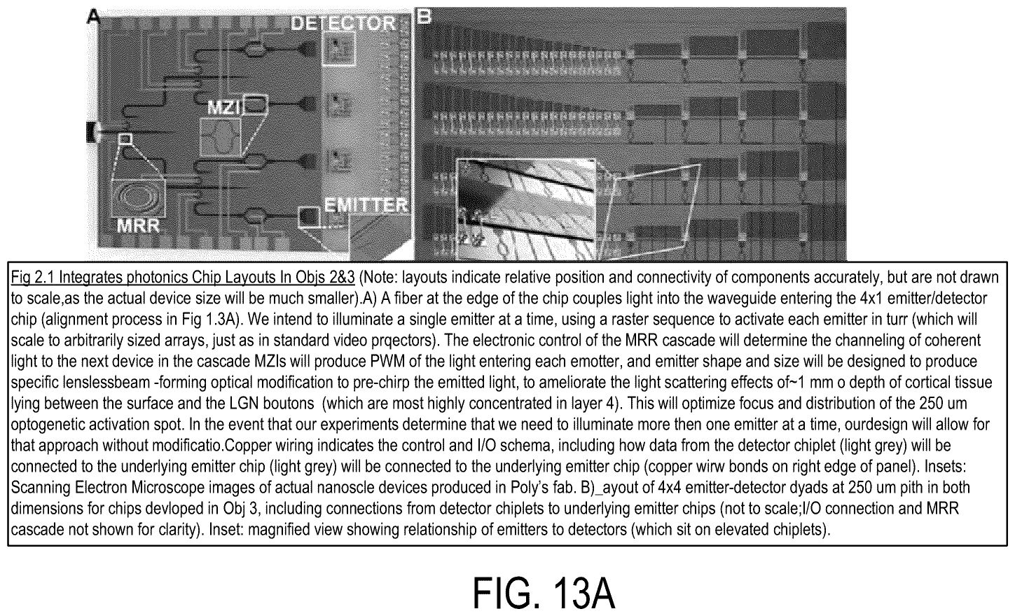

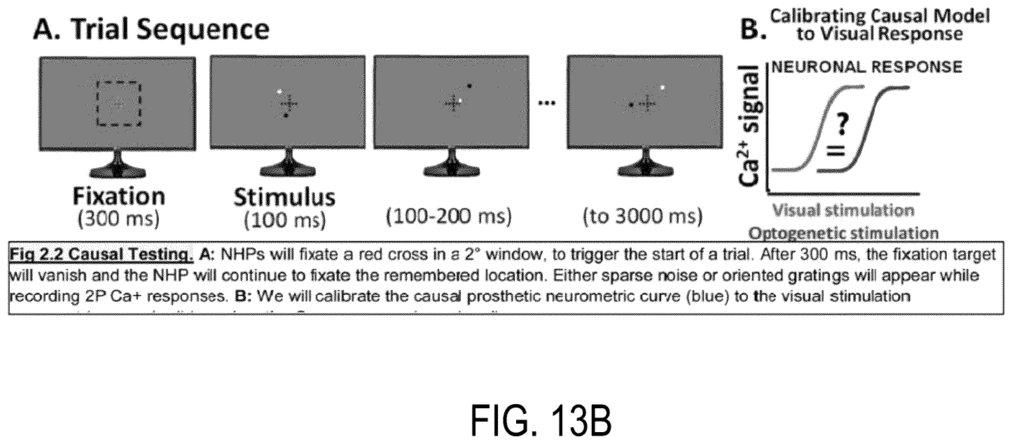

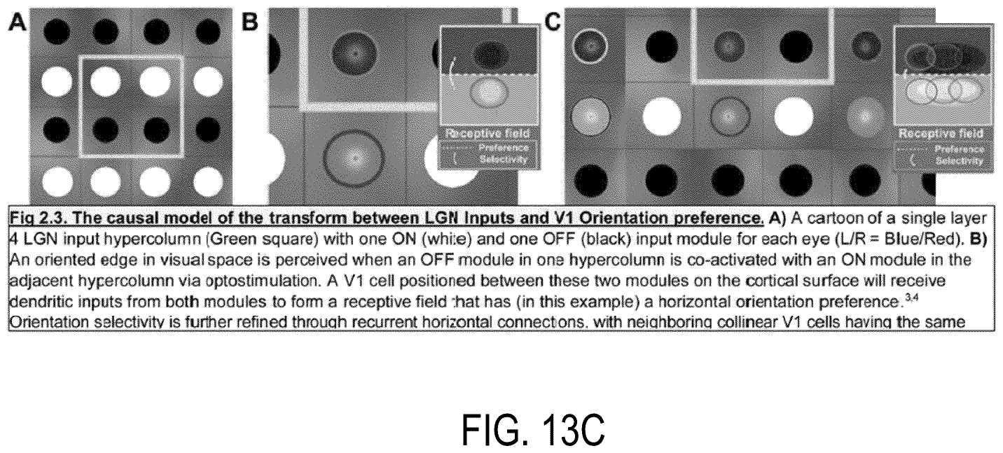

Embodiments herein recognize that where multiple emitters throughout regions of emitter array 140 are subjection to emission simultaneously, alignment or misalignment of each or substantially each emitter of emitter array 140 with respect to a hypercolumn quadrant of cortical column array 8 represented by cortical map 10 can be discovered within a limited number of iterations of emit block 1202. In one aspect, white noise processing can be performed to ascertain alignment or misalignment of emitters of emitter array 140 with respect to a hypercolumn quadrant of cortical map. At iterations of record block 1206, implant system 100 can record the information of the identified aligned emitters within data repository 1080. On the determination at block 1209 that a candidate pair of emitters that has been tested is not aligned, implant system 100 can return to a block 1201 in a next iteration can try a second candidate pair of emitters. The described iterative re-trying can occur rapidly in real-time at video rates (e.g., from about 24 Hz to about 1000 Hz) to optimize the emitters that are used, their power delivery (e.g., by amplitude and/or pulse-width control) on each stimulation frame presented by emitter array 140 to cortical column array 8 represented by cortical map 10 at iterations of emit block 1202. Implant system 100 can iteratively perform emit block 1202 (in a raster pattern in the array, or in another sequential pattern) in performing iterations of the loop of block 1202 to block 1209 until a set of candidate emitters have been identified as being aligned emitters aligned to hypercolumn quadrants of cortical column array 8 represented by cortical map 10. In one embodiment, this process can also be done rapidly and efficiently by testing the emitter/detector pairing to hypercolumn quadrants using m-sequenced white noise (which will test half of the emitter/detector dyads simultaneously in a known but pseudo-random pattern that varies at video rate, which allows for faster calibration than sequential rasterized scanning through the array). FIG. 13A depict chip layouts of the present disclosure, according to one example. (Note: layouts indicate relative position and connectivity of components accurately, but are not drawn to scale, as the actual device size will be much smaller). FIG. 13A (top lefts photograph) depicts a fiber at the left edge of the chip couples light into the waveguide entering the 4×1 emitter/detector chip (alignment process in FIG. 12C). It is intended to illuminate a single emitter at a time, using a raster sequence to activate each emitter in turn (which will scale to arbitrarily sized arrays, just as in standard video projectors). The electronic control of the MRR cascade will determine the channeling of coherent light to the next device in the cascade. MZIs will produce PWM of the light entering each emitter, and emitter shape and size will be designed to produce specific lensless beam-forming optical modifications to pre-chirp the emitted light, to ameliorate the light scattering effects of ˜1 mm of depth of cortical tissue lying between the surface and the LGN boutons (which are most highly concentrated in layer 4). This will optimize focus and distribution of the 250 um optogenetic activation spot. In the event that our experiments determine that we need to illuminate more than one emitter at a time, our design will allow for that approach without modification. Copper wiring indicates the control and I/O schema, including how data from the detector chiplet (light grey) will be connected to the underlying emitter chip (copper wire bonds on right edge of panel). Insets: Scanning Electron Microscope images of actual nanoscale devices produced in fabs.⁸ FIG. 13A (top right) depicts a layout of 4×4 emitter-detector dyads at 250 um pitch in both dimensions for chips of the present disclosure, including connections from detector chiplets to underlying emitter chips (not to scale; I/O connections and MRR cascade not shown for clarity).

On the determination at block 1208 that each emitter of emitter array 140 has been classified as being aligned or misaligned, implant system 100 can proceed to block 1210 to register a calibration map for emitter array 140 in data repository 1080. The calibration map can specify the classification (aligned or misaligned) for each emitter of emitter array 140. Implant system 100 for ensuing use of emitter array 140 can disable emitters classified in the calibration map emitters classified as being misaligned and can enable emitters of emitter array 140 that are classified in the calibration map as being aligned. By being disabled, an emitter is restricted from being controlled to emit light. By being enabled, an emitter can be capable of being controlled to emit light.

Embodiments herein recognize that different hypercolumn quadrants 11 of cortical column array 8 represented by cortical map 10 can have can radiate differentiated colors upon being stimulated and excited to luminesce. According to one embodiment, the color radiated by respective hypercolumn quadrants on stimulation and excitation of cortical column array 8 represented by cortical map 10 can be detected during the calibration loop of blocks during the calibration loop of blocks 1202-1208 and recorded at block 1206 so that a color signature of detected response signals at pixel positions defining cortical column array 8 represented by cortical map 10 is recorded as part of the calibration map registered at block 1210. In some use cases, emitters can be controlled on a one emitter at a time basis for avoiding any cross talk during the color signature identification and recording process. Embodiments herein recognize that at detect block 1203 and detect block 1214 detected response signals detected with detector array 150 and associated to an emission signal can be substantially localized so that luminescence detected as result of an emission by a certain emitter can be detected with use of a detector associated to the emitter. In another aspect, to the extent there may be crosstalk between emitters and detectors, system 1000 using the color signature data of the registered calibration map can for detection of response signal associated to a certain emitter filter out response signal not attributable to excitations resulting from emissions by the certain emitter. For example, detectors of detector array 150 can include associated tunable filtration devices as explained with reference to FIGS. 15A-15D and implant system 100 can be configured to set the various tunable filtration devices to pass wavelengths in dependence on the registered color signatures of the described calibration map, in order to reduce cross-talk. Accordingly, system 1000 can be configured to identify a source location of a response signal based on a determined color of the response signal, wherein the response signal is detected with use of the detector of the plurality of detectors.

Embodiments herein recognize with reference to Table B that, in some instances, the actual response data will not map perfectly to the nominal targeted response data or the nominal misalignment indicating response data. In such situations, implant system 100 can apply, e.g., clustering analysis to select the best fit classification, e.g., aligned or not aligned, for each tested pair of emitters tested with emission signal emissions at emit block 1202. Further, embodiments herein recognize that due to contributing factors such as cross talk between hypercolumns, a percentage of emitters determined to be aligned to a hypercolumn quadrant may actually be misaligned. Embodiments herein recognize that even with a percentage of emitters being misaligned that are recorded in a calibration map as being aligned to a hypercolumn quadrant, image information that is precisely emitted to cortical column array 8 represented by cortical map 10 via aligned emitters can be sufficient for the delivery of discernible and useful artificially stimulated image information to user 20.

On completion of block 1209, implant system 100 can proceed to block 1210. At block 1210 implant system 100 can send a ready signal data to artificial sensory system 300 to signal to artificial sensory system 300 that implant system 100 is ready to receive live streaming video data. On completion of block 1209, there can be stored within data repository 1080 calibration data that specifies which emitters of emitter array 140 are aligned to particular hypercolumn quadrants 11 defining cortical column array 8 represented by cortical map 10.

With the calibration data complete, implant system 100 has information of which emitters of emitter array 140 are to be enabled (capable of emissions in which emitters are disabled) incapable of emissions during an ensuing artificial viewing session in which a user can be presented image data, e.g., streaming image data.

In response to receipt of the ready signal data sent at block 1210, artificial sensory system 300 at block 3201 can send streaming video scene data obtained using scene camera image sensor 160 and streaming video eye movement image data obtained using eye tracking camera image sensor 170 for receipt by local system 200 which local system 200 then can be configured to redirect the streaming data to implant system 100. Note that only the subregion of the scene camera's image on the retina that corresponds to the cortical region that is stimulated may be sent from the spectacles to the brain. Such tracking can be achieved by tracking the eye position's gaze within the scene in real-time.

In response to the streaming eye movement image data, local system 200 at recognizing block 2205 can perform recognizing of spatial information image data represented information of an eye of a user 20. Local system 200 running an image recognition process can examine spatial image data representing an eye of user. Local system 200 running an image recognition process can include local system 200 employing pattern recognition processing using one or more of, e.g., feature extraction algorithms, classification algorithms, and/or clustering algorithms. In one embodiment, local system 200 running image recognition process can include local system 200 performing of digital image processing. Digital image processing can include, e.g., filtering, edge detection, shape classification, and/or encoded information decoding. This process will ensure that the stimulation of the brain by the emitter array 140 replicates the image that the natural visual system would send to primary visual cortex, including any and all subcortical image processing that might occur in the retina and lateral geniculate nucleus before the information is sent to the primary visual cortex (V1) of user 20.

The recognizing performed at block 2205 can include recognizing to ascertain a current horizontal and vertical position of an eye of user 20. Various classifications of eye position of a user can be ascertained at block 2205. In one embodiment, with use of image recognition processing, an eye position of user 20 in terms of horizontal and vertical position can be resolved with accuracy of within less than about 2 degrees and in one embodiment to an accuracy of within about less than 0.5 degrees.

In one embodiment, frame image data streamed to a user 20 by emissions of emitter array 140 to a cortical column array 8 represented by cortical map 10 can include controlled emissions mapping to a subset of pixel locations of a frame of image data produced using scene camera image sensor 160. In one embodiment, system 1000 can be configured so that the subset of pixel locations of the frame of image data produced using scene camera image sensor 160 mapping to controlled emissions of emitter array 140 can change in dependence on a current eye position of user 20. In one embodiment, system 1000 can select a subset of pixel positions controlling emissions by emitter array 140 in dependence on a detected current eye position of a user. In one embodiment, a subset of pixel positions comprising a center set of pixel positions can be selected in the case the user is detected to have a central gaze eye position. System 1000 can be configured so that in the case the user is detected to have a horizontal and/or vertical eye position shifted from a central gaze position the selected set of pixels positions controlling emissions by emitter array 140 can be shifted accordingly. In one embodiment, image data of subset of pixel positions defining the scene camera's field of view can be transmitted to the brain via the emitter array 140 and the position of that subset will be determined by the horizontal and vertical eye position of user 20 in each eye individually. The eye position of user 20 can be iteratively determined to iteratively adjust the selected subset of pixel positions.

At block 2206, the described pixel position selection can be performed in response to the detected eye position detected at recognizing block 2205. In response to completion of block 2206 local system 200 can proceed to block 2207. At block 2207 local system 200 can send streaming image data selectively associated to the selected truncated pixel positions for receipt by implant system 100.

On completion of block 2207 local system 200 can proceed to block 2208. At block 2208 local system 200 can determine whether a current live artificially stimulated artificial viewing session has been terminated e.g., by user input control into a user interface of local system 200. In response to determination that an artificial viewing session has not been terminated, local system 200 can return to a stage preceding block 2205 in order to perform a next iteration of recognizing at block 2205 by processing streaming video eye movement image data produced using eye tracking camera image sensor 170 having a field of view, a, encompassing an eye have a user 2. Local system 200 can iteratively perform the loop of blocks 2205 to 2208 for a duration of an artificial viewing session and in the iterative performing of recognizing at block 2205 and selecting block 2206, can iteratively adjust selected pixel positions for transmission for transmission to a user in dependence on the detected current eye position of user 20 detected at block 2205.

In response to receipt of streaming image data sent at block 1207 implant system 100 at select block 1212, can select a power delivery level for transmitted emission light transmitted to a user by control of selected emitter of emitter array 140. Streaming video data sent at block 2207 to implant system 100 can include streaming video scene image data defined by truncated frames of image data truncated with use of eye position selection described.

At the first iteration of select block 1212 for selecting a power delivery level emitter power delivery level can be set to a nominal predetermined power delivery level based on historical data, e.g., based on historical data of multiple users of system determined to return usable response signal data. In response to power delivery level selection block 1212, implant system 100 can proceed to emit block 1213. At emit block 1213, implant system 100 can transmit streaming video data to cortical column array 8 represented by cortical map 10 in order to stimulate and excite select hypercolumns of cortical column array 8 represented by cortical map 10. At emit block 1213, implant system 110 can use the described calibration map data stored in data repository 1080 so that only select emitters of emitter array 140 determined to be aligned to particular hypercolumns of cortical column array 8 represented by cortical map 10 are enabled and further so that select emitters of emitter array 140 are disabled and are not controlled to emit light during a live artificially stimulated artificial viewing session in which moving frame image data can be emitted to a cortical column array 8 represented by cortical map 10 of user 20 with use of emitter array 140. The nominal targeted response signal can be based on a balance of factors. In one embodiment, the targeted response signal can have targeted characteristics, e.g., in terms of response signal amplitude based on experimental data associated to multiple users that is indicative of a response signal amplitude that generates well-functioning scene reproduction without risk of brain tissue damage to user.

In one aspect, implant system 100 can be configured to present to user's cortical column array 8 represented by cortical map 10 by light emissions using emitter array 140 image data defining a scene in response to and in dependence on frame image data sent by local system at block 2207, wherein frame image data can be presented in a sequence of frames defining moving frame streaming image data. For presenting by emissions frame image data by implant system 110 to cortical column array 8 represented by cortical map 10, implant system 110 can present, e.g., dark space information, light space information or gray space information to particular ones of cortical pixels defining cortical column array 8 represented by cortical map 10 in the manner described herein. Namely, in order to present light space information an emitter for activating an ON hypercolumn quadrant can be energized and the emitter for activating an OFF hypercolumn quadrant can be deenergized. For presenting dark space information, implant system 100 can control an emitter for an ON hypercolumn quadrant to be deenergized and can control the emitter for an associated OFF hypercolumn quadrant to be energized. For presenting gray space information to a user, implant system 100 can control an emitter for both ON and OFF hypercolumn quadrants to be energized, and the appearance of the gray level can be determined by the ratio, relative emission power amplitude, and relative pulse-width modulation of the ON versus OFF hypercolumn quadrants.

Implant system 100 can be configured, e.g., with use of table lookup, to transform color or grayscale streaming image data received from local system 200 into the pixelated image data comprising pixel positions having the described values of light space, dark space and gray space.

Further, pixel value aggregating or interpolation techniques for transformation of pixel resolution of incoming scene representing image data into a pixel resolution associated to emitter array 140, which pixel resolution can be selected to map to a pixel resolution defined by cortical column array 8 represented by cortical map 10. Referring to FIG. 1E, emitter array 140 can be selected to have a pixel resolution mapping to a pixel resolution of cortical column array 8 represented by cortical map 10 and emitters at respective pixel positions of emitter array 140 can be controlled to stimulate and excite hypercolumn quadrants at corresponding pixel positions of cortical column array 8 represented by cortical map 10. For example, emitters at pixel position A1 of emitter array 140 can be controlled to stimulate and excite hypercolumn quadrants of corresponding pixel position A1 of cortical map, emitters at pixel position B1 of emitter array 140 can be controlled to stimulate and excite hypercolumn quadrants of corresponding pixel position B1 of cortical map, and so on. As set forth herein, each pixel position of emitter array 140 can be configured to have more emitters than are needed to stimulate the number of hypercolumns at the corresponding pixel position of cortical map. That configuration, as explained herein, is to increase the likelihood of there being alignment of an emitter to respective hypercolumn quadrants of cortinal map, which alignments can be discovered by a calibration process set forth herein. For stimulation and excitation of a hypercolumn quadrant, an emitter can activate an input to hypercolumn quadrant.

Implant system 100 can present frames of image data to cortical column array 8 represented by cortical map 10 in dependence on frames of image data received by implant system 100 wherein the received frames have been obtained using the scene camera image sensor 160. The frames of image data sent to cortical column array 8 represented by cortical map 10 with use of emitter array 140 and the frames of image data received by implant system 100 can be pixelated moving frames of image data, wherein different pixel positions have associated pixel values. In one embodiment, implant system 100 can perform various operations in presenting a frame of image data to cortical column array 8 represented by cortical map 10 in dependence on received image data frames of image data. Such operations can include, e.g., changing resolution of a received frame of image data, if needed to match pixel resolution of emitter array 140, converting color image data of a received frame of image data into a gray scale in the case gray scale image data is to be presented to cortical column array 8 represented by cortical map 10, converting color image data of a received frame of image data into binary image data if binary image data is to be presented to cortical column array 8 represented by cortical map 10, and determining emitter controls for emitters at the pixel positions of emitter array 140 so that frame image data is emitted to cortical column array 8 represented by cortical map 10 according to the frame image data received by implant system 100. Emitters of emitter array 140 at particular pixel positions of emitter array 140 can be controlled so that hypercolumn quadrants 11 can be appropriately stimulated to produce perceptions of light, dark, and gray accurately according to scene representing frame image data received by implant system 100. As noted herein, the particular emitters controlled to present frame image data to cortical column array 8 represented by cortical map 10 of user 20 for a certain pixel position can include only a subset of emitters within the certain pixel position of emitter array 140 discovered during calibration processing to be aligned with a hypercolumn quadrant defining cortical column array 8 represented by cortical map 10. In some embodiments, binary image data can be presented to a user's cortical column array 8 represented by cortical map 10. In some use cases, gray scale image data can be presented to a user's cortical column array 8 represented by cortical map 10. For presenting binary image data to a user's cortical column array 8 represented by cortical map 10, pixel values associated to pixel positions of an incoming scene representing frame of image data received by implant system 100 can be converted to the binary variable that can assume the value 0=dark or 1=light.

In response to the emission at emit block 1213 cortical column array 8 represented by cortical map 10 at block 1103 can send a response signal, which can be detected by implant system 100 in response to receipt of the response signal. Blocks 1214-1216 can then be performed. At block 1217 implant system 100 can determine whether a current artificial viewing session has been ended, e.g., by actuation of a control of local system 200 by user 20. For the time that a current artificial viewing session has not been terminated, implant system 100 can iteratively perform the loop of blocks 1212-1217. At first, second and subsequent iterations of select block 1212, implant system 100 can set a power delivery level associated to each new frame presented to cortical column array 8 represented by cortical map 10 of the user with use of emissions by emitter array 140. Power delivery can be controlled with user of emission amplitude control and/or emission on time control (e.g., pulse width modulation). Implant system 100 at emit block 1213 can send via light emission to cortical column array 8 represented by cortical map 10 frames of streamed frames defining a stream of image data on a frame by frame basis. For example, in a first iteration of emit block 1213, implant system 110 can send via light emissions a first frame of image data defining a sequence of frames to cortical column array 8 represented by cortical map 10 and at a next iteration of emit block 1213, implant system 100 can send via light emissions a subsequent frame of image data defining a succession of frames, wherein the succession of frames defines streaming image data. Emissions by emitter array 140 at block 1201 and emit block 1213 can be regarded to be light field emissions.

Implant system 100 can be configured so that at select block 1212, implant system 100 sets a power delivery level for a subsequent frame to be emitted at emit block 1213 in dependence on detected response signal during a prior iteration of response signal detection. As set forth herein, a power delivery level can be set with use of emission amplitude control and/or with use of emission on time control (e.g., pulse width modulation). In one embodiment, implant system 100 can detect whether a response signal has a targeted amplitude. Embodiments herein recognize that a number of factors can contribute to an amplitude associated to a response signal sent by cortical column array 8 represented by cortical map 10 at block 1103. The response signal amplitude can be dependent, e.g., not only on the emission power delivery level but on other factors, e.g., physiological characteristics of the current user to the state of the user. Many factors are known to affect response signals in cortex, such as attention level, levels of consciousness, sleep state, the presence of caffeine or other pharmaceuticals, changes in the light level of the ambient visual environment, etc.

In one embodiment, implant system 100 at select block 1212 can adjust a power delivery level associated to an emitter upward or downward depending on a characteristic, e.g., amplitude of a detected receipt signal received at a last iteration of detection so that over time, a targeted characteristic of returned response signal returned by a hypercolumn quadrant, e.g., response signal amplitude, can stay regulated proximate at targeted characteristic. In some embodiments, different users can have different targeted response characteristics. The response signal can be read out through the detector array 150, which can receive bioluminescently or fluorescently emitted photons from neurons in the neocortex, e.g., primary visual cortex (V1) of user 20 that have been modified transgenically to produce light-emitting or fluorescent proteins that vary in their emission as a function of activity level (due to changes of calcium and/or voltage in the neurons of the neocortex, e.g., primary visual cortex). These proteins can be multicolored, and detector array 150 can be provided by a hyperspectral array of spectrophotometers, allowing the readout system to sample the response of small groups neurons and even single neurons. To prevent undesirable activation of ontogenetically active thalamic inputs by bioluminescent/fluorescent photons from neocortex, Bioluminescent or fluorescent colors can be engineered, i.e., selected, to emit in a wavelength band that does not overlap with the sensitive wavelength band of the channelrhodopsins utilized in making thalamic inputs to neocortex sensitive to light. Single neuron recordings transmitted to local system 200 and remote system 400 can be stored and/or reconstructed to show what user 20 viewed in the world.

In one embodiment, emission power delivery levels associated to different emitters of emitter array 140, can be controlled differently. For example, in one embodiment, implant system 100 can be configured so that ON state power delivery levels associated to each respective emitter of emitter array 140, can be set independently to a selected power delivery level controlled by controlling emission amplitude and/or by control of emission on time (pulse-width duration) selected at select block 1212. Configuring implant system 100 to independently control power delivery levels to respective emitters of emitter array 140 can provide precise control of light energy transmitted to a user's primary visual cortex, thereby providing contrast control with up to 10 bits of depth, as well as to limit potential risk of brain tissue damage to the user. Given that the loop of blocks 1212-1217 iterates, embodiment herein recognize that implant system 100 can iteratively adjust the different selected power delivery for each enabled emitter of emitter array 140 over time.

Embodiments herein recognize that different areas of a primary visual cortex (V1) can exhibit different response characteristics. For example, a first section of a V1 can be highly responsive and produce a large response signal in response to a baseline emission signal at a baseline power delivery level, whereas a second section of a V1 can provide a small response signal in response to a baseline emission signal sometimes reducing the quality of scene reproduction. In such a scenario, system 1000 at a second iteration of select block 1212, based on a detected response signal can increase power delivery level of emissions by a first emitter of emitter array 140 to the first section and can decrease power delivery level of emissions by a second emitter of emitter array 140 to the second section so that an amplitude of a returned response signals from the first and second sections of a V1 can be substantially normalized and made substantially equal.

The precise control of power delivery levels associated to different emitters across emitter array 140 can facilitate precise control of stimulation of hypercolumns defining cortical column array 8 represented by cortical map 10 increasing the accuracy with which scene image data can be represented while minimizing risk of damage to brain tissue defining cortical column array 8 represented by cortical map 10. In some use cases in which emission power level for emitters throughout emitter array 140 are determined to avoid response signal limits indicative of risk to user 20, the limits can be determined using maximally bright emitters associated to maximally light (brightest) pixel positions of emitter array 140, and power levels for remaining pixel positions of emitter array 140 can be scaled from the brightest pixel positions so that gray scale image data accurately representing the scene representing image data received by implant system 100 is presented by emitter array 140 to cortical column array 8 represented by cortical map 10 by way of photonic emissions.

In one aspect, implant system 100 can include readout circuitry for reading out frames of image data from detector array 150. At detect block 1214, based on response signal information transmitted at block 802 implant system 114, with use of the described readout circuitry can be reading out frames of image data that can include image data associated to respective pixel positions of detector array 150. Detector array 150 can have pixel positions such as pixel positions A1 through G7 as described in FIG. 1E. At processing block 1215, implant system 100 can be performing processing for power regulation as described in connection with select block 1212.

At processing block 1215, in one embodiment, implant system 100 can be checking every single stimulation pulse emitted by emitter array 140 and its return to assess the quality of calibration, and if patterns are observed that indicate that implant system 100 should be recalibrated (for example the implant may be shifting laterally with respect to the cortex due to a head impact) implant system 100 can perform recalibration at block 1215 to update the calibration map registered at block 1210 to enable/disable emitters in real-time as part of the current artificial sensory (e.g. streamlining video input) session, or can trigger a return to the calibration loop at blocks 1208-1210 inclusive of a command to return to the current sensory input session of the loop of blocks 1212-1217 when recalibration is complete.

At processing block 1215, implant system 100 herein can also or alternatively be performing processing for reconstruction of a visual scene that has been viewed by user 20. Such processing for reconstruction of a visual scene can include, e.g., transforming response signal associated to various hypercolumn quadrants into scene information that has been perceived by the user. For example, a detected luminescence by a detector associated to an OFF hypercolumn quadrant can be converted into a pixel value indicating dark space information. Implant system 100 can be using mapping transformation data structures in order to convert hyper column quadrant detector output data into interpolated perceived pixel values representative of scene image data that has been perceived by the user.

In another embodiment for preparing a frame of read-out image data for transmission at process block 1215, implant system 110 can simply record digitized representations of the raw detector output values output by detectors associated with various pixel positions of detector array 150. Conversion processing, if performed at all can be performed by any computing node in the transmission path. In another embodiment, implant system 100 at process block 1215 for processing a prepared frame for transmission can provide a prepared frame with both converted grayscale pixel position image data indicating interpolated scene information that has been viewed by user 20 as well is raw frames of image data. The raw frame read-out option can be advantageous in a wide range of scenarios including scenarios in which the artificial sensor input that is emitted to cortical column array 8 with emitter array 140 is other than visual. At send block 1216, implant system 100 can be sending and transmitting, i.e., on a streaming video basis read-out frames of image data to local system 200. The transmitted frames of read-out image data can be formatted in raw format, wherein the raw signal data may be merely subject to digitation, or in processed format characterized by more advanced processing. Local system 200, in turn, can relay the read-out frames at send block 2209 to remote system 400, which in turn can relay the read-out frames to remote computing environment, e.g., remote computing environment 1100A of computing environments 1100A-1100Z, which in turn can process the read-out frames.

Processing at block 1102 by a remote computing environment, e.g., computing environment 1100A can include processing the read-out frames to facilitate administrator user review at the location of the remote computing environment to review and analyze at the remote location, e.g., by display on a display of a computing node at the remote location. The administrator user at the remote location of computing environment 1100A can observe whether user 20 has been properly stimulated with image data. Processing at block 1102, at block 4204 by remote system 400, at block 2210 by local system 200, and/or at block 1215 by implant system 100 can additionally, or alternatively, include, e.g., recognition processing to recognize features represented in read-out frames of image data as set forth herein, data logging processing, and/or machine learning processing. According to machine learning processing, iteration of image data of an input emitted frame of image data presented at emit block 1213 can be applied as training data to a predictive model together with iterations of image data of the read-out frame detected at block 1214 based on response signal information transmitted at block 802. Trained as described, system 1000 is able to learn attributes of a relationship between emitted input frame data and response frame data. System 1000 can thus query the described predictive model to ascertain a characteristic of an emitted frame of image data that can produce a targeted response, and can responsively transmit a frame of image data to implant system 100 having the characteristic, and in dependence of the frame, implant system can present emitted frame image data to cortical column array 8 represented by cortical map 10.

As set forth herein, scene representing image data sent to implant system 100 for controlling emissions by emitter array 140 can include scene representing image data obtained with use of a scene camera image sensor 160 that is separated from artificial sensory system 300 and in some cases remote from user 20. In one embodiment, a scene camera image sensor 160 can be disposed, e.g., on a manually or autonomously moving robot at a remote location remote from user 20, e.g., at remote computing environment 1100A or at a fixed point location remote from a user 20 at remote computing environment 1100A. The scene camera image sensor 160 can be disposed on an eyewear frame of a second user 20, which second user is located at the remote location remote from user 20, e.g., at computing environment 1100A (in this embodiment user 20 sees the field of view of the second user 20 who may be at a remote location). In another aspect, local system 200 at recognize block 2205 can be evaluating a data source for received streaming video data, i.e., can determine whether the received streaming video data subject to sending at block 2207 and possibly selecting at block 2206 is to be obtained from the local data source of artificial sensory system 300 worn by user 20 and specifically scene camera image sensor 160 of artificial sensory system 300 worn by user 20, or alternatively, whether the data source is a remote video data source such as remote system 400, which can be configure to stream and play back recorded video data or live video data, or whether the data source is a data source provided by a remote computing environment, such as computing environment 1100A of computing environments 1100A-1100Z. In some embodiments, remote system 400 can be configured so that remote system 400 at block 4202 relays obtained streaming video image data from a scene camera image sensor 160 disposed at a remote location of computing environment 1100A which can be iteratively streamed from computing environment 1100A at block 1101. The scene camera image sensor 160 can be disposed, e.g., on an eyewear frame worn by a second user 20 at that remote location and remote system 400 can be relaying the described streaming image data to local system 200 at block 4202, which can then relay the streaming image data to implant system 100 which can present emissions by emitter array 140 to cortical column array 8 to define presented frame image data in dependence on the described streamed frames relayed from remote computing environment 1100A. At block 2205, local system 200 can be examining control flags that can be set by any one of numerous users of system 1000 such as user 20 with use of a control, e.g., located on a handheld device of local system 200 or an administrator user associated with any computing node of system 1000.

On determination at block 1217, by implant system 100 that a current artificial viewing session has ended implant system 100 can proceed to return block 1218. At return block 1218 implant system 100 can return to a stage preceding block 1201 so that a next iteration of identifier data can be sent to local system 200. Implant system 100 can iteratively perform the loop of blocks 1201 to 1218 during a deployment period of implant system 100. Likewise, local system 200 at block 2208, can determine that a current artificial viewing session has ended, in which case local system 200 can proceed to return block 2211. Local system 2200 can in one embodiment can be configured to branch to perform block 2209 and block 2210 and return block 2211 while simultaneously performing the loop of block 2205-2205. At return block 2211, local system 200 can return to a stage preceding block 2201 so that a next iteration of identifier data from implant system 100 can be received. In some embodiments, the next iteration of identifier data can be associated to a different instance of implant system 100 associated to a different user 20. Local system 200 can iteratively perform the loop of blocks 2201 to 2211 during a deployment period of local system 200. At return block 803 cortical column array 8 represented by cortical map 10 can logically return to a stage preceding block 801 to be ready for next iteration of transmitting response signal information to implant system 100, under different stages of operation, e.g., calibration stage and live artificial viewing session stage, for example. Similarly, artificial sensory system 300 at return block 3202, can return turned to a stage preceding block 3201 to wait for next iteration of ready signal data being received from implant system 100. Computing environments 1100A-1100Z at return block 1103 can return to a stage preceding block 1101 and can iteratively perform the loop of blocks 1101 to block 1103.

In another aspect, artificial sensory system 300 can receive ready signal data from local system 200 based on the sending of ready signal data sent by local system 200 at block 2204 in response to determination at decision block 2201 that calibration is not to be performed, e.g., in the case the user is not a new user and/or the case that calibration has been performed within a threshold period of time. Remote system 400 at return block 4205 can return to a stage preceding block 4201 and can iteratively perform the loop of blocks 4201 to 4205 to iteratively send requested data to local system 200 during a deployment period of remote system 400. Remote system 400 can simultaneously be serving multiple instances of local system 200 throughout a wide area, e.g., countrywide, worldwide.

Processes described herein may be performed singly or collectively by one or more computer systems, such as one or more for executing secured program code. FIG. 1H depicts one example of such a computer system and associated devices to incorporate and/or use aspects described herein. A computer system may also be referred to herein as a data processing device/system, computing device/system/node, or simply a computer. The computer system may be based on one or more of various system architectures and/or instruction set architectures, such as those offered by Intel Corporation (Santa Clara, California, USA) or ARM Holdings plc (Cambridge, England, United Kingdom), as examples. FIG. 1H shows a computer system 500 which can be in communication with external device(s). Computer system 500 includes one or more processor(s) 110, for instance central processing unit(s) (CPUs). A processor can include functional components used in the execution of instructions, such as functional components to fetch program instructions from locations such as cache or main memory, decode program instructions, and execute program instructions, access memory for instruction execution, and write results of the executed instructions. A processor 100 can also include register(s) to be used by one or more of the functional components. Computer system 500 can also include a memory, input/output (I/O) devices 140, and communication I/O interfaces 180, which may be coupled to processor(s) 110 and each other via one or more buses and/or other connections. Bus connections defining a system bus 115 represent one or more of any of several types of bus structures, including a memory bus or memory controller, a peripheral bus, an accelerated graphics port, and a processor or local bus using any of a variety of bus architectures. By way of example, and not limitation, such architectures include the Industry Standard Architecture (ISA), the Micro Channel Architecture (MCA), the Enhanced ISA (EISA), the Video Electronics Standards Association (VESA) local bus, and the Peripheral Component Interconnect (PCI).

The memory can be or include working memory 120 provided by main or system memory (e.g., Random Access Memory) used in the execution of program instructions, and storage memory 130 as provided by storage device(s) such as hard drive(s), solid state non-volatile memory, flash media, or optical media as examples, and/or cache memory, as examples. Working memory 120 can include, for instance, a cache, such as a shared cache, which may be coupled to local caches (examples include L1 cache, L2 cache, etc.) of processor(s) 110. Additionally, the described memory comprising working memory 120 and storage memory 130 may be or include at least one computer program product having a set (e.g., at least one) of program modules, instructions, code or the like that is/are configured to carry out functions of embodiments described herein when executed by one or more processors. The described memory comprising working memory 120 and storage memory 130 can store an operating system and other computer programs, such as one or more computer programs/applications that execute to perform aspects described herein. Specifically, programs/applications can include computer readable program instructions that may be configured to carry out functions of embodiments of aspects described herein. Examples of/O devices 140 include but are not limited to microphones, speakers, Global Positioning System (GPS) devices, cameras, lights, accelerometers, gyroscopes, magnetometers, sensor devices configured to sense light, proximity, heart rate, body and/or ambient temperature, blood pressure, and/or skin resistance, a keyboard, a keypad, a pointing device, a display, and/or any other devices that enable a user to interact with computer system 500 and activity monitors.

Computer system 500 may communicate with one or more external device via one or more communication I/O interfaces 180. A network interface/adapter is an example I/O interface that enables computer system 500 to communicate with one or more networks, such as a local area network (LAN), a general wide area network (WAN), and/or a public network (e.g., the Internet), providing communication with other computing devices or systems, storage devices, or the like. Ethernet-based (such as Wi-Fi) interfaces and Bluetooth® adapters are just examples of the currently available types of network adapters used in computer systems (BLUETOOTH is a registered trademark of Bluetooth SIG, Inc., Kirkland, Washington, U.S.A.). The communication between communication I/O interfaces 180 and external devices can occur across wired and/or wireless communications link(s) such as Ethernet-based wired or wireless connections. Example wireless connections include cellular, Wi-Fi, Bluetooth®, proximity-based, near-field, or other types of wireless connections. More generally, communications link(s) may include one or more data storage devices, which may store one or more programs, one or more computer readable program instructions, and/or data, etc.

Computer system 500 may include and/or be coupled to and in communication with (e.g., as an external device of the computer system) removable/non-removable, volatile/non-volatile computer system storage media. For example, it may include and/or be coupled to a non-removable, non-volatile magnetic media (typically called a “hard drive”), solid state storage device, a magnetic disk drive for reading from and writing to a removable, non-volatile magnetic disk (e.g., a “floppy disk”), and/or an optical disk drive for reading from or writing to a removable, non-volatile optical disk, such as a CD-ROM, DVD-ROM or other optical media. Computer system 500 may be operational with numerous other general purpose or special purpose computing system environments or configurations. Computer system 500 may take any of various forms, well-known examples of which include, but are not limited to, personal computer (PC) system(s), server computer system(s), such as messaging server(s), thin client(s), thick client(s), workstation(s), laptop(s), handheld device(s), mobile device(s)/computer(s) such as smartphone(s), tablet(s), and wearable device(s), multiprocessor system(s), microprocessor-based system(s), telephony device(s), network appliance(s) (such as edge appliance(s)), virtualization device(s), storage controller(s), set top box(es), programmable consumer electronic(s), network PC(s), minicomputer system(s), mainframe computer system(s), and distributed cloud computing environment(s) that include any of the above systems or devices, and the like. Implant system 100, local system 200, artificial sensory system 300, remote system 400, and remote computing environments 1100A-110Z can include one or moe computer system (computing node) according to computer system 500.

The flowchart and block diagrams in the Figures illustrate the architecture, functionality, and operation of possible implementations of systems, methods, and computer program products according to various embodiments of the present disclosure. In this regard, each block in the flowchart or block diagrams may represent a module, segment, or portion of instructions, which comprises one or more executable instructions for implementing the specified logical function(s). In some alternative implementations, the functions noted in the blocks may occur out of the order noted in the Figures. For example, two blocks shown in succession may, in fact, be accomplished as one step, executed concurrently, substantially concurrently, in a partially or wholly temporally overlapping manner, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions. Aspects of the present invention may be a system, a method, and/or a computer program product, any of which may be configured to perform or facilitate aspects described herein. In some embodiments, aspects of the present invention may take the form of a computer program product, which may be embodied as computer readable medium(s). A computer readable medium may be a tangible storage device/medium having computer readable program code/instructions stored thereon. Example computer readable medium(s) include, but are not limited to, electronic, magnetic, optical, or semiconductor storage devices or systems, or any combination of the foregoing. Example embodiments of a computer readable medium include a hard drive or other mass-storage device, an electrical connection having wires, random access memory (RAM), read-only memory (ROM), erasable-programmable read-only memory such as EPROM or flash memory, an optical fiber, a portable computer disk/diskette, such as a compact disc read-only memory (CD-ROM) or Digital Versatile Disc (DVD), an optical storage device, a magnetic storage device, or any combination of the foregoing. The computer readable medium may be readable by a processor, processing unit, or the like, to obtain data (e.g., instructions) from the medium for execution. In a particular example, a computer program product is or includes one or more computer readable media that includes/stores computer readable program code to provide and facilitate one or more aspects described herein. As noted, program instruction contained or stored in/on a computer readable medium can be obtained and executed by any of various suitable components such as a processor of a computer system to cause the computer system to behave and function in a particular manner. Such program instructions for carrying out operations to perform, achieve, or facilitate aspects described herein may be written in, or compiled from code written in, any desired programming language. In some embodiments, such programming language includes object-oriented and/or procedural programming languages such as C, C++, C #, Java, Python, etc. Program code can include one or more program instructions obtained for execution by one or more processors. Computer program instructions may be provided to one or more processors of, e.g., one or more computer systems, to produce a machine, such that the program instructions, when executed by the one or more processors, perform, achieve, or facilitate aspects of the present invention, such as actions or functions described in flowcharts and/or block diagrams described herein. Thus, each block, or combinations of blocks, of the flowchart illustrations and/or block diagrams depicted and described herein can be implemented, in some embodiments, by computer program instructions. Although various embodiments are described above, these are only examples. For example, computing environments of other architectures can be used to incorporate and use one or more embodiments.

As noted, computer systems herein including computer systems defining remote system 400 can be defined in a cloud computing environment. Cloud computing is a model for enabling ubiquitous, convenient, on-demand network access to a shared pool of configurable computing resources (e.g., networks, servers, storage, applications, and services) that can be rapidly provisioned and released with minimal management effort or service provider interaction. This cloud model can be composed of five baseline characteristics, three service models, and four deployment models. Baseline Characteristics—On-demandself-service. A consumer can unilaterally provision computing capabilities, such as server time and network storage, as needed automatically without requiring human interaction with each service provider. Broad network access. Capabilities are available over the network and accessed through standard mechanisms that promote use by heterogeneous thin or thick client platforms (e.g., mobile phones, tablets, laptops, and workstations). Resource pooling. The provider's computing resources are pooled to serve multiple consumers using a multi-tenant model, with different physical and virtual resources dynamically assigned and reassigned according to consumer demand. There can be a sense of location independence in that the customer generally has no control or knowledge over the exact location of the provided resources but may be able to specify location at a higher level of abstraction (e.g., country, state, or datacenter). Examples of resources include storage, processing, memory, and network bandwidth. Rapid elasticity. Capabilities can be elastically provisioned and released, in some cases automatically, to scale rapidly outward and inward commensurate with demand. To the consumer, the capabilities available for provisioning often appear to be unlimited and can be appropriated in any quantity at any time. Measured service. Cloud systems automatically control and optimize resource use by leveraging a metering capability (Typically this can be done on a pay-per-use or charge-per-use basis) at some level of abstraction appropriate to the type of service (e.g., storage, processing, bandwidth, and active user accounts). Resource usage can be monitored, controlled, and reported, providing transparency for both the provider and consumer of the utilized service. Service Models—Software as a Service (SaaS). The capability provided to the consumer is to use the provider's applications running on a cloud infrastructure. A cloud infrastructure can include a collection of hardware and software that enables the five essential characteristics of cloud computing. The cloud infrastructure can be viewed as containing both a physical layer and an abstraction layer. The physical layer consists of the hardware resources that are necessary to support the cloud services being provided, and typically includes server, storage and network components. The abstraction layer consists of the software deployed across the physical layer, which manifests the essential cloud characteristics. Conceptually the abstraction layer sits above the physical layer. The applications are accessible from various client devices through either a thin client interface, such as a web browser (e.g., web-based email), or a program interface. The consumer does not manage or control the underlying cloud infrastructure including network, servers, operating systems, storage, or even individual application capabilities, with the possible exception of limited user-specific application configuration settings. Platform as a Service (PaaS). The capability provided to the consumer is to deploy onto the cloud infrastructure consumer-created or acquired applications created using programming languages, libraries, services, and tools supported by the provider. This capability does not necessarily preclude the use of compatible programming languages, libraries, services, and tools from other sources. The consumer does not manage or control the underlying cloud infrastructure including network, servers, operating systems, or storage, but has control over the deployed applications and possibly configuration settings for the application-hosting environment. Infrastructure as a Service (IaaS). The capability provided to the consumer is to provision processing, storage, networks, and other fundamental computing resources where the consumer is able to deploy and run arbitrary software, which can include operating systems and applications. The consumer does not manage or control the underlying cloud infrastructure but has control over operating systems, storage, and deployed applications; and possibly limited control of select networking components (e.g., host firewalls). Deployment Models—Private cloud. The cloud infrastructure can be provisioned for exclusive use by a single organization comprising multiple consumers (e.g., business units). It may be owned, managed, and operated by the organization, a third party, or some combination of them, and it may exist on or off premises. Community cloud. The cloud infrastructure can be provisioned for exclusive use by a specific community of consumers from organizations that have shared concerns (e.g., mission, security requirements, policy, and compliance considerations). It may be owned, managed, and operated by one or more of the organizations in the community, a third party, or some combination of them, and it may exist on or off premises. Public cloud. The cloud infrastructure can be provisioned for open use by the general public. It may be owned, managed, and operated by a business, academic, or government organization, or some combination of them. It exists on the premises of the cloud provider. Hybrid cloud. The cloud infrastructure can be a composition of two or more distinct cloud infrastructures (private, community, or public) that remain unique entities, but are bound together by standardized or proprietary technology that enables data and application portability (e.g., cloud bursting for load balancing between clouds).

The present disclosure is in the fields of neuroscience, biomedical engineering, materials science, and nanophotonics, and relates to the use of optogenetics to alter inner brain visual neurons to express light-sensitive proteins and become photosensitive cells within the brain, restoring visual perception and various aspects of vision when contacted with light from one or more emitters. Embodiments herein recognize that the International Agency for the Prevention of Blindness expects 196 million people to suffer from macular degeneration worldwide this year. Millions more suffer from traumatic ocular injury or glaucoma or other foveal defects. The result is foveal blindness⁹ in 3-5% of the global population. Retinal implants can help only a fraction of patients, and no therapy exists to restore foveal vision at the highest attainable acuity.^{1,10}

Embodiments herein recognize that vision normally begins when photoreceptors inside the back of the eye convert light signals to electrical signals that are then relayed through second- and third-order retinal neurons and the optic nerve to the lateral geniculate nucleus and, then to the visual cortex where visual images are formed (Baylor, D, 1996, Proc. Natl. Acad. Sci. USA 93:560-565; Wassle, H, 2004, Nat. Rev. Neurosci. 5:747-57). The severe loss of photoreceptor cells can be caused by congenital retinal degenerative diseases, such as retinitis pigmentosa (RP) (Sung, C H et al., 1991, Proc. Natl. Acad. Sci. USA 88:6481-85; Humphries, P et al., 1992, Science 256:804-8; Weleber, R G et al., in: S J Ryan, Ed, Retina, Mosby, St. Louis (1994), pp. 335-466), and can result in complete blindness. Age-related macular degeneration (AMD) is also a result of the degeneration and death of photoreceptor cells, which can cause severe visual impairment within the centrally located best visual area of the visual field. As photoreceptors die or become deficient in subjects, blindness may result as little or no signal is sent to the brain for further processing.

Embodiments herein recognize that prior art of interest includes U.S. Pat. No. 9,730,981 (herein incorporated entirely by reference) to Zhuo-Hua Pan, et al. relating to restoration of visual responses by in vivo delivery of rhodopsin nucleic acids. In Pan et al.'s project, nucleic acid vectors encoding light-gated cation-selective membrane channels, in particular channelrhodopsin-2 (Chop2), converted inner retinal neurons to photosensitive cells in photoreceptor-degenerated retina in an animal model. However, the methods focus on altering retinal neurons within the eye by a viral based gene therapy method, and do not extend to altering neurons downstream of the optic nerve such as in the brain or near the lateral geniculate nucleus (LGN) afferents in the foveal region of vision. Accordingly, the method is deficient in that it relies on light to enter the eye to contact the retina for sight and is not directed at exciting neurons within the brain or near the lateral geniculate nucleus (LGN) afferents in the foveal region of vision. Further, the method is deficient in that it is not directed to treating conditions where optic nerve damage results in blindness or reduced vision.

Embodiments herein recognize that thus, there is a continuing need for methods, compositions, and devices for restoring visual perception and various aspects of vision.

In embodiments, the present disclosure includes a method of restoring foveal vision, including: altering a first location of a neuron in a visual pathway of a patient in need thereof to form a light-emitting first location; and photostimulating the light-emitting first location to evoke neural responses which propagate along the neuron in the visual pathway, wherein the neural responses are formed with a light signal. In embodiments, the light signal is emitted from a synthetic source such as a semiconductor device. In embodiments, the first location includes neurons genetically encoded with one or more channelrhodopsin proteins to form photoreceptor cells within the first location. In embodiments, the first location is downstream of the optic nerve such as in the brain or near the lateral geniculate nucleus (LGN) afferents in the foveal region of vision. In some embodiments, the first location is one or more individual LGN ON- vs. OFF-channel modules entering the primary visual area (V1) of the cerebral cortex.

In some embodiments, the present disclosure includes a method of treating a subject for ocular disorder, including: administering an effective amount of composition to a subject to alter one or more first locations of one or more neurons in a visual pathway to form a plurality of light-emitting first locations; and photostimulating the plurality of light-emitting first locations to evoke neural responses which propagate along the neuron in the visual pathway to improve or form vision. In embodiments, the one or more first locations includes neurons genetically encoded with one or more channelrhodopsin proteins to form photoreceptor cells within the one or more first locations. In embodiments, the one or more first locations are downstream of the optic nerve such as in the brain or near the lateral geniculate nucleus (LGN) afferents in the foveal region of vision. In some embodiments, the one or more first locations are at one or more individual LGN ON- vs. OFF-channel modules entering V1.

In some embodiments, the present disclosure includes a method of mapping lateral geniculate nucleus (LGN) afferents in the foveal region of vision. In embodiments, mapping lateral geniculate nucleus (LGN) afferents in the foveal region of vision forms a map of LGN ON- and OFF-channel afferents to the primary visual cortex (V1). In embodiments, the one or more first locations including neurons are genetically modified to encoded with one or more channelrhodopsin proteins to form photoreceptor cells within the one or more first locations. Subsequent to the genetic modification, the one or more first locations are contacted with light to form a light signal and mapped. In embodiments, a plurality of light signals are plotted to form a map of one or more individual LGN ON- vs. OFF-channel modules entering V1.

In embodiments, a semiconductor device includes a substrate including one or more arrays of emitters/detectors spaced with about 225 to 275 μm, about 250 μm, or 250 μm pitch for patterning to target individual LGN input modules into V1 without unwanted targeting of adjacent hypercolumns. In embodiments, stimulation is obtained without spatial gaps in retinotopic coverage.

In other embodiments a system includes a variable-intensity light source; an emitter assembly in communication with the variable intensity light source, the emitter assembly including: a switch matrix including: a plurality of waveguides in communication with the variable-intensity light source for receiving a light generated by the variable-intensity light source; and a plurality of optical switching devices positioned between and in communication with the plurality of waveguides, at least one of the plurality of optical switching devices receiving the light generated by the variable-intensity light source from one of the plurality of waveguides and providing the light to a distinct one of the plurality of waveguides based on a desired operation of the emitter assembly; a plurality of optical modulation devices in communication with the plurality of waveguides of the switch matrix, each of the plurality of optical modulation devices receiving and modulating the light generated by the variable-intensity light source; and a plurality of emitter devices in communication with a corresponding optical modulation device of the plurality of optical modulation devices, each of the plurality of emitter devices emitting the provided light generated by the variable-intensity light source toward a plurality of LGN-Channelrhodopsin neurons to stimulate light-emitting cortical neurons in communication with the plurality of LGN-Channelrhodopsin neurons; and a detector assembly positioned adjacent the emitter assembly, the detector assembly including: a plurality of semiconductor detector devices positioned adjacent each of the plurality of emitter devices of the emitter assembly and the plurality of stimulated light-emitting cortical neurons, each of the plurality of semiconductor detector devices detecting photons generated by the stimulated light-emitting cortical neurons; and a plurality of optical filtration devices disposed over each of the plurality of semiconductor detector devices, each of the plurality of optical filtration devices allowing a distinct, predetermined wavelength of the photons generated by the stimulated light-emitting cortical neurons to pass to the corresponding semiconductor detector device.

The illustrative aspects of the present disclosure are designed to solve the problems herein described and/or other problems not discussed.

The patent or application file contains at least one drawing executed in color. Copies of this patent or patent application publication with color drawing(s) will be provided by the Office upon request and payment of the necessary fee. These and other features of this disclosure will be more readily understood from the following detailed description of the various aspects of the disclosure taken in conjunction with the accompanying drawings that depict various embodiments of the disclosure, in which:

FIGS. 2A-2D depict ON/OFF LGN input hypercolumns in V1. FIG. 2A depicts 1 cm² of V1. Left/right (blue/red) OD bands with simulated ON-OFF columns. The vertical meridian is oriented along the left edge of this image. FIG. 2B depicts a cartoon of the LGN afferent ON-OFF column map in layer 4. The oblong light and dark ovals represent the oblong ON and OFF LGN afferent bouton fields that input into layer 4 at each retinotopic position. The patterned layout shown is a hypothetical map based on recent discoveries about the functional anatomy of ON and OFF columns in area V1.³ The ON-OFF map predicts all ocular dominance (OD) and orientation selectivities, and is therefore the primary organizing principle of area V1. FIG. 2C depicts a close-up view of a cluster of four hypercolumns (ON and OFF in both eyes at a particular retinotopic position), from panel B. Note that each ON or OFF column tends to be 0.5 mm from its same-sign partner in the other eye, whereas the neighboring same-sign column in retinotopic space-within the same OD column—is 1 mm. FIG. 2D depicts a hypercolumns as in FIG. 2C, but here the identical-colored squares indicate the antagonistic ON and OFF columns for a given point in retinotopic space. These fundamental patterning principles form the basis of V1's map, and thus define targeting objectives of the present disclosure.

FIG. 2E-H depicts long-term 2P fluorescence imaging from non-human primates (NHPs). FIG. 2E depicts left, orientation tuning curves of neurons (N=148) on Day 158 (post-chamber implantation). Orange=average response from all neurons. Grey=preferred orientation responses from individual neurons, rotated to align at zero degrees. Curves were fit with a circular Gaussian. Right, Orientation tuning curves from four sample cells. FIG. 2F depicts left, cortical position of the cells on Day 158 as a function of their orientation preference (colors). Image brightness represents the average response strength. Right, orientation pinwheel structure of this cortical area. FIG. 2G depicts left, same as FIG. 2E, collected on Day 292 (111 identified neurons were recovered). Right, orientation tuning curves from the same four cells in A. FIG. 2G depicts same as FIG. 2F, collected on Day 292, revealing long-term reproducibility of cellular and columnar level functional circuit measurements.

FIGS. 3A-3C depicts all-optical interrogation of a V1 neuronal population in awake NHP VI. FIG. 3A depicts 2P image of V1 neurons expressing C1V1 and GCaMP6s. The colored regions of interest (ROIs) indicate neurons that responded to both visual and optical stimuli, targeted for further analysis. FIG. 3B depicts top, a differential image of GCaMP6s fluorescence (stimulated-baseline [F-F0], averaged across all stimulations), driven by visual stimuli consisting of gratings or colored patches. Bottom, calcium signals from 10 neurons (colors from panel A) in response to 9 varied visual stimuli (presentation times in gray). FIG. 3C depicts top, widefield optogenetic stimulation (0.8 mW/mm², 30 Hz and 25% duty ratio) evoked robust responses in the same neurons. Bottom, 8 sequential identical optogenetic stimulations evoked equivalent responses in each cycle.

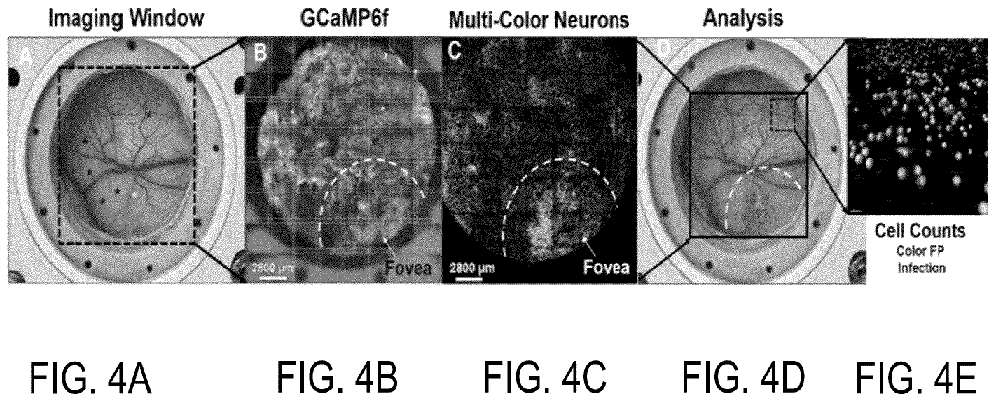

FIGS. 4A-4E depict data from NHP V1 convection-enhanced delivery (CED) of viruses, 2P Ca Imaging, and multi-color Aeq-FPs. FIG. 4A depicts an NHP 2 cm-diameter V1 imaging window in an implant design of the present disclosure (see FIG. 5). Black stars indicate the position of cortical CED injection sites and a previous electrode recording scar. The white star indicates the LGN CED injection scar. FIG. 4B depicts GCaMP6 Ca maximum projection image from 61,017 neurons stitched together from a 7×7 tiled array of 2800 μm-square 500 μm-deep image stacks (made with an Olympus 4×0.28 NA objective). Note the even GCaMP6 filling throughout the chamber. FIG. 4C depicts multi-color FP imaging revealing that the entire chamber was transfected, with the fovea showing extra density due to the yellow YFP-tagged ChR2-transfected boutons from the LGN inputs in fovea. These are 2P images of LGN inputs into cortex, and 2P images of the visual fovea in NHP V1 in accordance with the present disclosure. FIG. 4D depicts a color analysis, of computationally tagged cell bodies with colored spheres labeled based on their activation of the red, yellow, green, and blue channels of the 4-channel Prairie Ultima IV 2P microscope. Inset: magnified view chromatically identified cells.⁷

FIGS. 5A-5E depict a novel macaque NHP chamber in accordance with the present disclosure. FIG. 5A depicts where macaques receive CT/MR structural scanning to segment bone from brain areas in high-resolution (shown here with a radiolucent headpost attached). FIG. 5B depicts a 3D model of implants (cyan) are fit to each skull's (blue) precise contours so that the imaging window will lay flat against the target cortical imaging region. FIG. 5C depicts convection enhanced delivery of AAVs to cortical and subcortical areas through the imaging window fills up to 10 cm³ per injection. FIG. 5D depicts printed or machined implants fit-tested against skull models, with injection testing through the imaging window into custom phantom engineered silicone brains having the same Young's Modulus as a real brain. FIG. 5E depicts a chamber design in accordance with the present disclosure that achieves the innovations listed above with the central design feature being the silicone skirt engineered with the same Young's Modulus as the brain, so that the coverslip is always pressed against the brain (discouraging biofilm growth and promoting imaging window patency for the long-term), while not allowing high pressure to build up with normal brain swelling or motion that could lead to neurodegeneration. FIG. 5F depicts Instron tensile measurements of engineered silicone Young's Modulus for various recipes (C=Catalyst; P=Polymer; O=Oil). Catalyst ratio has a non-linear effect on Young's Modulus. FIG. 5G depicts data of an implanted macaque chamber with a 2 cm wide imaging window. Stars indicate injection points or previous electrode insertion scars.

FIGS. 6A-6D depict a conceptual approach to optogenetic inverse modeling of cortical functional architecture in the blind in accordance with the present disclosure. FIG. 6A conceptually shows stimulating each point of the layer 4 LGN afferent map optogenetically, evokes VSD responses: but only those points at the center of a layer 4 ON and OFF afferent input domain will generate strong responses (other points will cancel or weaken due to ON and OFF intermixing and splitting stimulation across OD columns). The map of the present disclosure will not indicate the contrast-sign of the columns, however. FIG. 6B conceptually depicts by conducting reverse-correlation mapping with an optimized spatiotemporal optogenetic mapping stimuli, points sharing the same contrast-sign will be identified, though it will not yet be known which population indicates ON vs OFF to the subject such as a NHP. FIG. 6C conceptually depicts patterning (and other) clues that will guide the mapping. For example, because OFF columns are known to be more numerous than ON columns, OFF vs ON contrast-signs will be assigned if it is determined that a difference in numerosity is present. FIG. 6D conceptually depicts, following from FIG. 3, a fundamental patterning and spacing of the ON and OFF columns that will be used to determine the OD organization. Further, because layer 4 afferent fields are oblong in the axis along the OD columns (See FIGS. 3B-3B), they have paired and mutually inhibitory ON and OFF columns sharing the same retinotopic positions. They also run perpendicular to the ON and OFF stripes in FIG. 6C, which will be further exploited to determine the OD column map.

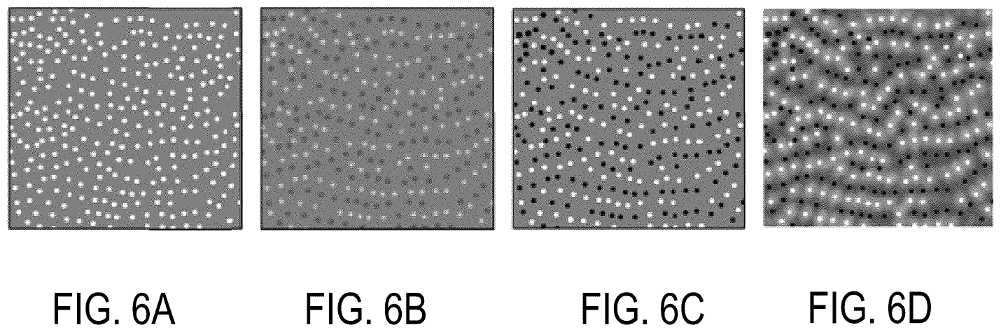

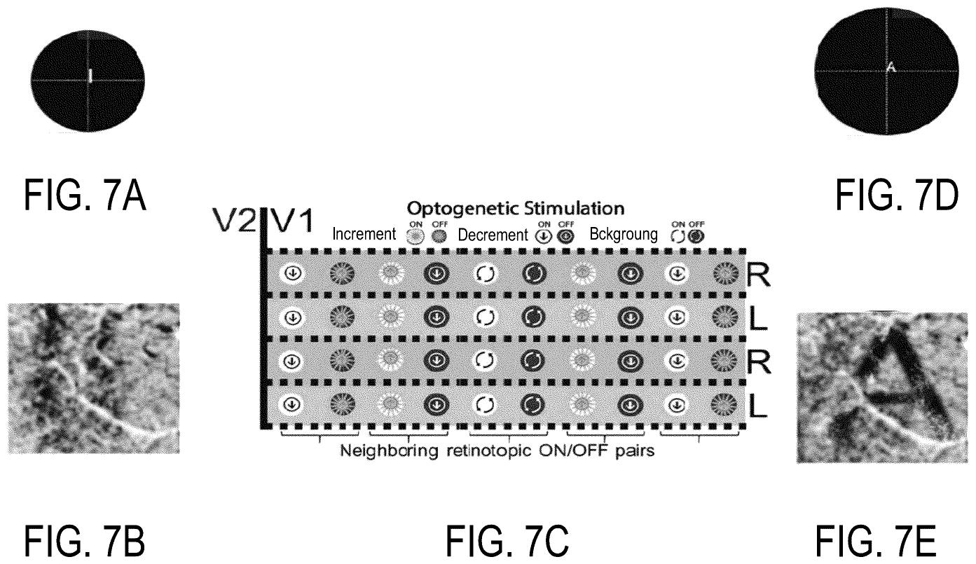

FIGS. 7A-7E depict optogenetic stimulation in accordance with embodiments of the present disclosure. FIG. 7 A depicts a vertical line stimulus, presented at the fovea. FIG. 7B depicts data¹ of intrinsic signal optical imaging of a 1 cm² field in V1 recorded from a subject such as an NHP. V1 activates to the two edges of the vertical line segment as if they were individual stripes (one for each edge of the line). Note that intrinsic signal (same physiological mechanisms as BOLD) does not differentiate between ON and OFF responses, whereas our model will. FIG. 7C depicts a cartoon of the hypothesized ON and OFF column activities within the Layer 4 LGN afferent map. FIG. 7D depicts if we were to present a letter ‘A’ optogenetically—at the resolution of a typical New York Times newspaper font—our model i suggests that the optogenetic stimulation would produce a cortical pattern simulated in FIG. 7E.

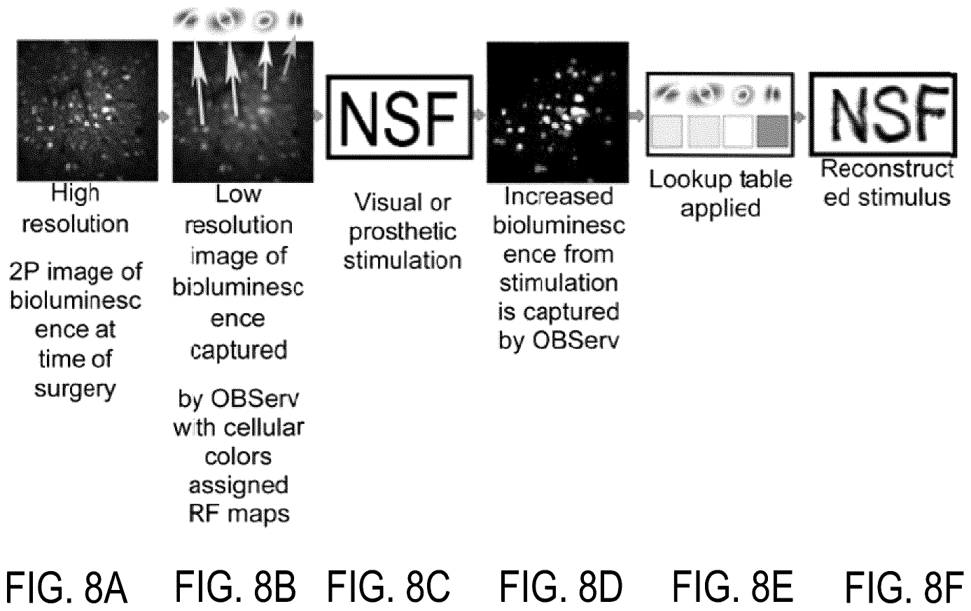

FIGS. 8A-8F depict a spatiochromatic read-out and decode schema of the present disclosure. FIG. 8A depicts bioluminescent cortical neurons in V1 imaged with high resolution 2P. FIG. 8B depicts the implant CCD capturing lower resolution images of bioluminescent neurons and their receptive fields (RF), allowing for the generation of a color lookup table that corresponds to neuronal orientation selectivity. FIGS. 8C-8D depict a stimulus of the present disclosure presenting and images of the implant capturing increased bioluminescence from stimulation. FIG. 8E depicts data from the color lookup table is correlated to the images from stimulation. FIG. 8F depicts eye movement, dimension reduction, and state-vector analyses of the images is conducted to reconstruct the original stimulus.

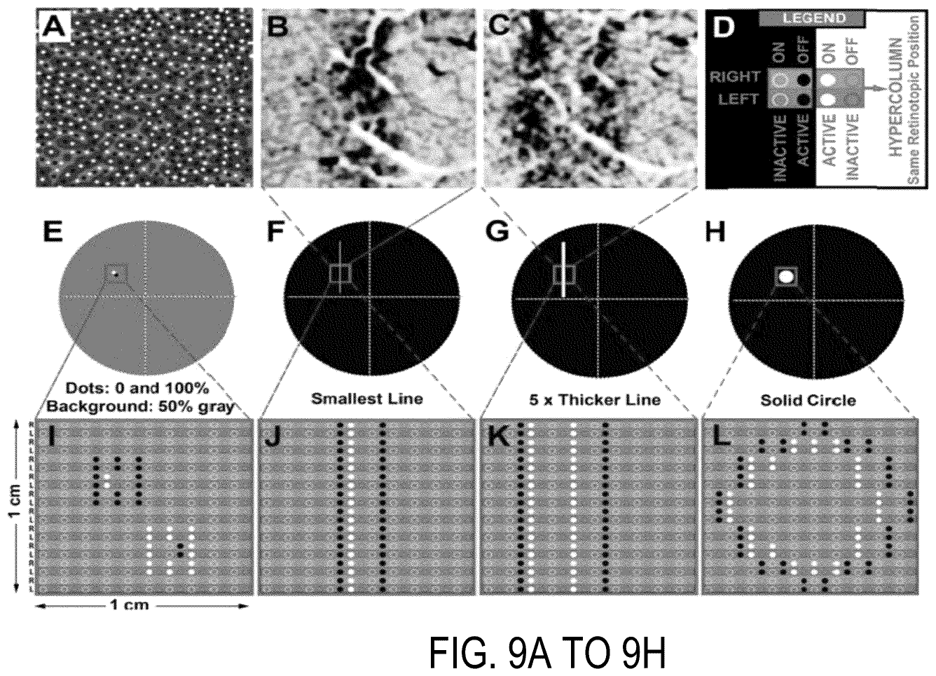

FIGS. 9A-9H depict V1 hypercolumn LGN-Input (V1, Layer 4) as the primary organizing principle of the early visual system. FIG. 9A depicts data: 1 cm² of subject such as NHP V1 ocular dominance columns imaged with intrinsic signal^{1,2}. (Note: white/black dots indicating ON/OFF column-centers are simulated, ocular dominance is real data). FIG. 9B depicts intrinsic signal representation of a 0.13° bar, which is at the resolution limit of vision at this eccentricity (stimulus at about 5° in the visual periphery as in panel F). FIG. 9C depicts the same bar, now 5× wider (0.64°) reveals that V1 processes only the edges of the bar. This is the first optical image of a surface's edge in isolation: a 1-D object (stimulus represented in panel FIG. 9G). FIG. 9D depicts a summary of how hypercolumns process visual edges in V1, following from recent findings^{3,4}. Each hypercolumn receives one region of LGN ON boutons and one region of LGN OFF boutons for each eye (L/R). The edge of a binocular white field activates the ON columns for both eyes in the hypercolumns on the white side of the edge, whereas inside the hypercolumn representing the position on the black side of the edge, only OFF domains are activated. The edges of all surfaces are thus encoded by the interplay of ON/OFF activity between neighboring hypercolumns spanning each visual edge. FIGS. 9E-9H depict various visual stimuli. FIGS. 9I to 9L depict the ON/OFF columnar activation patterns expected from the visual stimuli in FIGS. 9E-9H.

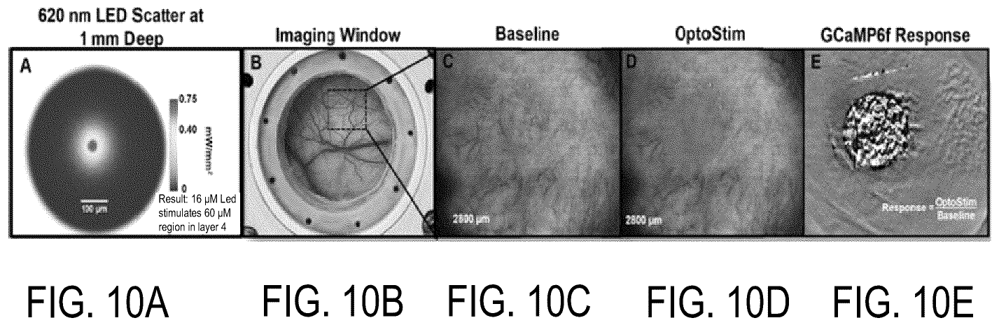

FIGS. 10A-10E depict 1P Optogenetic activation of LGN boutons in Layer 4, with Ca Imaging readout from V1 pyramidal neurons. FIG. 10A depicts a Zemax optical wavefront propagation model of macaque NHP V1 cortical red light transmission and scatter based on the measured light-scattering data from Acker et alf. The model shows that a 16 μm-wide laser of 620 nm light (matching OBServ's specs: the optimal ReaChR activation wavelength) having a 0.12 mW emission, from the surface of V1, with peak transmission in layer 4 (1 mm deep) will suffice in strength to activate ReaChR (we showed in Ref⁶ that irradiance >0.4 mW/mm² achieves stimulation of red-shifted ChR's). FIGS. 10B-10D depict the model tested in an NHP (who had ChR2, not ReaChR, which means that the test was >11× more stringent due to the decreased transmission of blue light in cortex-) using a DMD video 470 nm laser (here projecting a ˜3 mm spot whose position is indicated by a round blue circle), at a power level of 11 mW/mm² to optostimulate layer 4 LGN boutons deep within V1. FIG. 10E depicts GCaMP6f fluorescence Ca imaging of upper layer V1 cells was strongly driven by the LGN optostimulation. Note the lack of spreading of the V1 responses beyond the LGN activation beam width, suggesting high retinotopic precision with LGN optostimulation.

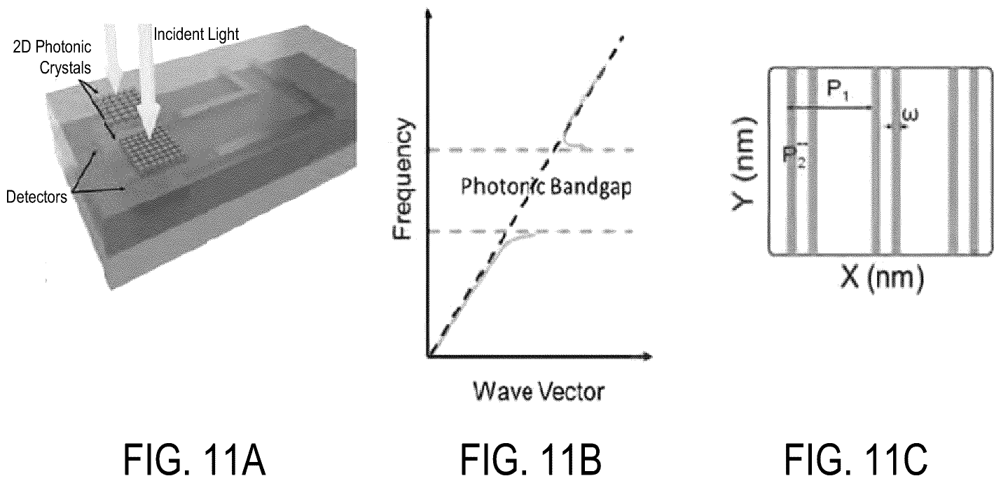

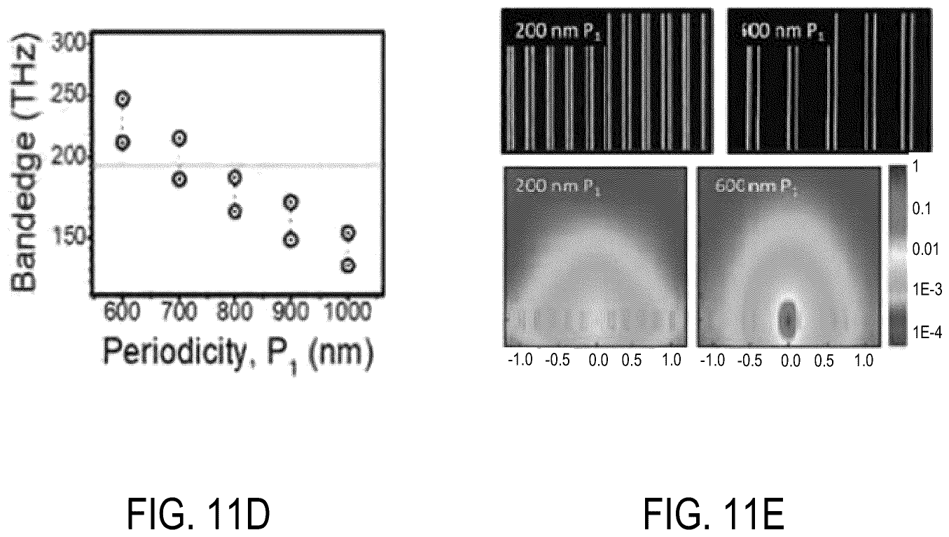

FIGS. 11A-11D depict an illustration of the PC spectrometer and detectors. FIG. 11B depicts an illustration of the photonic band in a 1D PC structure. FIG. 11 C depicts a top-down schematic of 1D PC with lattice periodicity P₁, sub-lattice periodicity P₂, and width w. FIG. 11 D depicts a corresponding photonic bandgap tuning for different P₁, computed using Finite-difference time-domain numerical calculations. E) Light emission intensity engineering in 1D PC via different P₁ periodicities.

FIG. 12A depicts a bioluminescent Tet-Bow analysis and the data cell count quantification revealed that 61, 017 cells were identified based on their shape (using Imaris software). Once the cells were identified, the average intensity in each of our 4 imaging channels was determined. Each channel's baseline and noise was calculated from the data, establishing the Signal:Noise (SNR). From this, the number of clearly discernible levels of chromatic activity in each channel were determined (discernibility was defined as bins of 3 SNR levels). Because scans were performed at only one wavelength (830 nm) and the system was more optimized for some channels than for others, it was determined that 9 levels of red, 11 levels of yellow, 8 levels of green, and 6 levels of blue signal were present. Thus, each cell obtained one of 4,752 combinations of R,Y,G,B. These results indicate that a mixed-titer FP approach succeeded, and achieved Brainbow-like stochastic colored labelling with thousands of discernible colors. This color space discernibility will rise with the hyperspectral hardware.

FIG. 12B depicts a bioluminescent color separation in HSV color space. Here the 61,017 imaged cells were displayed from FIG. 12A, now projected into HSV color space, which highlights the color separation achieved by our multi-color approach. Note that the widest separations are along the Blue-Yellow axis, due to the fact that only one 2P laser wavelength (830 nm) was used, which emphasizes 2P excitation of blue fluorophores. With the hyperspectral approach of the present disclosure, it is expected to fill the entire HSV space much more completely, perhaps with as many as one or more orders of magnitude more discernible colors.

FIG. 12C depicts A) high precision optical fiber coupling in accordance with the present disclosure. Fiber at the edge of the chip accurately couples light into the wave guide on the chip. FIG. 12C depicts B) a diagram of the single-photon microscope integrated with a 1-nm-resolution scanning stage and state-of-the-art single-photon detectors.

FIG. 13A depict chip layouts of the present disclosure. (Note: layouts indicate relative position and connectivity of components accurately, but are not drawn to scale, as the actual device size will be much smaller). FIG. 13A (top lefts photograph) depicts a fiber at the left edge of the chip couples light into the waveguide entering the 4×1 emitter/detector chip (alignment process in FIG. 12C). It is intended to illuminate a single emitter at a time, using a raster sequence to activate each emitter in turn (which will scale to arbitrarily sized arrays, just as in standard video projectors). The electronic control of the MRR cascade will determine the channeling of coherent light to the next device in the cascade. MZIs will produce PWM of the light entering each emitter, and emitter shape and size will be designed to produce specific lensless beam-forming optical modifications to pre-chirp the emitted light, to ameliorate the light scattering effects of ˜1 mm of depth of cortical tissue lying between the surface and the LGN boutons (which are most highly concentrated in layer 4). This will optimize focus and distribution of the 250 um optogenetic activation spot. In the event that our experiments determine that we need to illuminate more than one emitter at a time, our design will allow for that approach without modification. Copper wiring indicates the control and I/O schema, including how data from the detector chiplet (light grey) will be connected to the underlying emitter chip (copper wire bonds on right edge of panel). Insets: Scanning Electron Microscope images of actual nanoscale devices produced in fabs.⁸ FIG. 13A (top right) depicts a layout of 4×4 emitter-detector dyads at 250 um pitch in both dimensions for chips of the present disclosure, including connections from detector chiplets to underlying emitter chips (not to scale; I/O connections and MRR cascade not shown for clarity). Inset: magnified view showing relationship of emitters to detectors (which sit on elevated chiplets).

FIG. 13B depicts testing of the present disclosure. FIG. 13B depicts: NHPs will fixate a red cross in a 2° window, to trigger the start of a trial. After 300 ms, the fixation target will vanish and the NHP will continue to fixate the remembered location. Either sparse noise or oriented gratings will appear while recording 2P Ca+ responses. FIG. 13B depicts calibrating the causal prosthetic neurometric curve (blue) to the visual stimulation neurometric curve (red) based on the Ca+ responses in each cell.

FIG. 13C depicts a causal model of the transform between LGN Inputs and V1 Orientation preference of the present disclosure. FIG. 13C (left) depicts a cartoon of a single layer 4 LGN input hypercolumn (Green square) with one ON (white) and one OFF (black) input module for each eye (L/R=Blue/Red). FIG. 13C (middle) depicts an oriented edge in visual space is perceived when an OFF module in one hypercolumn is co-activated with an ON module in the adjacent hypercolumn via optostimulation. A V1 cell positioned between these two modules on the cortical surface will receive dendritic inputs from both modules to form a receptive field that has (in this example) a horizontal orientation preference.^{3,4} Orientation selectivity is further refined through recurrent horizontal connections, with neighboring collinear V1 cells having the same preference. Thus, orientation preference is ultimately derived by the matched and correlated inputs of adjacent LGN inputs.

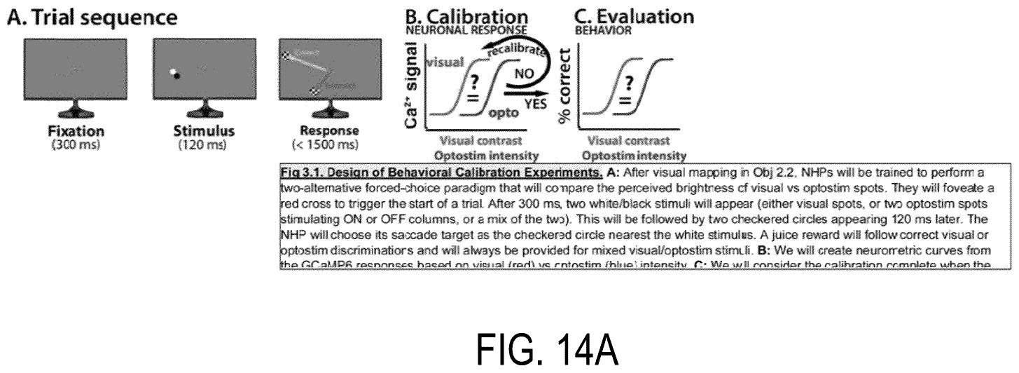

FIG. 14 depict a design of behavioral calibration experiments of the present disclosure. FIG. 14 depicts after visual mapping of the present disclosure, NHPs will be trained to perform a two-alternative forced-choice paradigm that will compare the perceived brightness of visual vs optostim spots. They will foveate a red cross to trigger the start of a trial. After 300 ms, two white/black stimuli will appear (either visual spots, or two optostim spots stimulating ON or OFF columns, or a mix of the two). This will be followed by two checkered circles appearing 120 ms later. The NHP will choose its saccade target as the checkered circle nearest the white stimulus. A juice reward will follow correct visual or optostim discriminations and will always be provided for mixed visual/optostim stimuli. FIG. 14 (calibration neuronal response) depicts neurometric curves from the GCaMP6 responses based on visual (red) vs optostim (blue) intensity. FIG. 14 (evaluation behavior) depicts evaluation behavior and the calibration completeness when the psychometric (behavioral response) curves between visual and optostim paradigms match.

FIG. 15A depicts a schematic view of a system including an emitter assembly and a detector assembly, according to embodiments of the disclosure.

FIG. 15B depicts a schematic view of a switch matrix of the emitter assembly of FIG. 15A, according to embodiments of the disclosure.

FIG. 15C depicts a schematic view of a portion of the emitter assembly of FIG. 15A including emitter devices, according to embodiments of the disclosure.

FIG. 15D depicts a side cross-sectional schematic view of the system of FIG. 15A, according to embodiments of the disclosure.

It is noted that the drawings of the disclosure are not necessarily to scale. The drawings are intended to depict only typical aspects of the disclosure, and therefore should not be considered as limiting the scope of the disclosure. In the drawings, like numbering represents like elements between the drawings.

Embodiments of the present disclosure drive stimulation in the primary visual cortex (V1) by activating thalamic (lateral geniculate nucleus; LGN) neuronal afferents entering V1 with synaptic precision, as in natural vision. Accordingly, the present disclosure relates to formulations, methods and devices for the restoration of visual responses, reducing or prevenfing the development or the risk of ocular disorders, and/or alleviating or curing ocular disorders including blindness in a subject such as a human or other non-human mammal or other animal.

In embodiments, ocular disorders suitable for treatment in accordance with the present disclosure include one which involves one or more deficient photoreceptor cells in the retina, as well as deficiencies to the optic nerve. Non-limiting examples of ocular disorders include; developmental abnormalities that affect both anterior and posterior segments of the eye; anterior segment disorders including glaucoma, cataracts, corneal dystrophy, keratoconus; posterior segment disorders including blinding disorders caused by photoreceptor malfunction and/or death caused by retinal dystrophies and degenerations; retinal disorders including congenital stationary night blindness; age-related macular degeneration; congenital cone dystrophies; and a large group of retinitis-pigmentosa (RP)-related disorders. These disorders include genetically pre-disposed death of photoreceptor cells, rods and cones in the retina, occurring at various ages. Among those are severe retinopathies, such as subtypes of RP itself that progresses with age and causes blindness in childhood and early adulthood and RP-associated diseases, such as genetic subtypes of LCA, which frequently results in loss of vision during childhood, as early as the first year of life. The latter disorders are generally characterized by severe reduction, and often complete loss of photoreceptor cells, rods and cones. (Trabulsi, E I, ed., Genetic Diseases of the Eye, Oxford University Press, N Y, 1998).

In embodiments, methods of the present disclosure are useful for the treatment and/or restoration of at least partial vision to subjects that have lost vision due to ocular disorders, as well as damage to the optic nerve. It is anticipated that these disorders, as well as blinding disorders of presently unknown causation which later are characterized by the same description as above, may also be successfully treated by this method. Thus, the particular ocular disorder treated by methods of the present disclosure may include the above-mentioned disorders and a number of diseases which have yet to be so characterized.

In embodiments, methods of the present disclosure include administering to a subject in need thereof an effective amount of a composition suitable for altering neural cells to express photosensitive membrane-channels or molecules within the brain or near the lateral geniculate nucleus (LGN) afferents in the foveal region of vision by a gene therapy method, and illuminating and/or stimulating altered neurons by a semiconductor based light emitter pre-positioned to send a plurality of light signals to the altered neural cells.

In some embodiments, treatments of the present disclosure for vision loss or blindness include expressing photosensitive membrane-channels or molecules within the brain or near/upon the lateral geniculate nucleus (LGN) afferents in the foveal region by a viral based gene therapy method, and stimulating the altered neurons with light not obtained from the eye to restore or generate visual responses.

Advantages of embodiments of the present disclosure include obtaining a permanent treatment of the vision loss or blindness with high spatial and temporal resolution for the restored vision. Embodiments of the present disclosure also advantageously includes integrated nanophotonics technologies (design, chip fabrication, and packaging), for the precise causal control of cortical circuits required for neural prosthetics in a subject's brain to serve as a cortical brain stimulation technology. Systems of the present disclosure will drive stimulation in the primary visual cortex (V1) by activating thalamic (lateral geniculate nucleus; LGN) neuronal afferents entering V1 with synaptic precision, as in natural vision.

In embodiments, the methods of the present disclosure and devices are configured to independently target the individual LGN ON- vs. OFF-channel modules entering V1, using advanced beamforming nanophotonics to achieve optimized optogenetic stimulation, because co-activation of unwanted targets in neighboring antagonistic modules—a common problem with current electrode technologies—will result in reduced perceived prosthetic contrast and resolution. Whereas other all-optical strategies are under development, no extant devices will generate optimized and naturalistic spatiotemporal cortical stimulation patterns with full feedback gain control.

In embodiments, the present disclosure includes one or more nanoscale 630 nm coherent light emitter devices optimized in quantum-efficiency. Light-scattering will be accounted for using beamforming nanotechnology to achieve deep cortical optogenetic stimulation of the LGN afferents. Embodiments include hyperspectral devices configured to detect responses from an innovative multicolor bioluminescence calcium indicator system, genetically encoded into V1 neurons, to provide feedback to control prosthetic gain in real-time. Embodiments, further include characterized and scalable implantable photonic emitter/detector devices that are calibrated and optimized for a subject such as a non-human primate (NHP) cortex, which can then be configured in any arrangement for any cortical region.

The combination of new advances to the all-optical interrogation methods, optogenetic analysis methods, ultra-large field two-photon imaging, and integrated photonics will bring much needed ground-truth to the understanding of the role and mechanisms of cortical visual processing, and the utility of nanophotonics approaches to brain-machine interfaces. Accordingly, the present disclosure also provides cortical visuocognitive prosthetics.

As used in the present specification, the following words and phrases are generally intended to have the meanings as set forth below, except to the extent that the context in which they are used indicates otherwise.

As used herein, the singular forms “a”, “an”, and “the” include plural references unless the context clearly dictates otherwise. Thus, for example, references to “a compound” include the use of one or more compound(s). “A step” of a method means at least one step, and it could be one, two, three, four, five or even more method steps.

As used herein the terms “about,” “approximately,” and the like, when used in connection with a numerical variable, generally refers to the value of the variable and to all values of the variable that are within the experimental error (e.g., within the 95% confidence interval [CI 95%] for the mean) or within ±10% of the indicated value, whichever is greater.

As used herein, subject may include, but is not limited to humans, and animals, e.g., rhesus monkeys, macaques, and other monkeys.

As used herein, substrate means a material subjected to micro- and/or nanofabrication for example any material including but not limited to polymeric, ceramic, metallic, semiconductor, composite material, silicon, silicon oxide, germanium, or the like.

As used herein, the terms “polypeptide sequence” and “amino acid sequence” are used interchangeably.

As used herein the term “prevent”, “preventing” and “prevention” of ocular disorder means (1) reducing the risk of a patient who is not experiencing symptoms of ocular disorder from developing ocular disorder, or (2) reducing the frequency of, the severity of, or a complete elimination of ocular disorder in a subject.

As used herein the term “therapeutically effective amount” means the amount of a compound that, when administered to a subject for treating or preventing ocular disorder, is sufficient to have an effect on such treatment or prevention of the ocular disorder. A “therapeutically effective amount” can vary depending, for example, on the compound, the severity of the ocular disorder, the etiology of the ocular disorder, comorbidities of the subject, the age of the subject to be treated and/or the weight of the subject to be treated. A “therapeutically effective amount” is an amount sufficient to alter the subjects' natural state.

In embodiments, the present disclosure relates to a method of altering cortical visual processing, including: altering a first location of a neuron in a visual pathway of a patient in need thereof to form a light-emitting first location; and photostimulating the light-emitting first location to evoke neural responses which propagate along the neuron in the visual pathway, wherein the neural responses are formed or modulated with a light signal. In embodiments, the first location includes neurons genetically encoded with one or more channelrhodopsin proteins. In embodiments, the first location includes neurons genetically encoded with one or more channelrhodopsin proteins to form photoreceptor cells within the first location. In embodiments, the first location is downstream of the optic nerve such as in the brain or near the lateral geniculate nucleus (LGN) afferents in the foveal region of vision. Accordingly, the methods do not rely on light entering the eye to contact the retina for sight and excite neurons within the brain or near the lateral geniculate nucleus (LGN) afferents in the foveal region of vision by emitted light, such as from a semiconductor device.

In some embodiments, the present disclosure relates to a method of restoring foveal vision, including: altering a first location of a neuron in a visual pathway of a patient in need thereof to form a light-emitting first location; and photostimulating the light-emitting first location to evoke neural responses which propagate along the neuron in the visual pathway, wherein the neural responses are formed or modulated with a light signal. In embodiments, the first location includes neurons genetically encoded with one or more channelrhodopsin proteins to form photoreceptor cells within the first location. In embodiments, the first location is downstream of the optic nerve such as in the brain or near the lateral geniculate nucleus (LGN) afferents in the foveal region of vision. In embodiments, the method further includes optogenetic neural stimulation from beamforming, coherent light emitter arrays. In embodiments, the method further includes optogenetic neural stimulation from beamforming, coherent light emitter arrays that optimize power calibration (prosthetic contrast gain control) by reading out genetically encoded bioluminescent cortical responses with a sensor made of p-i-n photodiodes for real-time feedback. In some embodiments, the first location is one or more individual LGN ON- vs. OFF-channel modules entering V1. In some embodiments, the methods further include sensing the evoked neural responses at a second location on a sensor, and analyzing the sensed neural responses to form data. In some embodiments, photostimulating further includes beaming nanophotonics to the first location to obtain optogenetic stimulation. In some embodiments, nanoscale 630 nm coherent light emitter devices are used to stimulate the first region. In some embodiments, hyperspectral devices are provided to detect responses from a multicolor bioluminescence calcium indicator system, which embodiments genetically encode into V1 neurons. In embodiments, photostimulating is performed under conditions sufficient to form naturalistic spatiotemporal cortical stimulation patterns. In embodiments, photostimulating is performed under conditions sufficient to control a light signal feedback and gain. In embodiments, the visual responses are evoked for the purposes of providing visibly perceptible information to the subject.

In some embodiments, the present disclosure includes a method of mapping lateral geniculate nucleus (LGN) afferents in the foveal region of vision. In embodiments, mapping lateral geniculate nucleus (LGN) afferents in the foveal region of vision forms a map of LGN ON- and OFF-channel afferents to the primary visual cortex (V1).

In embodiments, a device may be provided including arrays of emitters/detectors spaced with about 250 μm pitch or 250 μm pitch for patterning to target individual LGN input modules into V1 without unwanted targeting of adjacent hypercolumns. In embodiments, stimulation is obtained without spatial gaps in retinotopic coverage.

Embodiments of the present disclosure also include providing and preparing subjects such as non-human primates, by transducing optogenes into the LGN of the subject, with multicolor bioluminescent proteins transduced into a large >3 cm² field of V1, calibrated against two-photon calcium imaging of jGCaMP7 fluorescence from the same neurons. Embodiments also include empirically determining the scatter and penetration depth of coherent light emitter/detector devices in the NHP cortex.

In some embodiments, the present disclosure provides a hyperspectral imaging system that will record multicolor bioluminescent calcium responses from V1. In embodiments, LGN boutons are stimulated in a pattern that mimics naturalistic input. Embodiments will leverage advances in retinal implant technology to optimize our design for contrast sensitivity, acuity, and form vision. In some embodiments, a device is implanted over V1's foveal region, and may be configured to project visual information onto a specific set of excitatory neurons in the brain's hard-wired visual pathway.

Briefly turning to FIGS. 15A-15D, non-limiting examples of a system 1000 are shown. System 1000 may be used, for example, to interact and/or stimulate cortical neurons as discussed herein.

As shown in FIG. 15A, system 1000 may include an emitter assembly 1002, a detector assembly 1004, and a variable-intensity light source 1006. In the non-limiting example light source 1006 may be variable-intensity and/or may be configured to provide light or energy at various intensities. The intensities of light generated by light source 1006 may be dependent on a variety of factors including, but not limited to, the desired intensity for the light emitted by emitter assembly 1002, the function and/or operation of elements/devices in emitter assembly 1002 (e.g., MZI), and/or the number of emitters of emitter assembly 1002 that may emit the light generated by light source 1006 toward the subject (e.g., neurons). In non-limiting examples, light source 1006 may be formed as a single laser diode or a plurality of laser diodes capable of generating light at various intensities within system 1000.

In other non-limiting examples (not shown), light source 1006 may be formed as a single or plurality of microLED(s) configured to provide a light, as discussed herein. In the non-limiting example including microLED(s), the need of some portions of system 1000 (e.g., waveguides 1012) may not be required in order to provide the generated light to the emitter of system 1000 and/or neurons, as discussed herein.

Emitter assembly 1002 of system 1000 may be in communication with light source 1006. More specifically, and as shown in FIG. 15A emitter assembly 1002 may be positioned downstream of light source 1006 and may be (optically) coupled or in communication with light source 1006, such that emitter assembly 1002 may receive, process, and subsequently emit light from light source 1006. In the non-limiting example shown, emitter assembly 1002 may include a switch matrix 1008 in direct communication with light source 1006. Switch matrix 1008 may include a plurality of waveguides 1012 in communication with light source 1006 for receiving the light generated by light source 1006, and a plurality of optical switching devices 1010 positioned between and in communication with the plurality of waveguides 1012. More specifically, switch matrix 1008 may include a plurality of waveguides 1012 and optical switching devices 1010 that may be formed in series with one another in a “tree-type” configuration, such that a single optical switching device 1010 may coupled and/or be in communication with two distinct waveguides 1012 of switch matrix 1008. As discussed herein, switch matrix 1008 may be used in emitter assembly 1002 to move light from single light source 1006 to a desired emitter or emitters of the plurality of emitter devices 1020 included in emitter assembly 1002.

Turning briefly to FIG. 15B, with continued reference to FIG. 15A, light generated by light source 1006 may be initially received in an intake waveguide 10121. Two optical switching devices 1010A, 1010B may couple and/or be in communication with intake waveguide 1012I, as well as a subsequent, or downstream waveguide 1012A or 1012B. That is, optical switching device 1010A may be in communication with intake waveguide 1012I as well as waveguide 1012A, while optical switching device 1010B may be in communication with intake waveguide 10121 as well as waveguide 1012B—forming two separate “branches” in the “tree-type” configuration for switch matrix 1008 of emitter assembly 1002. In the non-limiting example shown, optical switching devices 1010A-1010F and waveguides 1012A-1012F may be formed and/or may be in communication in such a way that waveguides 1012C, 1012D, 1012E, 1012F all correspond to and/or provide light to a distinct emitter device 1020 of emitter assembly 1002, as discussed herein. As such, the number of optical switching devices 1010 and waveguides 1012 may be dependent, at least in part on the number of emitter devices 1020 included in emitter assembly 1002. In this example, the operation (e.g., opening/closing) of switching devices 1010 may determine where the generated light flows within emitter assembly 1002. That is, where optical switching devices 1010A and 1010C are open, light may flow to emitter device 1020A through intake waveguide 1012I, waveguide 1012A, and waveguide 1012C, respectively. Alternatively, where optical switching devices 1010A and 1010D are open, light may flow to emitter device 1020B through intake waveguide 10121, waveguide 1012A, and waveguide 1012D, respectively.

Optical switching device 1010 may be formed from any suitable device, component, or assembly that may selectively provide light within system 1000 based on command and/or a desired or predetermined operation of system 1000. For example, the plurality of optical switching devices 1010 may be formed as tunable micro-ring resonators (MRR). In the example, the MRRs may operate based on thermal-optics or electro-optics.

Returning to FIG. 15A, emitter assembly 1002 may also include a plurality of optical modulation devices 1018. More specifically, each single/final waveguide 1012 of switch matrix 1008 that may provide light to a corresponding emitter device 1020 may be in communication with a corresponding optical modulation devices 1018. Each optical modulation devices 1018 may be positioned between and may be in communication with both the single/final waveguide 1012, as well as a corresponding emitter device 1020 of emitter assembly 1002. That is, optical modulation devices 1018 may be positioned downstream of switch matrix 1008 but upstream of emitter device 1020. Optical modulation devices 1018 may receive, and when applicable, modulate light generated by light source 1006 before providing the (modulated) light to emitter device 1020. Modulation of the light by optical modulation devices 1018 may include modulating or adjusting the intensity of the light and/or adjusting the pulse width (e.g., pulse width modulation (PWM)) for the light prior to being emitted by emitter device 1020, as discussed herein. As such, optical modulation devices 1018 may be formed from any suitable device, feature, component, and/or assembly that may modulate light received from waveguides 1012 within emitter assembly 1002. In a non-limiting example, optical modulation devices 1018 may be formed as a tunable Mach-Zehnder interferometer (MZI). In an example, and similar to MRR forming switching devices 1010, MZIs forming optical modulation devices 1018 may operate based on thermal-optics or electro-optics.

Emitter assembly 1002 may also include a plurality of emitter devices 1020. Emitter devices 1020 may be in communication with corresponding optical modulation devices 1018. Each of the plurality of emitter devices 1020 may correspond to a single optical modulation devices 1018, as well as a single/final waveguide 1012 of switching matrix 1008 that may provide emitter device 1020 with light to be emitted toward, for example, a plurality of LGN-Channelrhodopsin neurons. For example, and returning to FIG. 15B, waveguide 1012C may be in communication with, may correspond to, and/or may provide light received therein to emitter device 1020A (through optical modulation devices 1018A, not shown), while waveguide 1012F may be in communication with, may correspond to, and/or may provide light received therein to emitter device 1020D (through optical modulation devices 1018D, not shown). Once received, (modulated) light may be emitted from emitter device 1020 toward a plurality of LGN-Channelrhodopsin neurons, which are in communication with and in turn stimulate light-emitting cortical neurons. In one limiting example, emitter devices 1020A, 1020B, 1020C, 1020D may receive and subsequently emit light in a cascading order to form a rastering effect on the cortical neurons. That is, emitter device 1020A may emit light first, followed by emitter device 1020B, then emitter device 1020C, and finally emitter device 1020D before emitter device 1020A emits light again and begins the sequence once again. In other non-limiting examples, emitter devices 1020A-1020D may fire at random based on the desired operation of system 1000, and/or at least two emitter devices 1020A-1020D may fire at a single time or simultaneously.

Emitter device 1020 may be formed as any suitable device, component, and/or feature that may provide the generated light to LGN-Channelrhodopsin neurons to stimulate the corresponding/in communication light-emitting neurons, as discussed herein. In a non-limiting example, emitter device 1020 of emitter assembly 1002 may be formed as a grating emitter. As discussed herein, emitter device 1020 may be formed a single grating emitter or a plurality (e.g., two) stacked emitter devices 1020-1, 1020-2 (see, FIGS. 15C and 15D) to improve emission of the light toward the bioluminescent cortical neurons.

As discussed herein, the light-emitting neurons are in communication with the LGN-Channelrhodopsin neurons that are exposed to the generated light from emitter device 1020. Exposure to the light in LGN-Channelrhodopsin neurons may in turn stimulate and/or illuminate the light-emitting neurons through neural synaptic transmission. In a non-limiting example, the light-emitting neurons internally emit light due to genetically-encoded bioluminescence due to calcium activity. In other non-limiting examples, the light-emitting neurons may include genetically encoded or otherwise dyed neurons, such as those emitting photons due to bioluminescence, fluorescence, and/or phosphorescence calcium and/or voltage signals. Additionally, although discussed and identified throughout as “bioluminescent,” it is understood that systems and/or processes may utilize “light-emitting” neurons as described and defined herein.

System 1000 may also include detector assembly 1004. Detector assembly 1004 may be formed and/or positioned adjacent emitter assembly 1002. Additionally, detector assembly 1004 may be positioned substantially adjacent to the light-emitting cortical neurons being stimulated by light emitted by emitter device 1020. Turning to FIGS. 15C and 15D, with continued reference to FIG. 15A, detector assembly 1004 may include a plurality of semiconductor detector device 1022 (hereafter, “detector devices 1022”). Detector devices 1022 may be positioned adjacent each of the plurality of emitter devices 1020 of emitter assembly 1002, as well as the plurality of simulated light-emitting cortical neurons. As shown in FIG. 15C, a plurality of detector devices 1022A-1, 1022A-2, 1022A-3 may correspond to and/or may be positioned adjacent a single emitter device 1020A-1. In the non-limiting example shown, five distinct detector devices 1022 may be positioned adjacent a single emitter device 1020 of system 1000. However, it is understood that detector assembly 1004 may include more or less detector devices 1022 as shown. Each detector devices 1022 of detector assembly 1004 may detect photons generated by the stimulated light-emitting cortical neurons. That is, upon being exposed to emitted light from emitter device 1020, the stimulated light-emitting cortical neurons may generate photons specific to the associated color with their light properties or characteristics. Detector devices 1022 may be any suitable device, component, and/or assembly that may detect the photons emitted by stimulated light-emitting cortical neurons. Additionally, detector devices 1022 may be in communication with and/or connected to additional components (e.g., CMOS circuits 1032, 1034) that may aid photon sensing, signal amplification, transmission to other circuit elements designed to parse the spatial location of the element and color information involved, compress and encode the data for efficient wireless transmission to another high-speed processor present included in and/or outside of system 1000.

As shown in FIG. 15D, detector assembly 1004 may also include a plurality of optical filtration devices 1030. More specifically, detector assembly 1004 may include a plurality of optical filtration devices 1030 disposed over each of the plurality of detector devices 1022. Each of the plurality of optical filtration devices may allow a distinct, predetermined wavelength of the photons generated by the stimulated light-emitting cortical neurons to pass or be received by the corresponding detector device 1022. For example, five distinct optical filtration devices 1030 may each over and/or be disposed over the five detector devices 1022 positioned adjacent each emitter device 1020. In the non-limiting example, each of the five optical filtration devices 1030 may be tuned to allow a specific wavelength of photons associated with five colors of the light-emitting characteristics (e.g., red, green, yellow, orange, blue) to pass through to detector device 1022. Where “red” photons are generated by the cortical neurons, the associated filtration device 1030 that is tuned to the wavelength of the “red” photons may allow the photons to pass to detector device 1022 and be detected. The remaining filtration devices 1030 may block, scatter, and/or absorb the “red” photons, and only the detector device 1022 disposed below the single filtration device 1030 may detect photons.

Optical filtration devices 1030 may be formed from any suitable device, component, and/or feature that may allow a predetermined wavelength for the photon to pass through. In a non-limiting example, optical filtration devices 1030 may be formed as tuned photonic crystals. The photonic crystals may have a distinct periods of dielectric constants to allow the photon having the corresponding distinct, predetermined wavelength to pass to the corresponding semiconductor detector device 1022, as discussed herein.

As shown in FIGS. 15C and 15D, system 1000 may include additional features formed therein to improve functionality/operation and/or reduce the space/size of system 1000. For example, system 1000 may also include a plurality of through silicon vias 1026 (hereafter, “TSVs 1026”) that may interconnect components or features of system 1000 to other components. For example, TSV 1026 along with interconnects 1028, may operably/electrical couple, and/or place detector device 1022 in communication with CMOS circuits 1032, 1034 that may aid in the detection/processing/transmission of data obtained by detector device 1022.

The capability to interface (in a bi-directional fashion) with several square centimeters of brain surface area (that has been transfected to create, for example, bioluminescence in neurons) may address dysfunction in the ascending pathways of sensory systems, such as those found in age-related diseases humans face. Age-related macular degeneration is one initial and currently understood neural application for system 100—but system 1000 could encompass other neural-based operations including, but not limited to hearing loss, olfactory inputs, and haptics.

It is also possible to visualize applications for system 1000 that are outside of biological systems—due to the intimate integration of emitters and detectors, each operating at multiple, designed, wavelengths may be utilized for topographical mapping of objects at micron scale, high sensitivity sensing and mapping of micron-sized contaminants through optical means (such as fluorescence), enhanced 3D mapping of visual fields, etc.

The emitters and detectors arrays may not be separated from each other. The emitter may need to be in close proximity to the neuron (subtending the appropriate solid angle). The detector group simultaneously may need to be in close proximity to the neurons to ensure photons emitted by the neurons underneath are collected efficiently.

Each element (e.g., emitter, detector) in the array may need to have light power delivered to it, modulated by the element before emitting that light (e.g., MI) towards the brain surface. Each element in the array may include multiple semiconductor detectors, along with CMOS circuit elements designed to sense at the single-photon level, locally amplify the signal and transmit it to other CMOS circuit elements designed to parse the spatial location of the element and color information involved, compress and encode the data for efficient wireless transmission to another high-speed processor present outside the body. Such functionality may be designed by using 3D-integration of two chips connected using ‘through silicon vias’ (TSV) for low-latency information transfer, in addition to electrical bias power for the semiconductor detectors. A small array of unit cells is shown in FIG. 15C. FIG. 15D shows the 3D-integration of chips using TSVs.

Rather than using a conventional large array of light emitting diodes (LED) that are individually controlled, with associated energy inefficiencies associated with stand-by power leakage, system 1000 uses a single laser diode (or a small number of laser diodes). The light from this integrated source may be channeled through single-mode photonic waveguides designed to efficiency transfer light to the desired emitter (or small number of such emitters)—e.g., grating emitter arrays. Also, rather than LED arrays for interfacing with the brain; a “switch matrix” including waveguides and MRRs to progressively switch the light towards the desired emitter may be used in system 1000. 128 emitters, for example, can be individually addressed with just 7 energized MRR-pairs in the ‘switch matrix’ (the rest remain quiescent, needing no power). Tuning power (much lower levels) can be applied to the MRRs to compensate for any fabrication-related variation. The MRRs may couple light efficiently into the waveguide of choice—the tradeoff in terms of lower Q can be accommodate by detuning the opposite MRR and pushing the resonance far enough away.

The emitter is preceded by an MZI that can modulate the light to get the 256 levels of gray. Also, the source diode laser may be modulated to accomplish intensity variation, in the case where only one emitter is active; or use both approaches for energy efficiency.

The tunable MRRs, and MZIs can use thermal or electrooptic methods for modulating the refractive index. Electrooptic methods can use materials like AlN or LiNbO3. Thermal methods can be used for any, including materials like SiN. These are transparent in the visible wavelengths of interest, and can be used for waveguides as well, or a combination of materials can be used (such as SiN for waveguides, and AlN or LiNbO3 or other electrooptic materials for modulation).

The output of the emitter may be designed to have not a Gaussian beam profile, but rather a flat intensity profile across the 250 um×250 um region, at ˜1 cm depth into the cortical tissue. This may be accomplished through, for example, emitter grating design (including spatially distributing sections of the emitter with varying grating pitches and/or breaking it up into sub-sections of emitters that are spatially distinct within the ‘pixel’). System 1000 may include 2 or 3 level gratings so that power is efficiently transferred out of the chip, rather than half the power radiating away from the brain as would be the case for single-layer gratings (e.g., conventional single-layer grating). Use of multiple layers for photonic waveguides also may permit efficient use of available space for elaborate grating emitter design.

Additionally, the detector of system 1000 may use of photonic crystals (either 1D, 2D or multi-layer 2D) fabricated on top of the ‘standard’ semiconductor detector. The photonic crystal is fabricated by a precisely chosen periods of dielectric constant variation that permit light of certain band of wavelengths to propagate through while other wavelengths are subject to destructive interference. The dielectric constant variation can be created by using transparent materials of different refractive index, or by using metal features interspersed in precise fashion with transparent dielectric. 193 nm optical lithography may be used to fabricate these structures.

In other non-limiting examples, system 1000 may include a 128×128 array of emitter-detector elements that fits into a 3D-integrated chip is envisioned. Such a large array, capable of operating at high speeds, and with high sensitivity, may include a photonic integrated circuit to be integrated with electronic circuits.

### EXAMPLES (PROPHETIC)

Embodiments of the present disclosure relate to the development and testing in subjects, such as non-human primates (NHPs), of advanced integrated nanophotonic technology necessary to create cortical neuroprosthetics that will naturalistically stimulate the visual cortex with synaptic precision. In use, the system will restore foveal vision in blind patients. Additional applications will extend to non-visual cortical regions to restore other sensory and cognitive functions. The result will be an innovative streaming video projecting/optical-sensor implant that will stimulate the visual cortex with functional precision at the highest attainable acuity and contrast perception, with full gain control and oculomotor function. The photonics will employ optogenetic neural stimulation from beamforming, coherent light emitter arrays that optimize power calibration (prosthetic contrast gain control) by reading out genetically encoded bioluminescent cortical responses with a sensor made of p-i-n photodiodes for real-time feedback.

Because embodiments of the present disclosure may precisely target the input synapses from the mapped lateral geniculate nucleus (LGN) afferents in the foveal region of vision-adjusting the power by sampling neural responses in a real-time control loop-embodiments will theoretically achieve the highest attainable acuity and contrast sensitivity found in natural vision. Embodiments include testing each iterative stage of device development in subjects such as NHPs to ensure optimized stimulation and read-out from the cortex for rapid translation to clinical use in humans.

Embodiments of the present disclosure create and test nanotechnology to control cortical circuits with a non-percutaneous, fully implantable device. Embodiments include developing components in photonics nanofabrication facilities in stages, including prototyping and benchtop testing of chips, followed by calibration in NHPs. The results will provide feedback for a next round of hardware development in the fabs. Three iterations (3 objectives) will result in scalable neuroprosthetic components configurable for all-optical interrogation in theoretically any cortical map. The inventors will leverage ongoing work in ultra-widefield imaging techniques in NHPs, bioluminescent calcium recordings, and all-optical interrogation methods of the brain, to characterize the newly designed and fabricated integrated photonics devices, to achieve a fully implantable prosthetic with no percutaneous connections. Because more is known about the precise map of LGN ON- and OFF-channel afferents to the primary visual cortex (V1) than for any other cortical region, the first device will be designed to restore vision in the blind. Preliminary data suggest that arrays of emitters/detectors spaced with 250 μm pitch will ensure optimal patterning to target individual LGN input modules into V1 without unwanted targeting of adjacent hypercolumns. This will result in synaptically precise stimulation without spatial gaps in retinotopic coverage that will set the stage for the future development of 40+×40+(1+ cm²) foveal arrays in freely roaming NHPs. The emitter/detector patterning will adjust to other cortical regions once their thalamic input patterning is precisely known.

Embodiments will restore vision in the blind, and provide the infrastructure to develop prosthetics that generalize to other brain areas mediating sensory and cognitive functions.

Initial Development and Testing of Individual Advanced Nanophotonics Devices

Embodiments of the present disclosure include fabricating and testing individual emitter/detector devices. Device embodiments will achieve emission by channeling coherent light from a laser source through optimized waveguides on Si wafer chips, switched on/off with a set of advanced nanophotonic Micro-Ring Resonators (MRRs). Embodiments will electronically control Mach-Zehnder Interferometers (MZIs) positioned along the waveguides to serve as pulse-width modulation (PWM) devices for controlling light level, with temporal precision of >50 kHz, to control the power emission of an attached grating emitter. Detector device embodiments will have five independent p-i-n diodes filtered chromatically with tuned photonic crystals. Following from established ultra-wide field all-optical interrogation techniques, embodiments will include preparing NHPs by transducing optogenes into the LGN, with multicolor bioluminescent proteins transduced into a large >3 cm² field of V1, calibrated against two-photon calcium imaging of jGCaMP7 fluorescence from the same neurons. Embodiments will empirically determine the scatter and penetration depth of coherent light emitter/detector devices in the NHP cortex.

Development of Multi-Element Photonic Arrays and Packaging Techniques for NHP Testing Embodiments of the present disclosure will leverage the results of the objectives above to fabricate separate 4×1 arrays of emitters and detectors. Embodiments include implanting and calibrating the devices in NHPs, and combining them to achieve accurate targeting, high depth penetration, and optimized beamforming to overcome light-scattering in the cortex.

Develop and Test Perceptual Causal Model of Integrated Fully-Scalable Arrays

Embodiments of the present disclosure include integrating and co-packaging emitter/detector arrays from the objectives above as 4×4 integrated photonics chips optimized for cortical all-optical interrogation. In embodiments, multiple chips will be co-implanted in a subject such as NHP V1 to prosthetically stimulate and read-out multiple retinotopic positions for the behavioral characterization of prosthetic vision. These arrays will be scalable to arbitrarily large chips with ultra-large arrays of thousands of emitter/detectors for implantation as non-percutaneous cortical prosthetics, ready for full preclinical testing.

Transformation Significance

The International Agency for the Prevention of Blindness expects 196 million people to suffer from macular degeneration worldwide this year. Millions more suffer from traumatic ocular injury or glaucoma or other foveal defects. The result is foveal blindness⁹ in 3-5% of the global population. Retinal implants can help only a fraction of patients, and no therapy exists to restore foveal vision at the highest attainable acuity.^{1,10} Embodiments of the present disclosure will clinical advance cortical prosthetics by providing an Optogenetic Brain System (OBServ) of the present disclosure as described herein. Embodiments will accomplish synaptically precise optogenetic functional activation of mapped and characterized LGN afferents in non-human primate (NHP) primary visual cortex (V1), with measurements of the cortical responses without a microscope. OBServ will employ innovative nanophotonic optogenetic stimulation, calibrated for NHP cortex. Feedback will derive from a novel hyperspectral imaging system that will record multicolor bioluminescent calcium responses from V1. Embodiments will stimulate LGN boutons in a pattern that mimics naturalistic input. Embodiments will leverage advances in retinal implant technology to optimize our design for contrast sensitivity, acuity, and form vision. When available clinically, embodiments will include,
a. A person with vision loss puts on a specially designed set of glasses. Each lens contains two cameras: one to record visual information in the person's field of vision; the other to track their eye movements.
b. The eyeglass cameras wirelessly stream the visual scene information from the eye-tracked foveas of the patient to two neuroprosthetic devices implanted over V1's foveal region.
c. The neuro-prosthetic devices process and project the visual information onto a specific set of excitatory neurons in the brain's hard-wired visual pathway. These neurons will be genetically encoded with channelrhodopsin proteins, making them surrogate photoreceptor cells, which will function much like those in the eye's retina.
d. The surrogate photoreceptors will be stimulated with light projection devices to relay visual information to the primary visual cortex, initiating the cascade of brain processing that leads to visual perception.
e. The devices will continuously calibrate the visual signals to optimize prosthetic contrast and clarity.

In embodiments, the present disclosure includes the manufacture nanophotonics devices, validated/calibrated in NHPs, to serve as the neuroprosthetic devices. Embodiments include establishing the nanophotonics industry infrastructure to create optogenetic prosthetics for NHP cortical regions, including OBServ in V1. In support of future FDA approvals towards eventual human clinical trials, embodiments will test OBServ's spatial and stereoscopic acuity, contrast sensitivity, and utility for foveal visual stimuli discrimination in NHPs. The use of NHPs at this early stage is critical, as cortical circuits vary widely between taxonomic orders. Thus, rodent testing would not translate to humans, not only due to the lack of a murine fovea, but also because of vast differences in the functional architecture and mapping of the LGN afferents entering V1.

Microstimulation of the visual cortex in blind patients, using electrodes, can produce visual phosphenes, which result from the non-selective nature of activating across diverse neuronal populations in cortical circuits. Retinal implants are helpful and in current clinical use to stimulate the visual pathway but serve only patients with intact retinal ganglion cell layers and healthy optic nerves. In contrast, embodiments of the present disclosure help patients with optic neuropathies. Though previous prosthetic techniques have achieved perceptible discernibility, no systematic methods exist for encoding a wide array of naturalistic stimuli into cortical circuits with high-contrast. Embodiments of the disclosure include leveraging the field's detailed state-of-the-art knowledge of V1 circuits. Current understanding of LGN-to-V1 connectivity in NHPs its specific anatomy and function—is unsurpassed by any other model or circuit in the brain. The fundamental organizing principle of V1 is the hypercolumn, recently redefined in the layer 4 LGN inputs as encompassing one ON and one OFF LGN-input from each eye, encoding each retinotopic position in a mosaic.^{3,4} The location of each V1 neuron within the LGN hypercolumn map determines its orientation selectivity (OS). Embodiments of the present disclosure will directly and precisely stimulate each LGN input module—which contains purely glutamatergic excitatory LGN boutons-avoiding unwanted targeting of either inhibitory cells or nearby untargeted input modules. Embodiments will achieve naturalistic prosthetic function with the synaptic precision of the biological inputs into V1. In embodiment, the methods, compositions and devices will restore foveal vision to as many of the world's blind patients as possible and to create the nanophotonics fabrication infrastructure for prosthetic development in other cortical areas.

Results

The fundamental organizing principle of V1 is the hypercolumn, which is fed by one ON- and one OFF-column input from the LGN^{3,4}, creating a field of homogenous retinotopically overlapped LGN afferents that embodiments will stimulate optimally using a 225-275 μm-pitch array such as a 250 μm-pitch array of nanoscale emitters that leverage nanophotonic devices fabricated using a wafer program.⁸ (See for example, FIG. 1A-1D).

Although discussed herein as having non-limiting examples of pitch spacing (e.g., 225 to 275 μm), the one or more arrays of emitters/detectors may be spaced at a pitch that is at least twice as fine as the pitch of the intrinsic circuits of the neural system. For example, to target individual LGN input modules to V1 in non-human primates, without spilling over to unwanted adjacent hypercolumn targets, a pitch of 225 to 275 μm would be optimally spaced, whereas the optimal pitch may increase in humans, where the LGN input modules projecting into V1 are more widely spaced.

Embodiments include [symbol] long-term single-cell Ca-imaging and optogenetics at columnar scales with 2P and 1P in NHP V1, and described further in a PLoS Biology paper.⁶ (See for example, FIG. 2A-2D).

Recent in vivo 2P microscopic All-Optical Interrogation (AOI) techniques,¹¹⁻²⁵ developed in part by the Macknik/Martinez-Conde labs,^{7,26} will serve as the foundation for the prosthetic approach. See for example, FIGS. 3A-3C.

Cortical NHP CED of AAV virus, targeted with techniques developed by the Macknik/Martinez-Conde labs, will bypass the blood-brain-barrier and produce >90% penetrance in NHP cortex excitatory pyramidal neurons. Data show that expression is enhanced by the Tet-OFF (TREG3) viral expression system, which will be employed to ensure strong expression of bioluminescent and fluorescent reporters in the face of intracellular regulation of mammalian genes.^{6,27,81} Data includes expression of pyramidal neurons in ultra-large FOV 2P images created in awake NHP V1 cortex (See FIGS. 4A-4E).

Embodiments build on previous advances in NHP imaging chamber design, towards long-term patency and brain tissue health with increasingly large windows.^{57,29-34}. Embodiments include imaging implant design that will solve several outstanding challenges to prosthetic design in the brain, which will inform the development of fully implantable devices in this and future studies: 1) difficulty with positioning high-NA objectives near the brain; 2) creating a craniotomy sufficient for ultra-large format imaging (2+ cm diameter) with a flat imaging window against the surface of the brain; 3) adjusting the imaging window to changes in swelling and pressure in the brain, such as those that may occur due to hydration changes and other physiological factors; 4) preventing the growth of dura and biofilms that cloud the imaging window; 5) follow-on MRI imaging of the animal post-implantation. Embodiments achieve these goals with an innovative design that combines the above-described advances with an engineered-silicone support system having the same Young's Modulus as the brain, to optimize both suction- and pressure-regulation of the implant's physical interaction with the brain's surface⁷ (See e.g., FIGS. 5A-5G).

Mapping cortex with sensory driven forward modeling is not possible in cases where sensation is lost, such as in cortex representing a lost limb, or in the case of mapping visual cortex in the blind. Yet to optimize naturalistic perception from prosthetic inputs, matching the artificial stimulation to the existing cortical map is critical.

Embodiments include mapping visual space in the blind.

In embodiments, optimized dynamic spatiotemporal noise optogenetic stimulation is used to find the spatial pattern of stimulation that maximizes the cortical responses. See e.g., FIGS. 6A-6D. In embodiments, the entire region of cortex is first illuminated with a background level of optogenetic stimulation—the optogenetic equivalent of a “gray screen” in vision research. Brief increments and decrements will be presented randomly and independently at each spatial location, while recording bioluminescence responses. The data analysis will determine the spatial pattern of optogenetic stimulation increments and decrements that evoke interact to mutually enhance, interfere, or not affect each other. Central hot spots in this spatial pattern of responses will correspond to the ON and OFF afferent domains (See e.g., FIG. 6A). To disambiguate which of the domains are ON vs OFF, embodiments will rely on factors such as: the cancellation of responses when to interacting domains are co-activated, and the observation that OFF-domains are more numerous than ON-domains.

Once the ON- and OFF-domains have been identified, the fundamental patterning principles of V1 (FIG. 1) will form the basis by which embodiments model the maps that will create in NIHPs using optogenetic techniques.

The basic logic of the optogenetic stimulation schema is illustrated in FIG. 7. Increasing the optogenetic stimulation at a series of adjacent ON-domains (yellow stars) while simultaneously decreasing the optogenetic stimulation at the corresponding off-domains will evoke a percept of an oriented bright line. Reversing the stimulation (increments on OFF-domains and decrements in ON-domains) will evoke a dark bar at the same location and orientation. Shifting the stimulation in the OD columns corresponding to one eye or the other will evoke a stereoscopic percept, corresponding to either crossed or uncrossed retinal disparity.

Embodiments of the present disclosure include the fields of neuroscience, biomedical engineering, materials science, and integrated photonics (design, chip fabrication, and packaging), leveraging combined innovations into a new transformative and disruptive technology to serve as a cortical brain prosthetic. Embodiments of the present disclosure may be referred to as a system the Optogenetic Brain System—or OBServ. Although embodiments have the potential to operate in any cortical area that lies on the surface under bone (where the implant typically mounts), so long as the thalamic input projection map to that cortical region is well understood, embodiments include developing a system in its first use to drive stimulation in the primary visual cortex (V1) from a head-mounted video camera, targeted by eye-tracking. OBServ will activate thalamic (lateral geniculate nucleus; LGN) neuronal afferents entering V1 with synaptic precision, as in natural vision. Embodiments will accomplish this by independently targeting the individual LGN ON- vs. OFF-channel modules entering VI, using advanced beamforming nanophotonics to achieve optogenetic stimulation. This is an important step, because co-activation of unwanted targets in neighboring antagonistic channel modules could inhibit targeted neurons, reducing contrast and resolution in prosthetic perception. Several strategies are under development to improve targeting, but no other extant all-optical methods will generate optimized and naturalistic spatiotemporal cortical stimulation patterns with full feedback gain control. Embodiments of the present disclosure will position, on the cortical surface, innovative nanoscale 630 nm coherent light emitter devices optimized for quantum-efficiency, which will account for light-scattering using beamforming nanotechnology to achieve deep cortical optogenetic stimulation of LGN afferents. Embodiments will create hyperspectral devices to detect responses from an innovative multicolor bioluminescence calcium indicator system, which will genetically encode into V1 neurons, to provide feedback to control prosthetic gain in real-time. The preliminary data show that this system can theoretically restore foveal vision in the blind at the highest attainable visual acuity and contrast sensitivity, to allow future blind patients normal object recognition in the stimulated field-of-view (FOV). Embodiments include providing high-quality foveal restoration based on the experience of macular degeneration patients. They report that small islands of foveal sparing allow high-quality object perception, despite limited FOVs. As such, embodiments of the present disclosure will enhance the patients' life quality by restoring visual perception in the fovea, as well as expand their productive work-life even in fields typically inaccessible to the blind, such as engineering, architecture, or even the visual arts. Embodiments will extend to cover arbitrarily large regions of cortex for optimized ultra-large prosthetic FOVs, thus reaching into peripheral visual regions beyond the fovea. Embodiments will result in fully-characterized and scalable implantable nanophotonic emitter/detector devices that are calibrated and optimized for non-human primate (NHP) cortex, which can then be configured din any arrangement for any cortical region. Embodiments will calibrate the devices at every stage of development in NHP V1 to ensure direct translation to humans, allowing us to follow-up directly in the next project with preclinical trials of fully implantable non-percutaneous implants in freely roaming NHPs. Embodiments will optostimulate targeted LGN input modules within individually identified cortical columns in specific spatiotemporal patterns to mimic natural vision and will allow for conducting module-level causal circuit analyses without directly perturbing V1 circuits. Embodiments will serve as the basis to produce future prosthetics.

Embodiments of the present disclosure include high-precision hyperspectral detectors to image large fields (10,000's) of V1 neurons that are genetically transduced with Aequorin fluorescent proteins (Aeq-FPs) using adeno-associated viruses (AAVs). This bioluminescence recording technology is expected to be potentially disruptive across neuroscience. See e.g., FIG. 8.

In embodiments, fabricating the photonics include a photonic platform having a 65-nm transistor bulk CMOS process technology on a 300-mm diameter wafer, as described in a recent Nature paper.⁸ In embodiments, the integrated high-speed optical transceivers in the platform developed here, which will operate at 630 nm wavelength, will build on the 1550 nm platform. Nanophotonics can operate at fifty gigabits per second in the O-band of the telecom spectrum.^{8,90} In embodiments, the devices of the present disclosure incorporate the finely interlaced coplanar 250 μm pitch arrays of hyperspectral detector-emitter dyads required for this prosthetic application.

Embodiments include advanced packaging techniques to integrate multiple hyperspectral detector array chiplets onto an underlying photonic chip, combined with the demanding electronic, fiber-optic, and free-space optical input/output schema, and other supporting technology necessary to create fully implantable OBServ prosthetic devices.

In embodiments, AAVs are delivered or administered into a subject such as NHP V1 with a breakthrough, wide-dispersion, high-penetrance Convection Enhanced Delivery (CED) infusion technique adapted for NHP cortex.⁵⁸

Light scattering through brain tissue is a primary concern in high-resolution microscopy of neural activity. By implementing the hyperspectral spatiochromatic bioluminescent imaging system, scatter will only minimally affect OBServ's signal. The lensless detectors solely count colored photons and do not form an image of the cell. This neurophotonic strategy barcodes neurons by their color-encoded by a lookup table of cells—in an entirely new paradigm for detecting and decoding brain activity. One of its main advantages is that OBServ will be robust to photons scattered by cortex or biofilms (which grow on implanted devices over time), because scatter decreases transmittance of photons by only ˜3%—and in some cases can enhance light transmittance due to forward scattering effects.⁹¹ By counting photons (robust to scattering media) rather than imaging cells (sensitive to scattering media), embodiments will both enhance the quality of the recording and the longevity of the implanted device.

Embodiments provide spectrally resolved measurements of neural spatiochromatic barcoding with a co-packaged chipset that includes wavelength-tuned detector elements placed in spatial proximity coplanar to the corresponding emitter, patterned at the required 250 μm pitch to optimize spatial and hyperspectral neural decoding.

Embodiments are applicable to energy-efficient stimulation of LGN input modules, with the innovative deployment of integrated photonic components such as micro-ring resonators that minimize quiescent state energy utilization. At the same time, embodiments will use on-chip Mach-Zehnder interferometry as a micron-scale shuttering system to reduce phototoxicity in the brain tissue and to control emission power from grating emitters. Embodiments will use beamforming to minimize supersaturation of the LGN boutons while taking into account light scattering in the intervening cortical tissue. In embodiments, using a suite of nanophotonic components, is more efficient than one using state-of-the-art microLED technology. Nanophotonics are more expansively configurable as arrays of arbitrary shapes than microLEDs are. Nanophotonics are also less expensive, more robust, and more easily integrated into Si-based nanoscale devices at CMOS foundries, since they do not need the more complex III-V fabrication processes required for microLEDs.

In embodiments, a step-by-step approach is provided for developing integrated, scalable, co-packaged, and coplanar arrays of emitters and detectors.

Neurobiological Background

Embodiments include nanoscale devices to optimally stimulate human V1 circuits optogenetically. Old-world NHPs and humans derive from the same parvorder, and they share foveated visual systems (unlike any other mammal). Thus, old-world NHPs are minimally sufficient to developing stimulation techniques optimized for human V1 foveal prosthetics. As such, there is no better circuit, species, or paradigm in which to determine the fundamental mechanisms of human visual prosthetic activation. The input layer (layer 4) of V1 in macaques is moreover the best understood of all cortical input circuit layers, for any specific perceptual or functional skill, in systems neuroscience. No other brain system's topographic thalamic sensory inputs into cortex are mapped with equal clarity⁹². The layer 4 LGN inputs are organized in interdigitated ON/OFF eye-specific modules to connect the LGN to V1.3′4 Understanding the map at this resolution, which is not yet possible for any other neural system, clarifies the prosthetic stimulation strategy (See e.g., FIGS. 9A-9H).

A long-term NHP All-Optical Interrogation (AOI) techniques in V1 for both individual neurons and circuits is available,¹² and achieved high-resolution imaging including individual dendritic input AOI in awake NHPs as part of a multinational effort.^{7,38} By leveraging this technology against the field's new understanding of the LGN inputs into V1's hypercolumns (See FIGS. 10A-10E)^{3,39}, it is established that they can stimulate the LGN boutons entering V1 from the cortical surface to achieve circuit-level activation of V1 circuits at the functional acuity limit of vision.

Visual Processing at the Input to V1.

Because LGN boutons are purely excitatory and organized as a function of retinotopy, ON vs. OFF contrast-sign^{3,4}, and ocular dominance—in individual hypercolumn modules that are 500 μm wide⁹⁵—their activation will result in perception at vision's acuity limit, at any given position and contrast. Natural retinal stimulation thus activates patterned groups of LGN input modules, resulting in visual perception. Patterns of stimulation lead to specific activation of orientation (and other) tuned cortical cells, and to the perception of visual edges. It follows that a prosthetic that uses this encoding will likewise produce the perception of any contour, shape, or form in the world. This is what we aim to create with OBServ.

Encoding Prosthetic Vision

An encoding algorithm of the present disclosure follows from this principle: if one can stimulate the LGN input modules in the same pattern as does natural vision, one will obtain naturalistic prosthetic vision. The logic suggests that if ones optostimulate an entire region of V1 with an even level of activation, one will produce the optogenetic equivalent of a “gray screen” in vision research. If one then judiciously activates pairs of LGN input modules to stimulate edge perception, one will be able to create prosthetic vision of any edge or contour (See FIGS. 9A-9H).

Nanophotonics Background

Embodiments include creating and providing optimized visible-light beamforming emitter devices and hyperspectral detectors optimized for the dual red-shifted 630 nm optogenetic stimulation with hyperspectral Aequorin bioluminescent protein detection.

Photonic Crystal Spectrometer (Hyperspectral Detector)

Referring to FIGS. 11A, photonic crystals (PCs) are ordered nanostructures of at least two different media having different dielectric constants (or refractive indices), arranged in a periodic form (in some cases with multiple, superposed periodicities in x, y and z directions).

Embodiments provide these periodic structures to interact with the electromagnetic light wave to disallow some wavelengths to propagate (a ‘photonic bandgap’ as shown in FIG. 11B or to alter their propagation direction.^{96,97} Embodiments will employ an understanding of EM fields and their interaction with matter, coupled with high-precision numerical calculations, to design the PC elements required for all of the photonic devices in this project. Embodiments will account for sidewall roughness and top surface roughness in the photonic crystal and waveguide elements, and variations (even at <1% levels) in thickness and width of the structures. Embodiments will iteratively design, fabricate, characterize function, refine the models, and incorporate the effects of fabrication-related variability, along with testing fundamentally novel ideas for efficient spectral filtering and beamforming required for the prosthetic device.

Numerical Calculations

Finite-difference time-domain (FDTD) is a numerical analysis technique for modeling electrodynamic behavior in complex geometry by solving 3D Maxwell's equations in time-domain using finite-difference approximation.⁹⁸ It is a powerful method to simulate the interaction of light with nanophotonic structures and predict emission properties, which may be used to explain experimental observations further (See FIGS. 11B-11D).⁹⁹

According to Bloch's theorem, the modes of a periodic structure can be expressed as:

Eₖ(x)=e^{ikx}uₖ(x) (1)

If the periodicity is a, then u(x) is a periodic function with a period a, where u (x+a)=u (x). So, equation (1) becomes: Eₖ(x+a)=e^{ika} Eₖ(x). The photonic band structure calculation involves determining the angular frequencies, ωₙ(k), as a function of wave vector k for all the Bloch modes in each frequency range.

Silicon Nitride (Si₃N₄) waveguides

Embodiments design and provide single-mode Si₃N₄ waveguides to efficiently route visible light (630 nm wavelength) from a single-mode optic fiber edge-coupled to the chip to the various photonic devices on the Si chips. The Si₃N₄ waveguides will be fabricated on top of a thick oxide layer to reduce loss associated with proximity to the silicon substrate. There will be a thick oxide cladding overlaying the waveguide as well. Si₃N₄ waveguides have low propagation losses in the range of 0.3-1 dB/cm.¹⁰⁰

Grating Emitters

Embodiments provide one-dimensional (1D) PC structures with finite thicknesses of 70-250 nm as 1D grating out-couplers (grating emitters).⁴⁶⁻⁴⁸ In embodiments, they will serve to redirect light propagation from within the waveguides into a new direction perpendicular to the chip surface, towards the cortex. These out-of-plane coupling devices are compatible with high-volume fabrication and packaging processes and allow for on-wafer access to any part of the optical circuit. Using robust and scalable fabrication strategies developed in Galis's lab,¹⁰⁴ in combination with the state-of-the-art capabilities at fabs, the inventors have established the modeling foundations for PC engineering and demonstrated high accuracy placement of fab-compatible PCs and grating structures.^{99,104} The inventors will simulate, characterize, and fabricate PC beamforming design variants that achieve optimal intensity, shape, and size profiles deep within the cortex without lens elements.

Micro-Ring Resonators (MRRs)

MRRs are devices that modulate optical power transmitted along a nearby waveguide at specific wavelengths, determined by the resonant frequencies of the MRR. In embodiments, pairs of Si₃N₄-based MRRs are placed on either side of an ‘upstream’ waveguide to switch optical power into the desired ‘downstream’ waveguides. Switching utilizes a change in the refractive index of Si₃N₄ via the thermo-optic effect, to move the resonant frequency of the MRR away from 630 nm. When one energizes one of a pair of MRRs, one will inhibit the transfer of optical power into that side while permitting the transfer of power through the opposite MRR and into a downstream waveguide. One will employ these as a cascade of shutters to direct light down specific waveguides to specific photonics devices at the correct time. One will cast MRRs in the ‘normally-on’ state (they will transfer light unless power is applied to block the transfer), to optimize power load for the MRR cascades. This will enhance efficiency in the very large arrays we envision for OBServ in the future.

Mach-Zehnder Interferometers (MZIs)

These devices use thermo-optic modulation to vary the refractive index of one of the two arms in the split waveguide, allowing for the coherent light traveling down the two loops to sum constructively or interfere deconstructively. They will serve as shutters and will be designed and fabricated for operation at 630 nm in the “normally-off” state. The MZIs will not only block unnecessary light from entering the brain, but they will also provide pulse-width modulation (PWM), at 50+ kHz frequencies, to each grating emitter, to control optogenetic activation strength of the target synaptic boutons in the brain.

Interface Electronics

The nanophotonics devices require interfaces to outside signal processing and control electronics. The MRR and MZI components are controlled thermally via a thin film metal heater. The amount of power necessary to accomplish the thermo-optic effect in each photonics device varies with the tolerance of each component. We will gate power with a low-side MOSFET switch. One will source the gated control signal from a pulse width modulation (PWM) circuit that is itself controlled by the system microcontroller. The duty cycle of the PWM will determine the optical power delivered, controlled at temporal frequencies exceeding 50 kHz.¹⁰⁵ One will use the p-i-n photodiodes in photovoltaic mode, which realizes the lowest dark current of available topologies. One will gate each diode's cathode line with a MOSFET switch routed to a transimpedance amplifier with its gain set to accommodate the different spectral responses of the p-i-n diodes. The amplifier output (a voltage) will be sampled and converted to a digital value several times over a given time window using a high-speed analog-to-digital converter (at least 10 bits resolution). These multiple samples will be summed and averaged to improve the signal to noise ratio. The final value can be stored in a set of memory arrays on the microcontroller or transferred by USB for off-system processing, with the eventual use of wireless communications planned. Embodiments will design all interface electronics with future miniaturization as implantable wireless ASICs in mind.

Experimental Plan

Embodiments may start with LGN cells, which will be virally transduced with channelrhodopsins, expressed within the LGN boutons lying within the input layers of V1. When stimulated with the correct spatiotemporal waveform of light activation, the boutons will activate V1 neurons directly, just as in natural visual input. Each LGN input module within a given ON or OFF hypercolumn subregion is both large (about 500 μm in diameter), and homogeneous in its receptivefield (RF) characteristics.⁸ It also shares its retinotopy with the other modules in the same hypercolumn. One can theoretically achieve perception at the highest attainable acuity by stimulating an individual LGN input module. Undesired co-activation of neighboring modules or inhibitory cells can result in perceptual glare or decreased contrast. Targeting LGN boutons thus results in synaptically precise retinotopic activation of V1.

It follows that an optogenetic prosthetic that strongly excites a few LGN afferents within a single input module, or weakly excites all of the afferents within the module, will produce an equivalent perceptual result: a spot of perceived light (for ON-channel stimulation) or darkness (for OFF-channel stimulation) at the highest attainable acuity, encoded within the smallest point in the retinotopic map⁸. A pattern of activation across the cortical map, derived by input from a video camera (and modified to the spatiotemporal map of LGN inputs to V1), will serve to restore vision—as it is equivalent to how natural vision functions when the observer views a video screen. Thus, if one stimulates all regions of cortex evenly, except for increased activation of a single ON input module and an OFF module in a neighboring retinotopic hypercolumn, one will trigger the perception of a gray field with two adjacent spots (one white and one black; (See FIG. 8E). All visual stimuli in the world-every oriented edge, surface, and form—is built from these fundamental pointillist building blocks. Embodiments fabricate the components necessary to build an implantable device that will stimulate spots of 500 μm diameter (or smaller) in layer 4, targeted in real-time by a scene camera, taking into account eye position within that visual scene in real-time (just as the natural visual system does). Because all prosthetic devices will fail to attain truly naturalistic stimulation unless they account for rapidly changing visual and cortical conditions, embodiments will integrate into the system a power feedback system that will continually assess and modify stimulation levels, just as the natural visual system does with its intrinsic contrast gain control circuits. Embodiments will monitor V1 calcium activity with an innovative lensless spatiochromatic recording system that measures single-unit calcium activity continually as a function of the arrival of hyperspectral color-coded streams of photons emitted by multicolored bioluminescent proteins (which will transduce into 90% of the pyramidal cells in V1). This will determine emitter transmittance efficacy.

Embodiments will iteratively design, fabricate and calibrate individual emitter/detector devices. Embodiments will produce individual devices. Embodiments will produce linear arrays of these devices and address the integration of multiple nanophotonics devices with control electronics on a single chip. Embodiments will produce scalable 2D arrays (4×4) of the emitter/detector dyads at the proper pitch (250 μm) for optimal OBServ function within NHP V1. Throughout all objectives, one will calibrate the devices in the NHP cortex to ensure optimized function in humans, and we will develop the interface hardware and software to collect and analyze the neural data in real-time, to implement prosthetic gain control in each iteratively more advanced chip. The result will be technology ready for scaling to 1 cm²⁺ arrays for preclinical testing as ultra-large FOV non-percutaneous prosthetic implants.

Development and Testing of Individual Advanced Nanophotonics

Embodiments include preparing two naive NHPs by surgically implanting them with the V1 imaging chambers designed for these experiments. One will scan NHPs with both CT & MRI to target the LGN and foveal V1 for injections and chamber implantation (See FIG. 5). Once prepared, the CED injections and chamber implantations will employ advanced neurosurgical navigation systems and advanced electrosurgical and piezosurgical techniques to maximize accuracy and minimize blood loss and risk of infection. One will design AAVs using the Yamamori lab's bacterial TREG3 promoter, which in NHPs enhances pyramidal cell-targeted gene expression, instead of the standard CamKIIa-promoter.⁵¹ The TREG3 promoter system has the advantage that expression can be up- or down-regulated with orally administered doxycycline as needed.

With this targeting and expression scheme, which the inventors have successfully used in our preliminary data (See FIG. 4), one will inject the LGN with AAVs delivering channelrhodopsins, and V1 with mixed AAVs carrying the Aeq-FPs array of bioluminescent genes, as well as jGCaMP7, as described below.

Injection Details

Embodiments will use CED (FIG. 5) in V1 to deploy a battery of AAVs carrying bioluminescent and fluorescent calcium reporters that target the TREG3 promoter. To achieve a bioluminescent Brainbow (Tet-Bow) calcium reporting in our cortical pyramidal cells, we will inject five different colored Aequorin transgenes, which will intermix stochastically (blue, cyan, green, yellow, and red): AAV5.TRE3G.mTAGBfp2-AEQ.WPRE.RGB, AAV5.TRE3G.mCerulean-AEQ.WPRE.RGB, AAV5.TRE3G.eGFP-AEQ.WPRE.RGB, AAV5.TRE3G.SYFP2-AEQ.WPRE.RGB, and AAV5.TRE3G.Requorin.WPRE.RGB. To benchmark the bioluminescence calcium imaging against a known calcium imaging technology, one will co-inject AAV5.TREG3.jGCaMP7f.WPRE in high-titer. To target this cocktail of AAVs to cortical pyramidal cells, one will include an AAV (AAV5.CamKII.TetOffAdv2.WPRE.RGB) that expresses theTREG3 bacterial promoter selectively within pyramidal cells. One will then compare the hyperspectral bioluminescent Ca+ responses to the two-photon (2P) imaged fluorescent GCaMP7 Ca+ responses from the same cells (See FIG. 4).

One potential concern is that the green-tagged Aeq-FP overlaps in wavelength with GCaMP7: the two Ca+ reporters cannot report Ca+ level at the same time. However, GCaMP7 will only fluoresce in the presence of the excitation light source, which Aeq-FPs does not require, whereas Aeq-FPs only report Ca+ level in the presence of coelenterazine (CTZ), a luciferin molecule necessary to achieve bioluminescence. One will moreover image GCaMP7 with a scanning laser microscope, whereas one will achieve spatial imaging of Aeq-FPs with an array of detectors (i.e., a camera). Thus, one will use 2P to image GCaMP7 responses in the absence of CTZ, and one will image bioluminescence with a hyperspectral array (i.e. OBServ's detectors function as an optimized cortical camera) in the absence of an excitation light source, to minimize crosstalk between the Ca+ reporting systems. If this poses problems, one will prepare a new NHP using four colors of Aeq-FP Ca+ indicators (no green).

Preliminary Results

Embodiments have successfully used the CED technique⁸⁵ to transduce a 3.14 cm² imaging window evenly using just four 40 μL Tet-Bow AAV injections to obtain high gene expression intermixed with GCamp6 (FIG. 4). Embodiments achieved ˜5000 distinguishable colors of Aeq-FP mixing, out of the ˜61,000 neurons that we labeled and individually imaged with 2P (FIGS. 12A and 12B) These findings indicate that each distinguishable color is expressed in ˜12 neurons throughout the entire imaging window. Embodiments imaged z-stacks between the surface and ˜0.5 mm deep, resulting in an imaging volume of ˜157 mm³. Thus ˜1 neuron of each distinguishable color was produced in each 13 mm³ volume (equivalent to 26 mm² of the cortical surface area). Because our emitter/detector array will have a pitch of approximately 250 μm, a square 1 cm² (100 mm²) prosthetic chip will contain 40×40 (1600 total) pixels and ≤four neurons will emit the same color (assuming the same injection results as in our preliminary data). It follows that the probability of any specific color falling beneath any given detector is ≤0.025, and that the probability that two cells with the same color will lie beneath the same detector is ≤0.000625. Scatter from neighboring regions will not be a significant concern: we have modeled light dispersion through 1 mm of cortical media as <100 μm (See FIG. 10), which is less than the pitch between detectors in OBServ. In the event that one has underestimated cortical light scatter and two cells emitting the same color lie equidistantly between four detectors, the discrimination error rate would increase by 4× (to ≤0.0025). This worst-case scenario would nevertheless be ˜20× better performance in single-unit identification than state-of-the-art high-density electrode arrays of 384 electrodes,⁵² which perform spike sorting identifications with an error rate of ˜0.05.⁵³ Thus, the system will theoretically produce errors 20-1000 times less than state-of-the-art neuronal recording systems. It is acknowledge that the two types of recording systems operate at vastly different time scales, and thus we do not suggest that Tet-Bow spatiochromatic imaging should replace electrophysiology in all circumstances. Having said this, embodiments may be ideal for its proposed use in OBServ.

Embodiments will inject the LGN with AAVs delivering red-shifted Chrimson optogenes, using CED to fill the LGN with a single 200 μL injection (AAV2.Thy1S.ReaChR.EQFP670.WPRE). Previous studies have shown that AAV 2.2 is optimal for thalamic cell transduction.³⁴ The preliminary data shows that this technique results in LGN boutons in the cortex that are excitable with light emitted from outside the cortex, without puncturing the pia mater (See FIG. 10). This optogenetic excitation is evidence that LGN boutons transduced with ChR2 can be stimulated from the cortical surface by an 11 mW/mm² light source to drive V1 neural responses.

Design, Fabricate, and Characterize Efficient Photonic Elements for Packaged Emitter Arrays

Embodiments may be fabricated at SUNY Polytechnic Institute's Albany, NY NanoTech Complex state-of-the-art facilities. The 1.6 million square foot complex houses the most advanced public entity owned 300 mm wafer facilities in the world, including over 125,000 square feet of Class 1 capable cleanrooms. The complex includes a 300 mm leading-edge, industrially relevant, CMOS fabrication line.^{109,110} Co-I Galis's lab also houses several deposition systems and a multipurpose state-of-the-art single-photon microscope. We have full access to AIM Photonics' Test, Assembly and Packaging (TAP) facility at Rochester, NY, which provides the capability for nanoscale electronic and photonic packaging technology development, as well as the production capability in the wafer scale, chip-scale and I/O attach processes required by our project.

Develop Photonic Elements Operating at 630 nm

Utilizing the methodology (numerical calculations—nanofabrication) near-infrared emission engineering in PCs has been demonstrated,⁹⁹ one first need to identify the proper design/geometry of the grating emitter tailored to the operating wavelength of interest (630 nm). Embodiments will employ FDTD computations (Lumerical software) as a preliminary foundation to study the effects of structure designs on extraction efficiency, polarization, and directionality and radiation angular distribution to achieve optimum beam intensity profiles for efficient illumination of the desired region of the visual cortex. Embodiments will fabricate corresponding prototypical structures designed for optimal performance at 630 nm.

Comparative steady-state, transient (time-domain), and polarization emission measurements for our numerical benchmarks, will characterize the emission properties of the resulting structures. Electron-beam lithography will be used for rapid prototyping and to minimize expenses. By energy-efficient, we mean rastering light rapidly across the array of desired grating emitters, at ˜60 Hz or faster. MRRs designed to be ‘normally-on’ (and hence transfer light into the neighboring waveguide without additional power supplied) will minimize power requirements, which will be a significant factor in the ultra-large arrays we intend to deploy in the future within prosthetic implants. During the scan, when we energize the selected MRRs, the resonant frequency of the ring will be altered (through thermo-optic induced modulation of the refractive index) to prevent light from propagating through the MRR into the adjacent waveguide. MZIs, designed to be normally-off (to reduce phototoxicity in the brain), will also be designed and fabricated for operation at 630 nm. The MZIs will not only block unnecessary light input on the brain, but also provide pulse-width modulation at low frequencies for individually addressable grating emitters, to determine the optogenetic activation level in each location of the tissue.

Preliminary Results

The Galis lab has recently reported a novel fab-compatible nanofabrication in Nanophotonics^{99,104} for defect-free grating arrays, which enhance 1540 nm-telecom photon flux collection and control (FIG. 11E). This approach overcomes obstacles faced by top-down and bottom-up approaches, which typically result in high surface defect density states in the dry-etch step, or random orientation and non-specific positioning, or require transfer to another substrate and subsequent fabrication steps, thus limiting scalability. AIM Photonics has fabricated MRRs at the C-band wavelengths utilized for fiberoptic telecommunications and will use numerical simulations to guide the team towards optimal designs operating at 630 nm.

Develop Photonic Packaging for the Grating-Emitter Chip at 630 nm

Embodiments provide photonic assembly (coupling of photonic waveguides to optical fibers) required for external optical connectivity (laser sources) to demonstrate that the grating emitter circuit maintains high efficiency. Embodiments will employ a specialized automated optical fiber coupling tool, capable of accurately aligning and attaching optical fibers to maximize the light transmission into the grating emitter chip.

High precision optical fiber coupling is obtainable using a robotic gripper to position optical fiber into a location at the edge of a photonic chip (FIG. 12C). Software controls a laser to light the fiber, positions the fiber at the edge of the chip, and ultimately couples light into the waveguide on the chip (indicated by the red line at the end of the fiber in FIG. 12C (bottom).

Design, Fabricate and Characterize Detectors for Efficiency and Speed at Visible Wavelengths

Several design choices will be explored for the detectors while leveraging the 300 mm wafer fab. Due to germanium's small bandgap of 0.67 eV versus silicon's silicon bandgap of 1.1 eV, embodiments will prioritize Ge designs that utilize extremely short absorption lengths at visible wavelengths. Whereas Si-based detectors experience fewer surface-recombination-related losses than Ge-based detectors, they lose photons that traverse the Si-on-insulator layer, absorbed by the silicon substrate beneath the buried oxide layer. Detectors embodiments will be optimized to the Aeq-FP emission bands and characterized using spectrally filtered light for quantum efficiency, noise, and speed, as well as for sensitivity to any unexpected interference between design parameters occurring in the initial stages of the project.

Develop Photonic Crystal Structures to Function as Color Filters and Characterize their Spectral Efficiency Using Detectors

Embodiment device design considers practical conditions so that the simulation parameters are well within reach of our fab's capabilities. One will demonstrate spatially resolved and wavelength-distinguished coupling of light using photonic crystal arrays. One will leverage FDTD calculations and precisely fabricate optimal two-dimensional (2D) PC geometries for enhanced color filtering. One will experimentally test (light intensity mapping, polarization, photon statistics), using our single-photon microscope system (FIG. 12C), and benchmark the fabricated PC nanostructures at different wavelengths and excitation power densities to refine and improve our understanding by iterative optimization. After chip-level characterization of the detectors with integrated PCs, we will develop packaging techniques to interfacing with external electronics. The high input/output count of the detectors (multiple colors within each array-element) needs careful attention to routing to bondpads, as does the incorporation of a suitable connector that does not interfere with the open optical access to each detector element, minimizing the stand-off distance between the detector and the visual cortex.

Simulations on 1D PC nanostructures for modeling telecom C-band emission have been performed and the results correlated with emission measurements on identical fabricated nanodevices.⁹⁹ The PC nanostructures exhibited a photonic bandgap in the telecom C-band as predicted.

Calibrate Bioluminescence Detection

Embodiments include mapping of V1 with 2P GCaMP7 recordings, followed by mapping with a color camera that records the 1P bioluminescence responses.

Embodiments include conducing hyperspectral 2P z-stacks of the V1 cells of each prepared NHP to establish the bioluminescent colors of each of the neurons one will subsequently map with jGCaMP7 (FIG. 12B). Embodiments including building a color lookup table from this data (See FIG. 8A, 12A, 12C), which one will further label with individual cell-characterized RF data as one conducts the 2P retinotopy, ON/OFF, and orientation mapping (See FIG. 2).

Embodiments will simulate how these imaged stacks may appear to a color camera by building blurred maximum view images from the 3D stacks (as in FIG. 8B). This will estimate the color separation one can expect from our nanophotonic hyperspectral detector technology. Since bioluminescent reporters depend on CTZ delivery, embodiments will apply CTZ (250 μg/200 μL Nanolight CTZ-SOL injected into the CSF in each recording session) through an injection tube built into our custom cortical implant system. Embodiments will compare the Aeq-FP activity against Ca+ imaging in the same neurons to benchmark Aeq-FP emission strength and response speed.

Develop Multi-Element Arrays and Associated Packaging Techniques for NHP Testing

Develop Packaging Processes for 4-Element Dyad Detector-Emitter Arrays

From the foundation established above, embodiments including making 4×1 integrated detector-emitter arrays. This will involve the dicing of 4-element long and narrow hyperspectral detector chiplets from a 300 mm wafer, followed by careful placement (and bonding) of these chiplets onto the surface of individual 4-emitter chips, so that the detectors and emitters form a row of four emitter-detector dyads having 250 μm spacing (FIG. 13A). This integration of detector chiplets on emitter chips will require high positional accuracy to ensure that the emitter is not occluded, with the detectors in the closest allowable proximity to the corresponding emitter. Each array element dyad will comprise an emitter (a Grating Emitter which will be ‘fed’ 630 nm light pulses from the cascade of MRR switches that raster the light across the 4-emitter array, with PWM controlled by an MZI at each emitter on the chip) paired to a hyperspectral detector with five integrated detector devices that are surmounted by 2D photonic crystals designed to select the five bioluminescent peak wavelengths (indicated by colors in FIG. 13A). The electrical connections from the detector arrays will be made by wire-bonding from the detector chiplet surface down to the emitter chip, and thence to bondpads leading to the underlying printed circuit board.

NHP V1 Causal Testing of 4-Element Dyad Detector-Emitter Arrays

In embodiments, NHP's eye movements will be tracked as they fixate a point in return for a juice reward every 2-4 seconds (randomly varying). Embodiments, will simultaneously map V1 using visual stimuli including sparse noise maps and a battery of oriented gratings that tile both orientation and spatial frequency space, taking into account even small microsaccadic eye position changes.^{26,38,56,57} This will generate preference and selectivity maps for orientation, as well as maps of retinotopy, ON/OFF columns, and SF, using 2P GCaMP7 Ca+ activity (FIGS. 2 and 3). Using these maps, embodiments will identify an area near the center of the NHP's imaging chamber that has several adjacent well-characterized hypercolumns with identified ON/OFF columns, and a precisely localized retinotopic position. Embodiments will flash a white/black visual sparse noise spots at the acuity limit of vision, in the regions of the hypercolumns of interest, using randomly changing stimulus contrasts from 0-100% (5 levels of stimulus contrast 0, 25%, 50%, 75%, 100% (FIG. 13B) at 10 Hz. This will identify the specific V1 cells that respond to visual activation of each hypercolumn, and their ON/OFF module positions in space, and it will supply a neurometric curve of calcium response (ΔF/F) for each cell, as a function of stimulus contrast. This is our visual model of stimulus contrast for these cells. Embodiments will then use solely optogenetic stimulation of the LGN inputs, iteratively changing the retinotopic position, size, and brightness of the spots while determining the prosthetically driven neurometric curves of the calcium response (ΔF/F), in the same visually characterized cells, as a function of stimulus power. These neurometric results will be correlated to the visual model results to determine a causal model of prosthetic-equivalent stimulus contrast. Embodiments will confirm the causal model by stimulating individual ON/OFF modules at the correct stimulus strength and pattern to evoke responses within specific predicted orientation-tuned cells (FIG. 13C).

Embodiments will use visual and prosthetically modeled cells to calibrate the bioluminescent calcium reporting system as a full causal neurometric model of visual/prosthetic stimulus-response. Embodiments will determine the hyperspectral color of each cell using our hyperspectral 2P microscope (FIGS. 1.1 & 1.2). Embodiments will then inject CTZ and turn off our fluorescence excitation laser while stimulating optogenetically in the same patterns used to create the previous causal model; this will once again activate the specific orientation-tuned V1 cells, but now one will record their bioluminescent responses with the hyperspectral detector channels of our 2P microscope, instead of with fluorescence imaging of GCaMP7. Embodiments will compare the neurometric curves of the color-barcoded bioluminescent responses to the spatiochromatic color lookup table from the 2P Ca+ imaging of the same cells.

While conducting the experiments above, the Carter lab will build the necessary interface electronics to operate and record from the OBServ 4×1 emitter-detector chips, keeping in mind scalability to future arbitrarily large arrays of emitter-detector dyads.

With a complete model of the V1 bioluminescence response from our characterized cells in hand, embodiments will place the 4×1 OBServ linear array over the specific V1 columns containing those cells. Embodiments will stimulate the cortex from the emitters using the same stimulation strengths used for the previous neurometric curves (using the same laser, now connected by fiber to the OBServ chip). Embodiments will use OBServ's hyperspectral detectors to measure the bioluminescence responses to the stimulation, to create neurometric curves and full causal calibration of the emitter-detector technology. This will provide feedback for adjustments to the chips' beamforming, sensitivity, and hyperspectral bandgap structure, until the benchmarks of OBServ and the 2P recordings are mutually consistent.

Develop and Test Perceptual Causal Model of Integrated Fully-Scalable Observ Arrays

Develop Packaging Processes for 4×4 Integrated Detector-Emitter Array

After adjusting the hardware using the calibration results, embodiments will provide 4×4 integrated emitter-detector arrays (FIG. 13A). These arrays will be spaced at 250 um pitch in X/Y directions and packaged for further testing. Embodiments include a design of each emitter to illuminate a 250 μm spot ˜1 mm deep within the cortex, as one cannot optimize the alignment of any given emitter to the underlying hypercolumns. Given the functional architecture of the VI hypercolumn maps, a 250 μm pitch will ensure that at least one in every four emitters tiles the hypercolumn map optimally to activate every single ON/OFF module beneath the chip, achieving precise targeting of every module without unwanted leaking into neighboring modules. Embodiments will determine emitter alignment empirically post-implantation, and inactivate poorly aligned emitters.

Behavioral Calibration

Embodiments include calibrating OBServ by optimizing the error function derived by perceptual responses obtained from both real vision and optostim. Embodiments assess the quality of the optogenetically stimulated perception using the same calibration paradigm, by measuring the prosthetically evoked perception-assessed behaviorally-compared to visual stimulation.

Embodiments will train NHPs to perform a 2-alternative-forced-choice contrast-sign discrimination task. NHPs will fixate a cross on a monitor with a 50% gray background for 300 ms. (FIG. 14A). Two small abutting spots-one dark and one light compared to the gray background-will be displayed at random foveal positions near the fixation point at for 120 msec. The NHP will discriminate the position of the brighter stimulus by making a saccade to the closest of two checkered circles that appear after the spot stimuli turn off. Correct discrimination will result in a juice reward. On half of the trials, one will replace visual stimulation with prosthetic stimulation (using either a 1P laser spot delivered via our 2P microscope or with OBServ). Embodiments will adjust the optical stimulation parameters (spot strength, duration, and in the 2P scope embodiments will also vary spot size) until the NHP's performance with prosthetic stimuli matches visual performance. Embodiments will simultaneously record the Ca+ responses from the visual stimulation trials with either 2P or the 4×4 OBServ arrays (using bioluminescence).

Analysis: Embodiments will calibrate the causal neurometric curves with the psychometric curves. Embodiments will compare the neural responses from visual and optogenetic stimuli (from both the 2P microscope and OBServ) with neurometric/psychometric curve calibration analyses (FIG. 14A)¹¹¹; this will determine whether prosthetic vs. visual contrast responses are perceptually comparable FIG. 14A. These experiments will reveal either a linear or a nonlinear relationship between GCaMP7 and bioluminescence responses compared to visual stimulation as a function of perception. Any enhancement we find in the 2P microscope over OBServ's stimulation will be characterized and sent to Poly for future modifications to the grating emitters in OBServ's chip.

Lessons learned and application to future scaling to full systems-: Integration of the detectors and the MRR/MZI/Emitter system will be designed for scalability for array sizes of up to 128×128, to interact with 1 cm² or more of V1's surface. This will enhance the FOV into the visual periphery. Embodiments will provide a fully implantable OBServ chip having communications and power ASICs that support operation non-percutaneously. Embodiments will perceptually test these larger arrays at the level of object recognition, and optimize the real-time gain-control system.

Vertebrate Animals

Animals Three adult male and female Macaca mulatta will be purchased and maintained. Rhesus macaques, because of the long history of their use in behavioral neurophysiology, because they provide the closest link to humans of the available models of the visual and oculomotor systems, because they are amenable to cognitive and perceptual training, and because of our experience with this species. Following the principle of the 3 Rs, we have REDUCED the number of animals to the minimum necessary to verify the results. The animals are housed individually in NHP cages (dimensions 89 cm width, 147 cm height, 125 cm depth, including perch) for the duration of the experiment. Monkeys are provided with environmental enrichment, including a television, fruits and vegetables, food puzzles, perches, Kong toys, mirrors, and other enrichment tools as available, along with visual and auditory contact with several other monkeys that are also housed individually in the same room, and positive daily human contact. The room has a 12-hour light/dark cycle. Regular veterinary care and monitoring, balanced nutrition, and sensory and social environmental enrichment are provided in accordance to the National Institutes of Health Guide for the Care and Use of Laboratory Animals for animal experiments, to maximize physical and psychological well-being. Monkeys have abundant access to food (i.e. feed biscuits are provided twice a day (approximately 12 biscuits/monkey), Purina Lab Diet Monkey Diet, Product #0001333).

Implantation Surgery Surgeries will be carried out under the guidelines of the Institutional Animal Care and Use Committee (IACUC) at DHSU, under the supervision of the attending veterinarian. A structural MRI of the monkeys' brains will be conducted before placement of surgical implants. Food and water will be withheld the night before the surgery, and antibiotics will be given prophylactically (Timentin, IM, 50 mg/kg; Gentocin, IM, 1.5 mg/kg). Anesthesia will be induced with ketamine (10 mg/kg IM), the head will be shaved and surgically prepped, and loosely supported with towels. The larynx will be sprayed with a local anesthetic (Lidocaine), intubated with a tracheal tube for the duration of the surgery and administered atropine sulfate (IM, 0.04 mg/kg) to reduce secretions; a venous cannula will be inserted and gas inhalation anesthesia (0.5-1.5% Isoflurane) will be administered. Respiration, pulse rate, SpO2, ETCO2 levels and temperature will be continuously monitored and recorded with the values of the anesthesia infusion rate and physiological monitoring (electrocardiogram, heart rate, oximetry, pupil size, withdrawal reflex, corneal reflex). The animals will be implanted with a head post and bilateral recording chambers positioned over V1. Analgesics (buprenorphine, IM, 0.005 mg/kg) and antibiotics (Timentin, IM, 50 mg/kg; Gentocin, IM, 1.5 mg/kg) will be administered post-op. We will occasionally perform minor surgical procedures to maintain the interior of the recording chambers in good condition. These minor surgeries occur rarely and usually take half an hour or less, under ketamine anesthesia (10 mg/kg, IM).

Antisepsis precautions To create a craniotomy within the recording chamber, once the monkey has fully recovered from the implantation surgery, we will first anesthetize the animal with ketamine (i.m., 10 mg/kg plus atropine 0.04 mg./kg.). We will use a trephine to create a craniotomy at the bottom of the chamber. We will conduct a durotomy implant the custom 3D-printed PEEK hermetically-sealed pressure and suction regulating chamber system developed by the Macknik lab (FIG. 5). We will clean the chamber daily with sterile saline and betadine solution before and after each imaging session, injecting prophylactic antibiotics into the CSF through the CED tubes of the chamber after each session. We will routinely disinfect the guide tube and the electrode with Nolvasan before each recording.

General recording procedures Eye-position measurements are standard (SMI XView NHP binocular video eye-tracking). The system integrates with the Avotec visual stimulation system, and can be used in a standard monkey behavioral chamber (Crist Instruments, Inc.). We will conduct CED injections and 2P imaging following the procedures (FIG. 4).

Liquid intake control The monkeys will perform perceptual and oculomotor tasks to earn liquid rewards, and their fluid intake will be controlled, monitored, and logged daily. The animals' weight will also be monitored and kept at >90% of pre-training weight. Monkeys typically earn over 80% of their daily fluid allotment during the testing sessions, and receive water and/or fruit supplements after the experiments.

All patents, patent publications, and references are herein incorporated by reference in their entireties.

References incorporated herein by reference include the references of Table C

TABLE C

| | |
|---|---|
| 1 | Macknik, S. L., Martinez-Conde, S. & Haglund, M. M. The role of spatiotemporal |
| | edges in visibility and visual masking. Proc Natl Acad Sci U S A 97, 7556-7560, |
| | doi: 10.1073/pnas.110142097 (2000). |
| 2 | Macknik, S. L. & Haglund, M. M. Optical images of visible and invisible percepts in |
| | the primary visual cortex of primates. Proc Natl Acad Sci U S A 96, 15208-15210 (1999). |
| 3 | Kremkow, J., Jin, J., Wang, Y. & Alonso, J. M. Principles underlying sensory map |
| | topography in primary visual cortex. Nature 533, 52-57, doi: 10.1038/nature17936 (2016). |
| 4 | Lee, K.-S., Huang, X. & Fitzpatrick, D. Topology of ON and OFF inputs in visual |
| | cortex enables an invariant columnar architecture. Nature 533, 90-94, |
| | doi: 10.1038/nature17941 (2016). |
| 5 | Acker, L., Pino, E. N., Boyden, E. S. & Desimone, R. FEF inactivation with improved optogenetic |
| | methods. Proceedings of the National Academy of Sciences 113, E7297-E7306 (2016). |
| 6 | Ju, N., Jiang, R., Macknik, S. L., Martinez-Conde, S. & Tang, S. Long-term all-optical |
| | interrogation of cortical neurons in awake-behaving nonhuman primates. PLOS Biology |
| | 16, e2005839, doi: 10.1371/journal.pbio.2005839 (2018). |
| 7 | Macknik, S. L. et al. Advanced Circuit and Cellular Imaging Methods in Nonhuman |
| | Primates. The Journal of Neuroscience 39, 8267-8274, doi: 10.1523/jneurosci.1168- |
| | 19.2019 (2019). |
| 8 | Atabaki, A. H. et al. Integrating photonics with silicon nanoelectronics for the next |
| | generation of systems on a chip. Nature 556, 349-354, doi: 10.1038/s41586-018-0028-z (2018). |
| 9 | Pascolini, D. & Mariotti, S. P. World Health Organization. Global data on visual |
| | impairments 2010. URL: http://www.who.int/blindness/GLOBALDATAFINALforweb.pdf |
| | [accessed 2013 Feb. 28][WebCite Cache] (2012). |
| 10 | Zrenner, E. Will retinal implants restore vision? Science 295, 1022-1025 (2002). |
| 11 | Martinez-Conde, S. & Macknik, S. L. Windows on the mind. Scientific American 297, |
| | 56-63 (2007). |
| 12 | Martinez-Conde, S. & Macknik, S. L. Shifting Focus. Scientific American Mind 22, 48-55 (2011). |
| 13 | Chen, Y. et al. Task difficulty modulates the activity of specific neuronal populations |
| | in primary visual cortex. Nat Neurosci 11, 974-982, doi: 10.1038/nn.2147 (2008). |
| 14 | Troncoso, X. G. et al. V1 neurons respond differently to object motion versus motion |
| | from eye movements. Nature communications 6, 8114, doi: 10.1038/ncomms9114 (2015). |
| 15 | Macknik, S. L. et al. Attention and awareness in stage magic: turning tricks into |
| | research. Nature Reviews Neuroscience 9, 871-879 (2008). |
| 16 | Martinez-Conde, S., Otero-Millan, J. & Macknik, S. L. The impact of microsaccades |
| | on vision: towards a unified theory of saccadic function. Nature Reviews Neuroscience |
| | 14, 83-96, doi: 10.1038/nrn3405 (2013). |
| 17 | Martinez-Conde, S. & Macknik, S. L. Opinion: Finding the plot in science storytelling |
| | in hopes of enhancing science communication. Proc Natl Acad Sci U S A 114, 8127- |
| | 8129, doi: 10.1073/pnas.1711790114 (2017). |
| 18 | Rieiro, H. et al. Optimizing the temporal dynamics of light to human perception. |
| | Proceedings of the National Academy of Sciences 109, 19828-19833 (2012). |
| 19 | Troncoso, X. G., Macknik, S. L., Otero-Millan, J. & Martinez-Conde, S. |
| | Microsaccades drive illusory motion in the Enigma illusion. Proceedings of the |
| | National Academy of Sciences 105, 16033-16038, doi: 0709389105 |
| | [pii]10.1073/pnas.0709389105 (2008). |
| 20 | Otero-Millan, J., Macknik, S. L., Langston, R. E. & Martinez-Conde, S. An |
| | oculomotor continuum from exploration to fixation. Proceedings of the National |
| | Academy of Sciences of the United States of America 110, 6175-6180, |
| | doi: 10.1073/pnas.1222715110 (2013). |
| 21 | Martinez-Conde, S., Macknik, S. L. & Heeger, D. J. An Enduring Dialogue between |
| | Computational and Empirical Vision. Trends Neurosci 41, 163-165, |
| | doi: 10.1016/j.tins.2018.02.005 (2018). |
| 22 | Martinez-Conde, S., Macknik, S. L., Troncoso, X. G. & Hubel, D. H. Microsaccades: a |
| | neurophysiological analysis. Trends Neurosci 32, 463-475 (2009). |
| 23 | McCamy, M. B. et al. Microsaccadic efficacy and contribution to foveal and peripheral |
| | vision. J Neurosci 32, 9194-9204, doi: 10.1523/JNEUROSCI.0515-12.2012 (2012). |
| 24 | Otero-Millan, J. et al. Distinctive features of saccadic intrusions and microsaccades in |
| | progressive supranuclear palsy. J Neurosci 31, 4379-4387, |
| | doi: 10.1523/JNEUROSCI.2600-10.2011 (2011). |
| 25 | Otero-Millan, J., Macknik, S. L. & Martinez-Conde, S. Microsaccades and blinks |
| | trigger illusory rotation in the “rotating snakes” illusion. J Neurosci 32, 6043-6051, |
| | doi: 10.1523/JNEUROSCI.5823-11.2012 (2012). |
| 26 | Otero-Millan, J., Macknik, S. L. & Martinez-Conde, S. Microsaccades and Blinks |
| | Trigger Illusory Rotation in the “Rotating Snakes” Illusion. The Journal of |
| | Neuroscience 32, 6043, doi: 10.1523/JNEUROSCI.5823-11.2012 (2012). |
| 27 | Otero-Millan, J., Troncoso, X. G., Macknik, S. L., Serrano-Pedraza, I. & Martinez- |
| | Conde, S. Saccades and microsaccades during visual fixation, exploration, and search: |
| | foundations for a common saccadic generator. J Vis 8, 21 21-18, |
| | doi: 10.1167/8.14.21/8/14/21/[pii] (2008). |
| 28 | Alexander, R. G., Waite, S., Macknik, S. L. & Martinez-Conde, S. What do |
| | radiologists look for? Advances and limitations of perceptual learning in radiologic |
| | search. Journal of Vision 20, 1-13, doi: 10.1167/jov.20.10.17 (2020). |
| 29 | Martinez-Conde, S. & Macknik, S. L. Fixational eye movements across vertebrates: |
| | comparative dynamics, physiology, and perception. Journal of Vision 8, 28-28 (2008). |
| 30 | Troncoso, X. G., Macknik, S. L. & Martinez-Conde, S. Microsaccades counteract |
| | perceptual filling-in. Journal of Vision 8, 15 11-19, doi: 10.1167/8.14.15 (2008). |
| 31 | Leal-Campanario, R. et al. Abnormal Capillary Vasodynamics Contribute to Ictal |
| | Neurodegeneration in Epilepsy. Scientific Reports 7, 43276, doi: 10.1038/srep43276 (2017). |
| 32 | Otero-Millan, J., Macknik, S. L., Serra, A., Leigh, R. J. & Martinez-Conde, S. |
| | Triggering mechanisms in microsaccade and saccade generation: a novel proposal. Ann |
| | N Y Acad Sci 1233, 107-116, doi: 10.1111/j.1749-6632.2011.06177.x (2011). |
| 33 | Di Stasi, L. L., Catena, A., Cañas, J. J., Macknik, S. L. & Martinez-Conde, S. Saccadic |
| | velocity as an arousal index in naturalistic tasks. Neuroscience & Biobehavioral |
| | Reviews 37, 968-975, doi: doi: 10.1016/j.neubiorev.2013.03.011 (2013). |
| 34 | Di Stasi, L. L. et al. Intersaccadic drift velocity is sensitive to short-term hypobaric |
| | hypoxia. Eur J Neurosci 39, 1384-1390 (2014). |
| 35 | Siegenthaler, E. et al. Task difficulty in mental arithmetic affects microsaccadic rates |
| | and magnitudes. Eur J Neurosci 39, 287-294, doi: 10.1111/ejn.12395 (2014). |
| 36 | Costela, F. M. et al. Changes in visibility as a function of spatial frequency and |
| | microsaccade occurrence. The European Journal of Neuroscience 45, 433-439 (2017). |
| 37 | Di Stasi, L. L. et al. Intersaccadic drift velocity is sensitive to short-term hypobaric |
| | hypoxia. The European Journal of Neuroscience 39, 1384-1390, |
| | doi: doi: 10.1111/ejn.12482 (2014). |
| 38 | Di Stasi, L. L. et al. Microsaccade and drift dynamics reflect mental fatigue. The |
| | European journal of neuroscience 38, 2389-2398, doi: 10.1111/ejn.12248 (2013). |
| 39 | Costela, F. M. et al. Characteristics of Spontaneous Square-Wave Jerks in the Healthy |
| | Macaque Monkey during Visual Fixation. PLoS One 10, e0126485, |
| | doi: 10.1371/journal.pone.0058535 (2013). |
| 40 | Costela, F. M. et al. Fixational eye movement correction of blink-induced gaze |
| | position errors. PloS one 9, e110889, doi: 10.1371/journal.pone.0110889 (2014). |
| 41 | Martinez-Conde, S., McCamy, M. B., Troncoso, X. G., Otero-Millan, J. & Macknik, S. |
| | L. Area V1 responses to illusory corner-folds in Vasarely's nested squares and the |
| | Alternating Brightness Star illusions. PLOS ONE 14, e0210941, |
| | doi: 10.1371/journal.pone.0210941 (2019). |
| 42 | McCamy, M. B. et al. Simultaneous recordings of human microsaccades and drifts |
| | with a contemporary video eye tracker and the search coil technique. PLoS One 10, |
| | e0128428, doi: 10.1371/journal.pone.0128428 (2015). |
| 43 | Cui, J., Otero-Millan, J., Macknik, S. L., King, M. & Martinez-Conde, S. Social |
| | misdirection fails to enhance a magic illusion. Front Hum Neurosci 5, 103, |
| | doi: 10.3389/fnhum.2011.00103 (2011). |
| 44 | Martinez-Conde, S. et al. Marvels of illusion: illusion and perception in the art of |
| | Salvador Dali. Front Hum Neurosci 9, 496, doi: 10.3389/fnhum.2015.00496 (2015). |
| 45 | Otero-Millan, J., Macknik, S. L., Robbins, A. & Martinez-Conde, S. Stronger |
| | misdirection in curved than in straight motion. Frontiers in human neuroscience 5, |
| | 133, doi: 10.3389/fnhum.2011.00133 (2011). |
| 46 | Troncoso, X. G., Macknik, S. L. & Martinez-Conde, S. Corner salience varies linearly |
| | with corner angle during flicker-augmented contrast: a general principle of corner |
| | perception based on Vasarely's artworks. Spatial Vision 22, 211-224 (2009). |
| 47 | Leigh, R. J. & Martinez-Conde, S. Tremor of the eyes, or of the head, in Parkinson's |
| | disease? Movement Disorders 28, 691-693 (2013). |
| 48 | Kapoula, Z. et al. Distinctive features of microsaccades in Alzheimer's disease and in |
| | mild cognitive impairment. Age (Dordrecht, Netherlands) 36, 535-543, |
| | doi: 10.1007/s11357-013-9582-3 (2014). |
| 49 | Costela, F. M., McCamy, M. B., Macknik, S. L., Otero-Millan, J. & Martinez-Conde, |
| | S. Microsaccades restore the visibility of minute foveal targets. PeerJ 1, e119, doi: doi: |
| | 10.7717/peerj.119 (2013). |
| 50 | McCamy, M. B. et al. Simultaneous recordings of ocular microtremor and |
| | microsaccades with a piezoelectric sensor and a video-oculography system. PeerJ 1, |
| | e14, doi: 10.7717/peerj.14 (2013). |
| 51 | McCamy, M. B., Najafian Jazi, A., Otero-Millan, J., Macknik, S. L. & Martinez- |
| | Conde, S. The effects of fixation target size and luminance on microsaccades and |
| | square-wave jerks. PeerJ 1, e9, doi: 10.7717/peerj.9 (2013). |
| 52 | Rieiro, H., Martinez-Conde, S. & Macknik, S. L. Perceptual elements in Penn & |
| | Teller's “Cups and Balls” magic trick. PeerJ 1, e19 (2013). |
| 53 | Shi, V., Cui, J., Troncoso, X. G., Macknik, S. L. & Martinez-Conde, S. Effect of |
| | stimulus width on simultaneous contrast. PeerJ 1, e146, doi: 10.7717/peerj.146 (2013). |
| 54 | Alexander, R. G., Macknik, S. L. & Martinez-Conde, S. Microsaccade Characteristics |
| | in Neurological and Ophthalmic Disease. Frontiers in neurology 9, 1-9, |
| | doi: 10.3389/fneur.2018.00144 (2018). |
| 55 | Otero-Millan, J., Optican, L. M., Macknik, S. L. & Martinez-Conde, S. Modeling the |
| | Triggering of Saccades, Microsaccades, and Saccadic Intrusions. Frontiers in |
| | neurology 9, 346, doi: 10.3389/fneur.2018.00346 (2018). |
| 56 | Waite, S. et al. A Review of Perceptual Expertise in Radiology-How it develops, how |
| | we can test it, and why humans still matter in the era of Artificial Intelligence. |
| | Academic Radiology 27, 26-38, doi: 10.1016/j.acra.2019.08.018 (2020). |
| 57 | Leal-Campanario, R., Martinez-Conde, S. & Macknik, S. L. In Vivo Fiber-Coupled |
| | Pre-Clinical Confocal Laser-scanning Endomicroscopy (pCLE) of Hippocampal |
| | Capillaries in Awake Mice. JOVE, e57220, doi: 10.3791/57220 (2020). |
| 58 | Ojemann, W. K. S. et al. A MRI-Based Toolbox for Neurosurgical Planning in |
| | Nonhuman Primates. JoVE, e61098, doi: doi: 10.3791/61098 (2020). |
| 59 | Di Stasi, L. L. et al. Saccadic eye movement metrics reflect surgical residents' fatigue. |
| | Annals of surgery 259, 824-829 (2014). |
| 60 | Di Stasi, L. L. et al. Effects of driving time on microsaccadic dynamics. Exp Brain Res |
| | 233, 599-605, doi: 10.1007/s00221-014-4139-y (2015). |
| 61 | Otero-Millan, J., Langston, R. E., Costela, F., Macknik, S. L. & Martinez-Conde, S. |
| | Microsaccade generation requires a foveal anchor. Journal of Eye Movement Research 12 (2019). |
| 62 | Martinez-Conde, S. & Macknik, S. L. Unchanging visions: the effects and limitations |
| | of ocular stillness. Philos Trans R Soc Lond B Biol Sci 372, |
| | doi: 10.1098/rstb.2016.0204 (2017). |
| 63 | Di Stasi, L. L. et al. Effects of long and short simulated flights on the saccadic eye |
| | movement velocity of aviators. Physiol Behav 153, 91-96, |
| | doi: 10.1016/j.physbeh.2015.10.024 (2016). |
| 64 | Di Stasi, L. L. et al. Task complexity modulates pilot electroencephalographic activity |
| | during real flights. Psychophysiology 52, 951-956, doi: 10.1111/psyp.12419 (2015). |
| 65 | Crist, R. E., Kapadia, M. K., Westheimer, G. & Gilbert, C. D. Perceptual-learning of |
| | spatial localization: Specificity for orientation, position, and context. Journal of |
| | Neurophysiology 78, 2889-2894 (1997). |
| 66 | Crist, R. E., Li, W. & Gilbert, C. D. Learning to see: experience and attention in |
| | primary visual cortex. Nature neuroscience 4, 519-525 (2001). |
| 67 | Gilbert, C., Ito, M., Kapadia, M. & Westheimer, G. Interactions between attention, |
| | context and learning in primary visual cortex. Vision Res 40, 1217-1226, doi: S0042- |
| | 6989(99)00234-5 [pii] (2000). |
| 68 | Gilbert, C. D., Sigman, M. & Crist, R. E. The neural basis of perceptual learning. |
| | Neuron 31, 681-697 (2001). |
| 69 | Ito, M., Westheimer, G. & Gilbert, C. D. Attention and perceptual learning modulate |
| | contextual influences on visual perception. Neuron 20, 1191-1197, doi: S0896- |
| | 6273(00)80499-7 [pii] (1998). |
| 70 | Li, W., Piech, V. & Gilbert, C. D. Perceptual learning and top-down influences in |
| | primary visual cortex. Nat Neurosci 7, 651-657, doi: 10.1038/nn1255 (2004). |
| 71 | Li, W., Piëch, V. & Gilbert, C. D. Learning to link visual contours. Neuron 57, 442- |
| | 451 (2008). |
| 72 | Sigman, M. & Gilbert, C. D. Learning to find a shape. Nat Neurosci 3, 264-269, |
| | doi: 10.1038/72979 (2000). |
| 73 | Sigman, M. et al. Top-down reorganization of activity in the visual pathway after |
| | learning a shape identification task. Neuron 46, 823-835, |
| | doi: 10.1016/j.neuron.2005.05.014 (2005). |
| 74 | Schoups, A. A., Vogels, R. & Orban, G. A. Human perceptual learning in identifying |
| | the oblique orientation: retinotopy, orientation specificity and monocularity. J Physiol |
| | 483 (Pt 3), 797-810 (1995). |
| 75 | Fahle, M. Perceptual learning: gain without pain? Nat Neurosci 5, 923-924, |
| | doi: 10.1038/nn1002-923 nn1002-923 [pii] (2002). |
| 76 | Fahle, M. & Poggio, T. Perceptual Learning. (The MIT Press, 2002). |
| 77 | Fahle, M. Perceptual learning: A case for early selection. Journal of vision 4, 4-4, |
| | doi: 10:1167/4.10.4/4/10/4/[pii] (2004). |
| 78 | Spang, K., Grimsen, C., Herzog, M. & Fahle, M. Orientation specificity of learning |
| | vernier discriminations. Vision research 50, 479-485 (2010). |
| 79 | Ghose, G. M., Yang, T. & Maunsell, J. H. Physiological correlates of perceptual |
| | learning in monkey V1 and V2. Journal of neurophysiology 87, 1867-1888 (2002). |
| 80 | Ju, N. et al. Spatiotemporal functional organization of excitatory synaptic inputs onto |
| | macaque V1 neurons. Nature communications 11, 697, doi: 10.1038/s41467-020-14501-y (2020). |
| 81 | Sadakane, O. et al. Long-Term Two-Photon Calcium Imaging of Neuronal Populations |
| | with Subcellular Resolution in Adult Non-human Primates. Cell Reports (2015). |
| 82 | Ruiz, O. et al. Optogenetics through windows on the brain in the nonhuman primate. |
| | Journal of neurophysiology 110, 1455-1467, doi: 10.1152/jn.00153.2013 (2013). |
| 83 | Mulliken, G. H. et al. Custom-fit radiolucent cranial implants for neurophysiological |
| | recording and stimulation. J Neurosci Methods 241, 146-154, |
| | doi: 10.1016/j.jneumeth.2014.12.011 (2015). |
| 84 | Seidemann, E. et al. Calcium imaging with genetically encoded indicators in behaving |
| | primates. Elife 5, doi: 10.7554/eLife.16178 (2016). |
| 85 | Yazdan-Shahmorad, A. et al. A Large-Scale Interface for Optogenetic Stimulation and |
| | Recording in Nonhuman Primates. Neuron 89, 927-939, doi: 10.1016/j.neuron.2016.01.013 (2016). |
| 86 | Li, M., Liu, F., Jiang, H., Lee, T. S. & Tang, S. Long-Term Two-Photon Imaging in |
| | Awake Macaque Monkey. Neuron 93, 1049-1057. e1043 (2017). |
| 87 | Yazdan-Shahmorad, A. et al. Widespread optogenetic expression in macaque cortex obtained |
| | with MR-guided, convection enhanced delivery (CED) of AAV vector to the thalamus. |
| | J Neurosci Methods 293, 347-358, doi: 10.1016/j.jneumeth.2017.10.009 (2018). |
| 88 | Nurminen, L., Merlin, S., Bijanzadeh, M., Federer, F. & Angelucci, A. Top-down |
| | feedback controls spatial summation and response amplitude in primate visual cortex. |
| | Nature communications 9, 2281, doi: 10.1038/s41467-018-04500-5 (2018). |
| 89 | Nassi, J. J., Lomber, S. G. & Born, R. T. Corticocortical feedback contributes to |
| | surround suppression in V1 of the alert primate. The Journal of neuroscience: the |
| | official journal of the Society for Neuroscience 33, 8504-8517, |
| | doi: 10.1523/JNEUROSCI.5124-12.2013 (2013). |
| 90 | AIM Photonics. AIM Silicon Photonics Process Design Kit (PDK), |
| | <http://www.aimphotonics.com/process-design-kit> (2020). |
| 91 | Ray, S. & Dasgupta, A. K. Light amplification by biofilm and its polarization |
| | dependence. bioRxiv (2020). |
| 92 | Kremkow, J. & Alonso, J.-M. Thalamocortical circuits and functional architecture. |
| | Annual review of vision science 4, 263-285 (2018). |
| 93 | Ju, N. et al. Spatiotemporal functional organization of excitatory synaptic inputs onto |
| | macaque V1 neurons. bioRxiv, 558163, doi: 10.1101/558163 (2019). |
| 94 | Lee, K. S., Huang, X. & Fitzpatrick, D. Topology of ON and OFF inputs in visual cortex enables |
| | an invariant columnar architecture. Nature 533, 90-94, doi: 10.1038/nature17941 (2016). |
| 95 | Hubel, D. H. & Wiesel, T. N. Uniformity of monkey striate cortex: a parallel relationship |
| | between field size, scatter, and magnification factor. J Comp Neurol 158, 295-305 (1974). |
| 96 | Joannopoulos, J. D., Johnson, S. G., Winn, J. N. & Meade, R. D. Molding the flow of |
| | light. Princeton Univ. Press, Princeton, NJ [ua] (2008). |
| 97 | Busch, K. & John, S. Liquid-crystal photonic-band-gap materials: the tunable |
| | electromagnetic vacuum. Phys Rev Lett 83, 967 (1999). |
| 98 | Lumerical. “High-Performance Nanophotonic Simulation Software.”, |
| | <https://www.lumerical.com/.> ( |
| 99 | Tabassum, N. et al. Engineered telecom emission and controlled positioning of Er3+ |
| | enabled by SiC nanophotonic structures. Nanophotonics 1 (2020). |
| 100 | Shim, E., Chen, Y., Masmanidis, S. & Li, M. Multisite silicon neural probes with |
| | integrated silicon nitride waveguides and gratings for optogenetic applications. |
| | Scientific Reports 6, 22693, doi: 10.1038/srep22693 (2016). |
| 101 | Gong, Q. & Hu, X. Photonic crystals: principles and applications. (CRC press, 2014). |
| 102 | Nambiar, S., Sethi, P. & Selvaraja, S. K. Grating-assisted fiber to chip coupling for |
| | SOI photonic circuits. Applied Sciences 8, 1142 (2018). |
| 103 | Marchetti, R., Lacava, C., Carroll, L., Gradkowski, K. & Minzioni, P. Coupling |
| | strategies for silicon photonics integrated chips. Photonics Research 7, 201-239 (2019). |
| 104 | Tabassum, N. et al. On-demand CMOS-compatible fabrication of ultrathin self-aligned |
| | SiC nanowire arrays. Nanomaterials 8, 906 (2018). |
| 105 | Wang, J., Xuan, Y., Weiner, A. M. & Qi, M. in 2014 Conference on Lasers and |
| | Electro-Optics (CLEO)-Laser Science to Photonic Applications. 1-2 (IEEE). |
| 106 | Sadakane, O. et al. Long-Term Two-Photon Calcium Imaging of Neuronal Populations |
| | with Subcellular Resolution in Adult Non-human Primates. Cell Rep 13, 1989-1999, |
| | doi: 10.1016/j.celrep.2015.10.050 (2015). |
| 107 | Jun, J. J. et al. Fully integrated silicon probes for high-density recording of neural |
| | activity. Nature 551, 232-236, doi: 10.1038/nature24636 (2017). |
| 108 | Rossant, C. et al. Spike sorting for large, dense electrode arrays. Nat Neurosci 19, 634- |
| | 641, doi: 10.1038/nn.4268 (2016). |
| 109 | Photonics, A. AIM Photonics, <http://www.aimphotonics.com/> ( |
| 110 | Institute, S. P. Centers + Programs, <https://sunypoly.edu/research/centers-programs.html> ( |
| 111 | Long, M., Jiang, W., Liu, D. & Yao, H. Contrast-dependent orientation discrimination |
| | in the mouse. Sci Rep 5, 15830, doi: 10.1038/srep15830 (2015). |
| 112 | Macknik, S. L. & Livingstone, M. S. Neuronal correlates of visibility and invisibility in |
| | the primate visual system. Nat Neurosci 1, 144-149, doi: 10.1038/393 (1998). |
| 113 | Martinez-Conde, S., Macknik, S. L. & Hubel, D. H. Microsaccadic eye movements and |
| | firing of single cells in the striate cortex of macaque monkeys [published erratum |
| | appears in Nat Neurosci 2000 Apr; 3(4): 409]. Nat Neurosci 3, 251-258 (2000). |
| 114 | Martinez-Conde, S., Macknik, S. L. & Hubel, D. H. The function of bursts of spikes |
| | during visual fixation in the awake primate lateral geniculate nucleus and primary |
| | visual cortex. Proc Natl Acad Sci U S A 99, 13920-13925, |
| | doi: 10.1073/pnas.212500599 212500599 [pii] (2002). |
| 115 | Macknik, S. L. & Martinez-Conde, S. The spatial and temporal effects of lateral |
| | inhibitory networks and their relevance to the visibility of spatiotemporal edges. |
| | Neurocomputing 58-60, 775-782, doi: 10.1016/j.neucom.2004.01.126 (2004). |
| 116 | Martinez-Conde, S., Macknik, S. L. & Hubel, D. H. The role of fixational eye |
| | movements in visual perception. Nature Reviews Neuroscience 5, 229-240 (2004). |
| 117 | Chen, Y. et al. Task difficulty modulates the activity of specific neuronal populations |
| | in primary visual cortex. Nat Neurosci, doi: 10.1038/nn.2147 (2006). |
| 118 | Leal-Campanario, R. et al. Abnormal Capillary Vasodynamics Contribute to Ictal |
| | Neurodegeneration in Epilepsy. Scientific Reports (2017/in-press). |
| 119 | Jazayeri, M., Lindbloom-Brown, Z. & Horwitz, G. D. Saccadic eye movements evoked |
| | by optogenetic activation of primate V1. Nature neuroscience 15, 1368-1370 (2012). |
| 120 | Nassi, J. J., Avery, M. C., Cetin, A. H., Roe, A. W. & Reynolds, J. H. Optogenetic |
| | activation of normalization in alert macaque visual cortex. Neuron 86, 1504-1517, |
| | doi: 10.1016/j.neuron.2015.05.040. (2015). |
| 121 | Burgess, S. A., Bouchard, M. B., Yuan, B. & Hillman, E. M. Simultaneous |
| | multiwavelength laminar optical tomography. Opt Lett 33, 2710-2712 (2008). |
| 122 | Hillman, E. M., Boas, D. A., Dale, A. M. & Dunn, A. K. Laminar optical tomography: |
| | demonstration of millimeter-scale depth-resolved imaging in turbid media. Opt Lett 29, |
| | 1650-1652 (2004). |
| 123 | Hillman, E. M. et al. Depth-resolved optical imaging and microscopy of vascular |
| | compartment dynamics during somatosensory stimulation. Neuroimage 35, 89-104, |
| | doi: 10.1016/j.neuroimage.2006.11.032 (2007). |
| 124 | Sirotin, Y. B., Hillman, E. M., Bordier, C. & Das, A. Spatiotemporal precision and |
| | hemodynamic mechanism of optical point spreads in alert primates. Proc Natl Acad Sci |
| | U S A 106, 18390-18395, doi: 0905509106 [pii] 10.1073/pnas.0905509106 (2009). |
| 125 | Klapoetke, N. C. et al. Independent optical excitation of distinct neural populations. |
| | Nature methods 11, 338 (2014). |
| 126 | Marcus, D., Herrick, R., Olsen, T., Horton, W. & Sheu, A. The XNAT Ecosystem. |
| | Front Neuroinform, doi: 10.3389/conf.fninf.2013.09.00102. |

A small sample of combinations set forth herein include the following: (A1) A method of restoring foveal vision, including: altering a first location of a neuron in a visual pathway of a patient in need thereof to form a light-emitting first location; and photostimulating the light-emitting first location to evoke neural responses which propagate along the neuron in the visual pathway, wherein the neural responses are formed with a light signal. (A2) The method of A1, wherein the light signal is emitted from a synthetic source such as a semiconductor device. (A3) The method of A1, wherein the first location includes neurons genetically encoded with one or more channelrhodopsin proteins to form photoreceptor cells within the first location. (A4) The method of A1, wherein the first location is downstream of the optic nerve such as in the brain or near the lateral geniculate nucleus (LGN) afferents in the foveal region of vision. (A5) The method of A1, wherein the first location is one or more individual LGN ON-vs. OFF-channel modules entering the primary visual area (V1) of the cerebral cortex. (B1) A method of treating a subject for ocular disorder, including: administering an effective amount of composition to a subject to alter one or more first locations of one or more neurons in a visual pathway to form a plurality of light-emitting first locations; and photostimulating the plurality of light-emitting first locations to evoke neural responses which propagate along the neuron in the visual pathway to improve or form vision. (B2) The method of B1, wherein the one or more first locations includes neurons genetically encoded with one or more channelrhodopsin proteins to form photoreceptor cells within the one or more first locations. (B3) The method of B1, wherein the one or more first locations are downstream of the optic nerve such as in the brain or near the lateral geniculate nucleus (LGN) afferents in the foveal region of vision. (B4) The method of B1, wherein the one or more first locations are at one or more individual LGN ON-vs. OFF-channel modules entering VI. (C1) A system comprising: a variable-intensity light source; an emitter assembly in communication with the variable intensity light source, the emitter assembly including: a switch matrix including: a plurality of waveguides in communication with the variable-intensity light source for receiving a light generated by the variable-intensity light source; and a plurality of optical switching devices positioned between and in communication with the plurality of waveguides, at least one of the plurality of optical switching devices receiving the light generated by the variable-intensity light source from one of the plurality of waveguides and providing the light to a distinct one of the plurality of waveguides based on a desired operation of the emitter assembly; a plurality of optical modulation devices in communication with the plurality of waveguides of the switch matrix, each of the plurality of optical modulation devices receiving and modulating the light generated by the variable-intensity light source; and a plurality of emitter devices in communication with a corresponding optical modulation device of the plurality of optical modulation devices, each of the plurality of emitter devices emitting the provided light generated by the variable-intensity light source toward a plurality of LGN-Channelrhodopsin neurons to stimulate light-emitting cortical neurons in communication with the plurality of LGN-Channelrhodopsin neurons; and a detector assembly positioned adjacent the emitter assembly, the detector assembly including: a plurality of semiconductor detector devices positioned adjacent each of the plurality of emitter devices of the emitter assembly and the plurality of stimulated light-emitting cortical neurons, each of the plurality of semiconductor detector devices detecting photons generated by the stimulated light-emitting cortical neurons; and a plurality of optical filtration devices disposed over each of the plurality of semiconductor detector devices, each of the plurality of optical filtration devices allowing a distinct, predetermined wavelength of the photons generated by the stimulated light-emitting cortical neurons to pass to the corresponding semiconductor detector device. (C2) The system of C1, wherein the plurality of optical switching devices include tunable micro-ring resonators (MRR). (C3) The system of C2, wherein the tunable MRR are operated using thermal-optics or electro-optics. (C4) The system of C1, wherein the plurality of optical modulation devices include tunable Mach-Zehnder interferometer (MZI), each of the plurality of tunable MZIs adjusting the intensity of the light generated by the variable-intensity light source before providing the light to a corresponding emitter device of the plurality of emitter devices. (C5) The system of C4, wherein the tunable MZI are operated using thermal-optics or electro-optics. (C6) The system of C1, wherein the emitter device includes at least one grating emitter. (C7) The system of C6, wherein the at least one grating emitter includes: a first grating emitter, and a second grating emitter disposed over the first grating emitter. (C8) The system of C1, wherein the plurality of optical filtration devices include a plurality of photonic crystals, each of the plurality of photonic crystals having a distinct periods of dielectric constants to allow the photon having the corresponding distinct, predetermined wavelength to pass to the corresponding semiconductor detector device. (C9) The system of C1, wherein the variable-intensity light source includes at least one laser diode that provides the light at a predetermined intensity, the predetermined intensity based on at least one of: a desired intensity of the light to be emitted by each of the plurality of emitter devices, or a number of emitter devices of the plurality of emitter devices that will emit the light at a single time. (C10) The system of C1, wherein only one of the plurality of emitter devices of the emitter assembly emits the light generated by the variable-intensity light source at a single time. (C11) The system of C1, wherein at least two of the plurality of emitter devices of the emitter assembly emit the light generated by the variable-intensity light source at a single time. (D1) A system comprising: an implant adapted for implantation in a user having a neocortex at least part of which has been made responsive to light, the neocortex including a plurality of columns forming an array of cortical columns capable of description by a cortical map characterizing, identifying or defining a location or topographical relationship and placement for respective ones of the plurality of columns; wherein the implant includes an emitter array; wherein the emitter array includes a plurality of emitters, wherein respective ones of the plurality of emitters are configured to emit light toward the array of cortical columns capable of description by the cortical map characterizing, identifying or defining a location or topographical relationship and placement for respective ones of the plurality of columns. (D2) The system of D1, wherein the system further comprises: the implant adapted for implantation in the user having a visual cortex that defines a component of the neocortex, the visual cortex including a plurality of hypercolumns forming an array of hypercolumns capable of description by the cortical map characterizing, identifying or defining the location or topographic relationship and placement for respective ones of the plurality of hypercolumns; wherein the emitter array includes a plurality of emitters, wherein respective ones of the plurality of emitters are configured to emit light toward the array of cortical hypercolumns capable of description by the cortical map characterizing, identifying or defining a location or topographical relationship and placement for respective ones of the plurality of hypercolumns. (D3) The system of D2, wherein the user is characterized by being vision impaired or blind user, and wherein the system is configured to present by the emitter array light emissions to stimulate hypercolumn quadrants of the array of hypercolumns, the light emissions based on frame image data obtained by a scene camera image sensor adapted to be worn by the user. (D4) The system of D2, wherein the user is characterized by being sighted user, and wherein the system is configured to present by the emitter array light emissions to stimulate hypercolumn quadrants of the array of hypercolumns, the light emissions based on frame image data transmitted to the user from a remote computing node. (D5) The system of D2, wherein a density of the plurality of emitters of the emitter array has greater density than the density of hypercolumn quadrants defining hypercolumns of the array of hypercolumns. (D6) The system of D2, wherein a density of the plurality of emitters of the emitter array is at least 2× greater than a density in a given dimension of the cortical hypercolumn quadrant map. (D7) The system of D2, wherein a density of the plurality of emitters of the emitter array is at least 4× greater than a density of the total hypercolumn quadrants of the array of hypercolumns. (D8) The system of D2, wherein the system runs a calibration process, wherein running of the calibration process includes discovering ones of the plurality of emitters that are aligned to a hypercolumn quadrant of the plurality of hypercolumns with minimized crosstalk between hypercolumn quadrants, and wherein as a result of the calibration process select ones of the plurality of the emitters that are determined to be not aligned to a hypercolumn quadrant of the plurality of hypercolumns are disabled. (D9) The system of D2, wherein the system is configured so that the implant emits using the emitter array a light field to the user in dependence on a received frame image data obtained using a camera image sensor. (D10) The system of D2, wherein the system is configured for presenting a frame of image data to the array of hypercolums with use of light emissions by the emitter array. (D11) The system of D2, wherein the system is configured for presenting a frame of image data to the array of hypercolums with use of light emissions by the emitter array, wherein the system is configured so that for performing the presenting the system controls first and second emitters which have been determined to be aligned to first and second hypercolumn quadrants of the array of hypercolumns. (D12) The system of D2, wherein the system is configured for presenting a frame of image data to the array of hypercolums with use of light emissions by the emitter array, wherein the system is configured so that for performing the presenting the system controls first and second emitters which have been determined to be aligned to first and second hypercolumn quadrants of the array of hypercolumns in dependence on an image data frame obtained using a scene camera image sensor. (D13) The system of D2, wherein the system is configured for presenting a frame of image data to the array of hypercolums with use of light emissions by the emitter array, wherein the system is configured so that for performing the presenting the system controls first and second emitters which have been determined to be aligned to first and second hypercolumn quadrants of the array of hypercolumns in dependence on one or more pixel value of an image data frame obtained using a scene camera image (D14) The system of D2, wherein the system includes an eye viewing camera image sensor having a field of view encompassing an eye of the user, wherein the system performs processing to determine a current eye position, and emits a scene representing light pattern using the emitter array to the array of hypercolumns in dependence on the current eye position. (D15) The system of D2, wherein the system includes a detector array, wherein the system runs a calibration process, wherein running of the calibration process includes discovering ones of the plurality of emitters that are aligned to a hypercolumn quadrants of the plurality of hypercolumns, wherein the discovering includes controlling first and second emitters of the emitter array to evoke perception of one or more of lightness, darkness, or gray at a certain cortical retinotopic position of the user's array of hypercolumns, and examining response signal information detected using one or more detector of the detector array, the examining including comparing the response signal information to targeted response data indicative of alignment of the first and second emitters with first and second hypercolumn quadrants of the array of hypercolumns. (E1) A system comprising: an implant adapted for implantation in a user having a neocortex at least part of which has been made responsive to light, the neocortex defined by a cortical map characterized by a plurality of columns; a plurality of emitters, wherein respective ones of the plurality of emitters are configured to emit light toward the cortical map characterized by the plurality of columns of the neocortex of the user; a plurality of detectors, wherein respective ones of the plurality of detectors are configured to detect response signals from brain tissue of the user that has been excited by a light emission of one or more emitter of the plurality of emitters. (E2) The system of E1, wherein the system comprises: the implant adapted for implantation in the user having a visual cortex of the neocortex, the visual cortex including a plurality of hypercolumns formed in an array of hypercolumns capable of description by the cortical map characterizing, identifying or defining the location or topographic relationship and placement for respective ones of the plurality of columns; wherein respective ones of the plurality of emitters are configured to emit light toward the array of hypercolumns; wherein respective ones of the plurality of detectors are configured to detect response signals from brain tissue of the user that has been excited by a light emission of one or more emitter of the plurality of emitters. (E3) The system of E1 or E2, wherein the plurality of emitters and the plurality of detectors are co-located in the implant adapted for implantation in the user. (E4) The system of E1 or E2, wherein the implant adapted for implantation in the user includes a housing, and wherein the plurality of emitters and the plurality of detectors are disposed in the housing. (E5) The system of E1 or E2, wherein the system is configured to read out a frame of image data from the plurality of detectors based on response signals detected by detectors of the plurality of detectors, and wherein the system is configured to transmit the frame of image data to a computing node remote from the user. (E6) The system of E1 or E2, wherein the system is configured to read out a moving frame of image data from the plurality of detectors based on response signals detected by detectors of the plurality of detectors, and wherein the system is configured to transmit the moving frame of image data to a computing node remote from the user. (E7) The system of E1 or E2, wherein the system is configured so that power delivery by a respective emitter of the plurality of emitters is regulated in dependence on a response signal detected by one or more detector of the plurality of detectors in response to excitation of brain tissue by the respective emitter. (E8) The system of E1 or E2, wherein the system is configured so that power delivery by a respective emitter of the plurality of emitters is regulated in dependence on a response signal detected by one or more detector of the plurality of detectors in response to excitation of brain tissue by the respective emitter, and wherein the power delivery by the respective emitter is controlled using one or more of emission amplitude control or emission pulse width modulation. (E9) The system of E1 or E2, wherein the system is configured so that power delivery by a respective emitter of the plurality of emitters is regulated in dependence on a response signal detected by one or more detector of the plurality of detectors in response to excitation of brain tissue by the respective emitter, and wherein the power delivery by the respective emitter is controlled using emission pulse width modulation. (E10) The system of E1 or E2, wherein the system is configured so that power delivery by respective emitters of the plurality of emitters is regulated in dependence on a response signal detected by one or more detector of the plurality of detectors in response to excitation of brain tissue by respective emitters, and wherein the power delivery by the respective emitters is established so that different emitters of the plurality of emitters are controlled to have different associated power delivery levels. (E11) The system of E1 or E2, wherein the system includes a plurality of optical modulation devices for producing emissions by the plurality of emitters. (E12) The system of E1 or E2, wherein the system is configured so that power delivery by respective emitters of the plurality of emitters is regulated iteratively over time in dependence on a response signal iteratively detected by one or more detector of the plurality of detectors in response to iterative excitation of brain tissue by the respective emitters, and wherein the power delivery by the respective emitters is iteratively established over time so that for respective artificial frame presentment periods, different emitters of the plurality of emitters are controlled to have different associated power delivery levels, and further so that, for respective ones of the frame presentment periods, power delivery levels associated to the different emitters change. (E13) The system of E1 or E2, wherein the system includes, for producing emissions by the plurality of emitters, optical switching devices receiving light generated by the variable-intensity light source and providing the light to a distinct one of a plurality of waveguides. (E14) The system of E2, wherein the system is configured to perform a calibration process in which an emission by an emitter of the plurality of emitters is controlled, and a response signal detected by a detector of the plurality of detectors is examined to determine whether the emitter is aligned to a hypercolumn quadrant of the array of hypercolumns. (E15) The system of E2, wherein the system is configured to perform a calibration process in which emitters not aligned to hypercolumn quadrants of the array of hypercolumns are discovered and disabled. (E16) The system of E2, wherein the system is configured to perform a calibration process in which emitters of the plurality of emitters not aligned to hypercolumn quadrants of the array of hypercolumns are discovered and disabled, and wherein the system is further configured to perform an artificial viewing session, wherein for performance of the artificial viewing session, emitters of the plurality of emitters that have not been disabled by the calibration process are selectively controlled to present one or more frame of image data to the array of hypercolumns. (E17) The system of E2, wherein the system is configured to perform a calibration process in which an emission by an emitter of the plurality of emitters is controlled, and a response signal detected by a detector of the plurality of detectors is examined to determine whether the emitter is aligned to a hypercolumn quadrant of the array of hypercolumns, and wherein the system is configured so that in response to a determination that the emitter is not aligned to the hypercolumn quadrant, disabling the emitter. (E18) The system of E1 or E2, wherein the system is configured to identify a source location of a response signal based on a determined color of the response signal, wherein the response signal is detected with use of the detector of the plurality of detectors. (E19) The system of E1 or E2, wherein the system includes plurality of optical modulation devices receiving and modulating light generated by a variable-intensity light source; wherein respective ones of the plurality of emitters is in communication with a corresponding optical modulation device of the plurality of optical modulation devices. (E20) The system of E1 or E2, wherein respective ones of the plurality of detectors are placed adjacent to a detector of the plurality of detectors, and a plurality of optical filtration devices, wherein respective ones of the plurality of optical filtration devices are disposed over respective ones of the plurality of detectors, wherein respective ones of the plurality of optical filtration devices are tunable to allow a distinct, predetermined wavelength to pass through to its corresponding detector of the plurality of detectors.

Embodiments of the present disclosure drive stimulation in the primary visual cortex (V1) by activating thalamic (lateral geniculate nucleus; LGN) neuronal afferents entering V1 with synaptic precision, as in natural vision. Accordingly, the present disclosure relates to formulations, methods and devices for the restoration of visual responses, reducing or preventing the development or the risk of ocular disorders, and/or alleviating or curing ocular disorders including blindness in a subject such as a human or other non-human mammal or other animal.

This written description uses examples to disclose the subject matter, and also to enable any person skilled in the art to practice the subject matter, including making and using any devices or systems and performing any incorporated methods. The patentable scope of the subject matter is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

It is to be understood that the above description is intended to be illustrative, and not restrictive. For example, the above-described examples (and/or aspects thereof) may be used in combination with each other. In addition, many modifications may be made to adapt a particular situation or material to the teachings of the various examples without departing from their scope. While the dimensions and types of materials described herein are intended to define the parameters of the various examples, they are by no means limiting and are merely exemplary. Many other examples will be apparent to those of skill in the art upon reviewing the above description. The scope of the various examples should, therefore, be determined with reference to the appended claims, along with the full scope of equivalents to which such claims are entitled. In the appended claims, the terms “including” and “in which” are used as the plain-English equivalents of the respective terms “comprising” and “wherein.” Moreover, in the following claims, the terms “first,” “second,” and “third,” etc. are used merely as labels, and are not intended to impose numerical requirements on their objects. Forms of term “based on” herein encompass relationships where an element is partially based on as well as relationships where an element is entirely based on. Forms of the term “defined” encompass relationships where an element is partially defined as well as relationships where an element is entirely defined. Further, the limitations of the following claims are not written in means-plus-function format and are not intended to be interpreted based on the 35 U.S.C. § 112(f), unless and until such claim limitations expressly use the phrase “means for” followed by a statement of function void of further structure. It is to be understood that not necessarily all such objects or advantages described above may be achieved in accordance with any particular example. Thus, for example, those skilled in the art will recognize that the systems and techniques described herein may be embodied or carried out in a manner that achieves or optimizes one advantage or group of advantages as taught herein without necessarily achieving other objects or advantages as may be taught or suggested herein.

While the subject matter has been described in detail in connection with only a limited number of examples, it should be readily understood that the subject matter is not limited to such disclosed examples. Rather, the subject matter can be modified to incorporate any number of variations, alterations, substitutions or equivalent arrangements not heretofore described, but which are commensurate with the spirit and scope of the subject matter. Additionally, while various examples of the subject matter have been described, it is to be understood that aspects of the disclosure may include only some of the described examples. Also, while some examples are described as having a certain number of elements it will be understood that the subject matter can be practiced with less than or greater than the certain number of elements. Accordingly, the subject matter is not to be seen as limited by the foregoing description but is only limited by the scope of the appended claims.

1. A system comprising:
an implant adapted for implantation in a user having a neocortex at least part of which has been made responsive to light, the neocortex including a plurality of columns forming an array of cortical columns capable of description by a cortical map characterizing, identifying or defining a location or topographical relationship and placement for respective ones of the plurality of columns;
wherein the implant includes an emitter array;
wherein the emitter array includes a plurality of emitters, wherein respective ones of the plurality of emitters are configured to emit light toward the array of cortical columns capable of description by the cortical map characterizing, identifying or defining a location or topographical relationship and placement for respective ones of the plurality of columns.

2. The system of claim 1, wherein the system further comprises:
the implant adapted for implantation in the user having a visual cortex that defines a component of the neocortex, the visual cortex including a plurality of hypercolumns forming an array of hypercolumns capable of description by the cortical map characterizing, identifying or defining the location or topographic relationship and placement for respective ones of the plurality of hypercolumns;
wherein the emitter array includes a plurality of emitters, wherein respective ones of the plurality of emitters are configured to emit light toward the array of cortical hypercolumns capable of description by the cortical map characterizing, identifying or defining a location or topographical relationship and placement for respective ones of the plurality of hypercolumns.

3. The system of claim 2, wherein the user is characterized by being vision impaired or blind user, and wherein the system is configured to present by the emitter array light emissions to stimulate hypercolumn quadrants of the array of hypercolumns, the light emissions based on frame image data obtained by a scene camera image sensor adapted to be worn by the user.

4. The system of claim 2, wherein the user is characterized by being sighted user, and wherein the system is configured to present by the emitter array light emissions to stimulate hypercolumn quadrants of the array of hypercolumns, the light emissions based on frame image data transmitted to the user from a remote computing node.

5. The system of claim 2, wherein a density of the plurality of emitters of the emitter array has greater density than the density of hypercolumn quadrants defining hypercolumns of the array of hypercolumns.

6. The system of claim 2, wherein the system is further characterized by one or more of the following selected from the group consisting of (a) a density of the plurality of emitters of the emitter array is at least 2× greater than a density in a given dimension of the cortical hypercolumn quadrant map, and (b) a density of the plurality of emitters of the emitter array is at least 4× greater than a density of the total hypercolumn quadrants of the array of hypercolumns.

7. (canceled)

8. The system of claim 2, wherein the system runs a calibration process, wherein running of the calibration process includes discovering ones of the plurality of emitters that are aligned to a hypercolumn quadrant of the plurality of hypercolumns with minimized crosstalk between hypercolumn quadrants, and wherein as a result of the calibration process select ones of the plurality of the emitters that are determined to be not aligned to a hypercolumn quadrant of the plurality of hypercolumns are disabled.

9. The system of claim 2, wherein the system is further characterized by one or more of the following selected from the group consisting of (a) the system is configured so that the implant emits using the emitter array a light field to the user in dependence on a received frame image data obtained using a camera image sensor, (b) the system is configured for presenting a frame of image data to the array of hypercolums with use of light emissions by the emitter array, and (c) the system is configured for presenting a frame of image data to the array of hypercolums with use of light emissions by the emitter array, wherein the system is configured so that for performing the presenting the system controls first and second emitters which have been determined to be aligned to first and second hypercolumn quadrants of the array of hypercolumns.

10. (canceled)

11. (canceled)

12. The system of claim 2, wherein the system is further characterized by one or more of the following selected from the group consisting of (a) the system is configured for presenting a frame of image data to the array of hypercolums with use of light emissions by the emitter array, wherein the system is configured so that for performing the presenting the system controls first and second emitters which have been determined to be aligned to first and second hypercolumn quadrants of the array of hypercolumns in dependence on an image data frame obtained using a scene camera image sensor, and (b) the system is configured for presenting a frame of image data to the array of hypercolums with use of light emissions by the emitter array, wherein the system is configured so that for performing the presenting the system controls first and second emitters which have been determined to be aligned to first and second hypercolumn quadrants of the array of hypercolumns in dependence on one or more pixel value of an image data frame obtained using a scene camera image.

13. (canceled)

14. The system of claim 2, wherein the system includes an eye viewing camera image sensor having a field of view encompassing an eye of the user, wherein the system performs processing to determine a current eye position, and emits a scene representing light pattern using the emitter array to the array of hypercolumns in dependence on the current eye position.

15. The system of claim 2, wherein the system includes a detector array, wherein the system runs a calibration process, wherein running of the calibration process includes discovering ones of the plurality of emitters that are aligned to a hypercolumn quadrants of the plurality of hypercolumns, wherein the discovering includes controlling first and second emitters of the emitter array to evoke perception of one or more of lightness, darkness, or gray at a certain cortical retinotopic position of the user's array of hypercolumns, and examining response signal information detected using one or more detector of the detector array, the examining including comparing the response signal information to targeted response data indicative of alignment of the first and second emitters with first and second hypercolumn quadrants of the array of hypercolumns.

16. A system comprising:
an implant adapted for implantation in a user having a neocortex at least part of which has been made responsive to light, the neocortex defined by a cortical map characterized by a plurality of columns;
a plurality of emitters, wherein respective ones of the plurality of emitters are configured to emit light toward the cortical map characterized by the plurality of columns of the neocortex of the user;
a plurality of detectors, wherein respective ones of the plurality of detectors are configured to detect response signals from brain tissue of the user that has been excited by a light emission of one or more emitter of the plurality of emitters.

17. The system of claim 16, wherein the system comprises:
the implant adapted for implantation in the user having a visual cortex of the neocortex, the visual cortex including a plurality of hypercolumns formed in an array of hypercolumns capable of description by the cortical map characterizing, identifying or defining the location or topographic relationship and placement for respective ones of the plurality of columns;
wherein respective ones of the plurality of emitters are configured to emit light toward the array of hypercolumns;
wherein respective ones of the plurality of detectors are configured to detect response signals from brain tissue of the user that has been excited by a light emission of one or more emitter of the plurality of emitters.

18. The system of claim 16, wherein the system is further characterized by one or more of the following selected from the group consisting of (a) the plurality of emitters and the plurality of detectors are co-located in the implant adapted for implantation in the user, (b) the implant adapted for implantation in the user includes a housing, and wherein the plurality of emitters and the plurality of detectors are disposed in the housing, (c) the system is configured to read out a frame of image data from the plurality of detectors based on response signals detected by detectors of the plurality of detectors, and wherein the system is configured to transmit the frame of image data to a computing node remote from the user, and (d) the system is configured to read out a moving frame of image data from the plurality of detectors based on response signals detected by detectors of the plurality of detectors, and wherein the system is configured to transmit the moving frame of image data to a computing node remote from the user.

19. (canceled)

20. (canceled)

21. (canceled)

22. The system of claim 16, wherein the system is configured so that power delivery by a respective emitter of the plurality of emitters is regulated in dependence on a response signal detected by one or more detector of the plurality of detectors in response to excitation of brain tissue by the respective emitter.

23. The system of claim 16, wherein the system is further characterized by on one or more of the following selected from the group consisting of (a) the system is configured so that power delivery by a respective emitter of the plurality of emitters is regulated in dependence on a response signal detected by one or more detector of the plurality of detectors in response to excitation of brain tissue by the respective emitter, and wherein the power delivery by the respective emitter is controlled using one or more of emission amplitude control or emission pulse width modulation(b) the system is configured so that power delivery by a respective emitter of the plurality of emitters is regulated in dependence on a response signal detected by one or more detector of the plurality of detectors in response to excitation of brain tissue by the respective emitter, and wherein the power delivery by the respective emitter is controlled using emission pulse width modulation, (c) the system is configured so that power delivery by respective emitters of the plurality of emitters is regulated in dependence on a response signal detected by one or more detector of the plurality of detectors in response to excitation of brain tissue by respective emitters, and wherein the power delivery by the respective emitters is established so that different emitters of the plurality of emitters are controlled to have different associated power delivery levels, (d) the system includes a plurality of optical modulation devices for producing emissions by the plurality of emitters, and (e) the system is configured so that power delivery by respective emitters of the plurality of emitters is regulated iteratively over time in dependence on a response signal iteratively detected by one or more detector of the plurality of detectors in response to iterative excitation of brain tissue by the respective emitters, and wherein the power delivery by the respective emitters is iteratively established over time so that for respective artificial frame presentment periods, different emitters of the plurality of emitters are controlled to have different associated power delivery levels, and further so that, for respective ones of the frame presentment periods, power delivery levels associated to the different emitters change.

24. (canceled)

25. (canceled)

26. (canceled)

27. (canceled)

28. The system of claim 16, wherein the system is further characterized by one or more of the following selected from the group consisting of (a) the system includes, for producing emissions by the plurality of emitters, optical switching devices receiving light generated by the variable-intensity light source and providing the light to a distinct one of a plurality of waveguides, (b) the system is configured to perform a calibration process in which an emission by an emitter of the plurality of emitters is controlled, and a response signal detected by a detector of the plurality of detectors is examined to determine whether the emitter is aligned to a hypercolumn quadrant of the array of hypercolumns, (c) the system is configured to perform a calibration process in which emitters not aligned to hypercolumn quadrants of the array of hypercolumns are discovered and disabled, (d) the system is configured to perform a calibration process in which emitters of the plurality of emitters not aligned to hypercolumn quadrants of the array of hypercolumns are discovered and disabled, and wherein the system is further configured to perform an artificial viewing session, wherein for performance of the artificial viewing session, emitters of the plurality of emitters that have not been disabled by the calibration process are selectively controlled to present one or more frame of image data to the array of hypercolumns, and (e) the system is configured to perform a calibration process in which an emission by an emitter of the plurality of emitters is controlled, and a response signal detected by a detector of the plurality of detectors is examined to determine whether the emitter is aligned to a hypercolumn quadrant of the array of hypercolumns, and wherein the system is configured so that in response to a determination that the emitter is not aligned to the hypercolumn quadrant, disabling the emitter.

29. (canceled)

30. (canceled)

31. (canceled)

32. The system of claim 16, wherein the system is configured to identify a source location of a response signal based on a determined color of the response signal, wherein the response signal is detected with use of the detector of the plurality of detectors.

35. The system of claim 16 wherein respective ones of the plurality of detectors are placed adjacent to a detector of the plurality of detectors, and a plurality of optical filtration devices, wherein respective ones of the plurality of optical filtration devices are disposed over respective ones of the plurality of detectors, wherein respective ones of the plurality of optical filtration devices are tunable to allow a distinct, predetermined wavelength to pass through to its corresponding detector of the plurality of detectors.

36. The system of claim 16, wherein the system includes plurality of optical modulation devices receiving and modulating light generated by a variable-intensity light source; wherein respective ones of the plurality of emitters is in communication with a corresponding optical modulation device of the plurality of optical modulation devices.
